(19) United States
(12) Patent Application Publication
(10) Pub. No.: US 2025/0084406 A1
(43) Pub. Date: Mar. 13, 2025

(54) REVERSIBLE HETERODIMERIZING SYSTEMS AS EFFECTORS FOR FEEDBACK CONTROL

(71) Applicant: The Regents of the University of California, Oakland, CA (US)

(72) Inventors: Taylor Hanh NGUYEN, San Francisco, CA (US); Galen DODS, San Francisco, CA (US); Hana EL-SAMAD, San Francisco, CA (US); Andrew H. NG, San Francisco, CA (US)

(21) Appl. No.: 18/728,069

(22) PCT Filed: Jan. 17, 2023
(86) PCT No.: PCT/US2023/060790

§ 371 Date: Jul. 10, 2024

Related U.S. Application Data
(60) Provisional application No. 63/301,422, filed on Jan. 20, 2022.

(51) Int. Cl. C12N 15/10; G01N 33/68

(52) U.S. Cl. CPC C12N 15/1086; C12N 15/1055; G01N 33/6845

(57) ABSTRACT

Provided herein is a molecular switch and a variety of feedback and feed-forward circuits that employ a split protein (e.g., a split transcription factor) and monomers from designed heterodimers In some embodiments, the cell may contain a first polypeptide comprising a first part of a split protein and a monomer of a designed heterodimer; (b) a second polypeptide comprising a second part of a split protein and a monomer of a designed heterodimer; and (c) a third polypeptide comprising a monomer of a designed heterodimer, not containing the first or second parts of the split protein. In these embodiments, (a) and (b) bind to each other, and (c) binds to (a) or (b), thereby inactivating the reconstituted split protein. Expression of (c) is regulated by the activity of the reconstituted split protein. Various circuits are also provided.

### CROSS-REFERENCING

This application claims the benefit of U.S. provisional application Ser. No. 63/301,422, filed on Jan. 20, 2022, which application is incorporated by reference herein in its entirety.

### GOVERNMENT RIGHTS

This invention was made with government support under grant HR0011-16-2-0045 awarded by The Defense Advanced Research Projects Agency. The government has certain rights in the invention.

### INCORPORATION BY REFERENCE OF SEQUENCE LISTING PROVIDED AS AN XML FILE

A Sequence Listing is provided herewith as a Sequence Listing XML, “UCSF-668WO_SEQ_LIST” created on Jan. 12, 2023 and having a size of 384 kilobytes. The contents of the Sequence Listing XML are incorporated by reference herein in their entirety.

### BACKGROUND

Protein-protein interactions play a critical role in regulating signal transduction.¹ Many transcription factors utilize dimerization to control their activity, such as basic helix-loop-helix (bHLH) proteins, which play important roles in differentiation and whose dysregulation can cause cancer.² One such bHLH, MyoD, can either homodimerize or heterodimerize with other bHLH's, including E47 and E12, to activate transcription. Other bHLHs, including Group D bHLHs, contain the HLH domain needed for dimerization but lack the basic region needed for transcriptional activation. Consequently, Group D proteins can bind other bHLHs to negatively regulate their activity. For example, the Group D Id protein is able to preferentially bind MyoD, E47, or E12 to attenuate their ability to bind DNA, thus repressing their transcriptional programs.^{3,4} The relative abundances of these proteins determines the differentiation state of muscle cells. This example showcases how both transcriptional activation and repression can be highly regulated through the joining and competition of transcription factor dimerization. Such protein-protein interactions are therefore promising engineering targets for achieving complex and novel cellular functions.

Although protein-based circuits are ubiquitously used for endogenous biological regulation, methods for synthetic modulation of protein-protein interactions remain limited. Previous work in synthetic biology has focused on using high-affinity binders to create activation signals. This highly modular strategy can be used to reconstitute split proteins or colocalize signaling molecules. This functionality has enabled the control of transcription, localization, and proteolysis;⁵⁻¹⁰ the tuning of ultrasensitivity and cooperativity;¹¹⁻¹⁵ the rewiring of endogenous signalling pathways;¹⁶⁻¹⁹ the optimization of metabolic pathways;²⁰⁻²² and the modulation of CAR activity and downstream T cell responses.^{23,24} Many of these applications utilize SYNZIPs, which are a library of highly-characterized synthetic bZIP proteins.²⁵ However, far less work has been dedicated to building inhibitory binding reactions that can interrupt the function of the dimerized partners, like those seen in endogenous pathways. Furthermore, inhibitory binding could be used in synthetic circuits to tune dose responses or implement composable negative regulation and feedback, enabling the construction of complex circuits that more closely approximate endogenous signaling.

Recently, a library of de novo Designed HeteroDimers (DHDs) was computationally designed using Rosetta HBNet.²⁶ Each heterodimer pair shares the same four-helix bundle backbone structure, and specificity was achieved by incorporating asymmetric hydrogen-bond networks into the backbone. Monomers were generated by using short loops to connect pairs of helices, and six heterodimer pairs were observed to be fully orthogonal via a yeast two-hybrid assay. Each designed pair is referred to by a number, and each monomer within a pair is designated as A or B, i.e., 37A and 37B make up the designed 37 on-target interaction. The functionality of the DHD library was demonstrated via the construction of multi-component protein logic gates that regulate split-protein activity and transcription in yeast and T-cells.²⁴

The functionality of DHDs is extended to provide a variety of molecular switches and circuits.

### SUMMARY

Cells that comprise a variety of molecular switches, feedback circuits and feedforward circuit circuits are described herein. In some embodiments, a cell may comprise: (a) a first polypeptide comprising a first part of a split protein and a first monomer of a designed heterodimer; (b) a second polypeptide comprising a second part of a split protein and a second monomer of a designed heterodimer; and (c) a third polypeptide comprising a third monomer of a designed heterodimer. In these embodiments, (a) and (b) bind to each other via a relatively low affinity interaction between their monomers to produce a reconstituted split protein that has an activity is not provided by either (a) or (b) alone; and (c) binds to (a) and/or (b) via a relatively high affinity between their monomers, thereby inactivating the reconstituted split protein.

In other embodiments, the cell may comprise a feedback circuit. In these embodiments, the cell may comprise: (a) a first polypeptide comprising a first part of a split protein and a monomer of a designed heterodimer; (b) a second polypeptide comprising a second part of a split protein and a monomer of a designed heterodimer; and (c) a third polypeptide comprising a monomer of a designed heterodimer, not containing the first or second parts of the split protein. In these embodiments, (a) and (b) bind to each other via an interaction between their monomers to produce a reconstituted split protein that has an activity is not provided by either (a) or (b) alone; (c) binds to (a) or (b) via an interaction between their monomers, thereby inactivating the reconstituted split protein; and expression of (c) is regulated by the activity of the reconstituted split protein.

In other embodiments, the cell may comprise a feedforward circuit. In these embodiments, the cell may comprise: (a) a first polypeptide comprising a first part of a split protein and a monomer of a designed heterodimer; (b) a second polypeptide comprising a second part of a split protein and a monomer of a designed heterodimer; (c) a third polypeptide comprising a monomer of a designed heterodimer, not linked to the first or second parts of the split protein, and (d) an actuating protein that, in response to an external stimulus, independently activates expression of (c) and at least one of (a) and (b). In these embodiments, (a) and (b) bind to each other via an interaction between their monomers to produce a reconstituted split protein that has an activity is not provided by either (a) or (b) alone; and (c) binds to (a) or (b) via an interaction between their monomers, thereby inactivating the reconstituted split protein.

### BRIEF DESCRIPTION OF THE DRAWINGS

FIGS. 1A-1D: A split transcription factor system enables the quantification of DHD interactions via fluorescence.

FIG. 1A: Cartoon of the split TF system. Different DHDs are fused to the ZF43_8 and VP16 species, and these are induced by the addition of E2 and Pg, respectively. Interacting DHDs reconstitute the transcription factor complex and induce YFP (Venus) expression from the cognate promoter (p43_8).

FIG. 1B: YFP fluorescence generated by the reconstitution of the split TF using different termini fusions of the 37B: 37A pair. The label of “N:N” denotes the orientation where both DHDs were fused to the N-termini of VP16 (AD) and ZF43_8 (DHD), whereas “N:C” denotes the orientation where DHD is fused the N-terminus of VP16 (AD) and the C-terminus of ZF43_8 (DBD). Constructs were induced with a saturating dose of 36 nM E2 and 256 nM Pg. Steady-state YFP measurements at 6 hours post-induction are reported. Values represent the mean and s.d. of 3 biological replicates.

FIG. 1C: YFP fluorescence dose response as a function of Pg for the 37 (left) and 154 (right) DHD pairs at select values of E2. Both cases show a decrease in expression with increasing amounts of the DBD species (E2 amount). Values represent the mean and s.d. of 3 biological replicates and data were collected 6 hours after E2 and Pg induction.

FIG. 1D: All-by-all interaction matrix for 4 DHD pairs. Designed interactions are outlined in red. Values represent the mean YFP fold-change between when both species are induced (72 nM E2 and 128 nM Pg) and neither species is induced (OnM E2 and Pg). Data were collected 6 hours after E2 and Pg induction. Values represent the mean of 3 biological replicates.

FIGS. 2A-2F: Characterization of competitive displacement via DHD dominant negatives (DN).

FIGS. 2A-2B: Cartoons of the system used to test the dominant negative. Estradiol induces the production of the off-target interacting DHDs that reconstitute the transcription factor. Progesterone induces the production of DN that can outcompete the bound DHDs. The DN interferes with complex formation or integrity by binding to either the AD (A) or DBD (B) species, and therefore turns off YFP-cODC degron expression observed through a decrease in YFP fluorescence.

FIG. 2C: Left, timeline of inductions. Both E2 and Pg are given at time 0 and YFP expression was measured 6 hours after induction.

FIGS. 2D-2E: Steady-state responses of each circuit layout in A-B, respectively. Each strain is given a saturating amount of E2 (36 nM) and a range of Pg concentrations. (FIG. 2F: Left, timeline of inductions. E2 is given at time 0 allowing the DHD complex to form, while Pg is given 4 hours laters to test whether the DN can interfere with the complex. YFP expression was measured 8 hours after induction with E2 (4 hours after induction with Pg). Right, YFP fluorescence as a function of time for each layout in panels A-B (37B: 154B in blue, DN is 154A and 154B: 155A in red, DN is 154A). The data show DN can displace pre-formed complexes as evidenced by a decrease in YFP fluorescence. Measurements were taken every 30 minutes for 4 hours beginning immediately after Pg induction. YFP expression was normalized by the expression at time 0. For all panels, values represent the mean and s.d. of 3 biological replicates. The lines connecting points were added to aid visualization.

FIGS. 3A-3B: Design and implementation of a negative feedback circuit using DN competitive displacement.

FIG. 3A: Schematic of the synthetic feedback circuit implemented with the competitive displacement strategy. E2 acts as the input and induces expression of the DHD transcription factor complex. The transcription factor then induces the production of Z3PM and RFP (an internal readout of transcription factor activity). Z3PM in turn induces expression of the 2×YFP output (YFP-cODC increases the turnover rate of YFP to degrade old YFP molecules and approximate current YFP output). The Feedback circuit produces the DN (DN-cODC degron) from the pZ3 promoter. The Feedback circuit is compared to two No Feedback circuits where the DN-cODC is produced from a constitutive promoter (pC). pC can either be pRPL18B (noted as pRPL18B-DN) or a pRNR2 (noted as pRNR2-DN) in subsequent panels.

FIG. 3B: RFP and YFP values after 8 hours of circuit induction for an input E2 of 144 nM, plotted as a function of Pg. Shown are the feedback (red and blue) and no feedback data (light grey for pRPL18B-DN and dark grey for pRNR2-DN)

FIG. 3C: The output of the Feedback circuit can be tuned with different E2 inputs. Feedback (red and blue) and both No Feedback (grays) circuits were induced with a full range of E2 and Pg for 8 hours. Fluorescence values were normalized to the maximum fluorescence value of each channel of each circuit, to allow for comparisons between circuits with different dynamic ranges. Lines were added to aid visualization. All values represent the mean and s.d. of 3 biological replicates.

FIGS. 4A and 4B: A competition mathematical model recapitulates the paradoxical effect of higher DBD part expression resulting in lower transcriptional output. Using the synthesis function and parameters reported in Gómez-Schiavon, Dods et al. (2020) for the individual parts synthesis rate, it was observed that for a wide range of parameter values (η₊, heterodimer binding rate; μ_{Y}, YFP maximum synthesis rate; α_{Y}, YFP basal synthesis; K_{Y}, promoter dissociation constant).

FIG. 4A: the competition model

$$( {f}_{Y} = {μ}_{Y} \frac{{α}_{Y} {K}_{Y} + AD}{{K}_{Y} + AD + D} ) ,$$

but not the

$$no - competition = model ( {f}_{Y} = {μ}_{Y} \frac{{α}_{Y} {K}_{Y} + AD}{{K}_{Y} + AD} )$$

FIG. 4B displays the paradoxical effect observed in FIG. 1C: in all cases, increasing estradiol (E2), and in consequence the DBD part expression, reduces the output YFP expression compared to lower E2 concentrations. Three concentrations of E2 are shown: E2=72 nM (dark gray), E2=18 nM (gray), and E2=4.5 nM (light gray). Individual parameters-specified in each panel title-were varied from their nominal values (square markers) to a lower ten-fold value (×0.1, circle markers) and to a higher ten-fold value (×10, triangle markers). Used nominal parameter values: μ_{Y}=0.05 nM min⁻¹; α_{Y}=0.01; K_{Y}=0.02 nM; η₋=0.01 min⁻¹; n₊=0.05 nM⁻¹min⁻¹.

FIGS. 5A and 5B: Induced and uninduced all-by-all matrix values.

FIG. 5A: YFP expression values for the uninduced plate. Both species were given SDC media instead of inducers and measured 6 hours later. In both panels, designed interactions are outlined in red. In both, values represent the mean of 3 biological replicates.

FIG. 5B: YFP expression values for the induced plate. Both species were given saturating amounts of inducer (72 nM E2 and 128 nM Pg) and measured 6 hours later. Data were collected 6 hours after E2 and Pg induction. Values represent the mean of 3 biological replicates.

FIGS. 6A and 6B: The dominant negative (DN) model is highly sensitive to the relative expression of the DN part and high expression of DN is required to recapitulate the observed behavior. Using the synthesis function and parameters reported in Gómez-Schiavon, Dods et al. (2020) for the individual parts synthesis rate, except for the pZ3 promoter maximum synthesis rate μ_{P}

FIG. 6A: μ_{P}=0.00944 nM min⁻¹ as reported in Gómez-Schiavon, Dods et al.;

FIG. 6B: μ_{P}=0.0944 nM min⁻¹), the proposed model recapitulates the behavior observed in FIG. 2D-E. The specific shape of the dose response of increasing either the progesterone concentration (Pg) or the AD:DBD heterodimer binding rate (η₊) varies as the other unknown parameters change (β₊, either AD:DN or DBD: DN binding rate; μ_{Y}, YFP maximum synthesis rate; α_{Y}, YFP basal synthesis; K_{Y}, promoter dissociation constant). Three AD:DBD heterodimer binding rate (η₊) are shown: η₊=0.01 nM⁻¹min⁻¹ (dark gray), η₊=0.003 nM⁻¹min⁻¹ (gray), and η₊=0.001 nM⁻¹min⁻¹ (light gray). Individual parameters—specified in each panel title—were varied from their nominal values (square markers) to a lower ten-fold value (×0.1, circle markers) and to a higher ten-fold value (×10, triangle markers). Used nominal parameter values: μ_{Y}=0.05 nM min⁻¹; α_{Y}=0.05; K_{Y}=0.1 nM; η₋=0.01 min⁻¹; η₊={0.001,0.003,0.01} nM⁻¹min⁻¹; β₋=0.01 min⁻¹; β₊=0.05 nM⁻¹min⁻¹.

FIG. 7: The synthetic circuit does not confer a growth defect. Following induction for the steady-state cytometry experiment, a growth time-course was initiated. Colors represent the max concentration of the indicated Input (144 nM E2) and/or Pg (1024 nM) given to each circuit (column). WT represents the background strain with no circuit components, and the same data were plotted on each column for comparison. The faster growth observed for the circuit strains is most likely due to prototrophy conferred by the integration of the DNA constructs. Values represent the mean and s.d. of 3 biological replicates.

FIGS. 8A and 8B: Full steady-state dose responses.

FIG. 8A: Cartoon of the circuit.

FIG. 8B: Steady state responses of the Feedback and 2 No Feedback circuits, for all E2 and Pg values. All circuits were induced with the indicated values of E2 (columns) and Pg (x-axis), incubated for 8 hours, and measured for YFP and RFP fluorescence. In each column and row, the blue (YFP) or red (RFP) both represent the feedback circuit, while the greys represent the two No Feedback circuits. Lines were added between the Feedback Circuit values to aid visualization and do not represent fits. Values represent the mean and s.d. of 3 biological replicates.

FIGS. 9A-9D: The feedback circuit model recapitulates the experimental qualitative behavior, and suggests that the system response is highly sensitive to Z3PM transcription rate (PAD) value as well as the relative expression of the DN part.

FIGS. 9A-9C: Using the synthesis function and parameters reported in Gómez-Schiavon, Dods et al. (2020) for the individual parts synthesis rate, except for the pZ3 promoter maximum synthesis rate μ_{P}, μ_{P}=0.00944 nM min⁻¹ as reported in Gómez-Schiavon, Dods et al.

FIG. 9D pp=0.0944 nM min⁻¹), the proposed model recapitulates the behavior observed in FIG. 3B. The specific shape of the dose response of increasing the progesterone concentration (Pg) varies as the unknown parameters change (μ_{AD}, Z3PM maximum synthesis rate; α_{AD}, Z3PM basal synthesis; K_{AD}, promoter dissociation constant; η₊, AD:DBD heterodimer binding rate; β₊, AD:DN binding rate), displaying high sensitivity for the specific μ_{AD} and η₊ values. Three concentrations of E2 are shown: E2=144 nM (dark gray), E2=36 nM (gray), and E2=9 nM (light gray). Individual parameters—specified in each panel title—were varied from their nominal values (square markers) to a lower ten-fold value (×0.1, circle markers) and to a higher ten-fold value (×10, triangle markers). Used nominal parameter values: μ_{AD}=0.05 nM min⁻¹; α_{AD}=0.05; K_{AD}=0.1 nM; η₋=0.01 min⁻¹; η₊=0.001 nM⁻¹min⁻¹; β₋=0.01 min⁻¹; β₊=0.05 nM⁻¹min⁻¹; μ₀=0.0015 nM⁻¹min⁻¹.

### Definitions

The terms “synthetic,” “chimeric,” and “engineered” as used herein refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant” as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

A “constitutive promoter” is an unregulated promoter that allows for continual transcription of its associated gene. Examples of constitutive promoters include CMV, EF1a (elongation factor 1 alpha), SV40 (simian vacuolating virus 40), PGK1 (phosphoglycerate kinase), Ubc (ubiquitin C), beta actin, CAG (containing CMV enhancer, chicken beta actin promoter, and rabbit beta-globin splice acceptor), TRE (tetracycline response element), and CaMKIIa (Ca²⁺/calmodulin-dependent protein kinase II).

An “inducible promoter” is a regulated promoter that allows for the transcription of its associated genes only in the presence of a specific stimulus. Examples of inducible promoters include tetracycline-regulated promoter, steroid-regulated promoter, metal-regulated promoter, and estrogen receptor-regulated promoter.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein” used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

Polypeptides may be “non-naturally occurring” in that the entire polypeptide is not found in any naturally occurring polypeptide. It will be understood that components of non-naturally occurring polypeptides may be naturally occurring, including but not limited to domains (such as functional domains) that may be included in some embodiments.

A “vector” or “expression vector” is a replicon, such as plasmid, phage, virus, or cosmid, to which another DNA segment, i.e. an “insert,” may be attached so as to bring about the replication of the attached segment in a cell.

The terms “domain” and “motif,” used interchangeably herein, refer to both structured domains having one or more functions and unstructured segments of a polypeptide that, although unstructured, retain one or more functions. For example, a structured domain may encompass but is not limited to a continuous or discontinuous plurality of amino acids, or portions thereof, in a folded polypeptide that comprise a three-dimensional structure which contributes to a function of the polypeptide. In other instances, a domain may include an unstructured segment of a polypeptide comprising a plurality of two or more amino acids, or portions thereof, that maintains a function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208.

The term “binding” refers to a direct association between two molecules, due to, for example, covalent, electrostatic, hydrophobic, and ionic and/or hydrogen-bond interactions, including interactions such as salt bridges and water bridges.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

“Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “chimeric antigen receptor” and “CAR,” used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains.

The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero dimerization of the two portions of the split CAR is pharmacologically controlled (e.g., as described in PCT publication no. WO 2014/127261 and US Patent Application No. 2015/0368342, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5 (215): 215ra172; Glienke et al. Front Pharmacol (2015) 6:21; Kakarla & Gottschalk 52 Cancer J (2014) 20 (2): 151-5; Riddell et al. Cancer J (2014) 20 (2): 141-4; Pegram et al. Cancer J (2014) 20 (2): 127-33; Cheadle et al. Immunol Rev (2014) 257 (1): 91-106; Barrett et al. Annu Rev Med (2014) 65:333-47; Sadelain et al. Cancer Discov (2013) 3 (4): 388-98; Cartellieri et al., J Biomed Biotechnol (2010) 956304; the disclosures of which are incorporated herein by reference in their entirety. Useful CARs also include the anti-CD19-4-1BB-CD3z CAR expressed by lentivirus loaded CTL019 (Tisagenlecleucel-T) CAR-T cells as commercialized by Novartis (Basel, Switzerland). The terms “chimeric antigen receptor” and “CAR” also include SUPRA CAR and PNE CAR (see, e.g., Cho et al Cell 2018 173:1426-1438 and Rodgers et al, Proc. Acad. Sci. 2016 113: E459-468).

The terms “T cell receptor” and “TCR” are used interchangeably and will generally refer to a molecule found on the surface of T cells, or T lymphocytes, that is responsible for recognizing fragments of antigen as peptides bound to major histocompatibility complex (MHC) molecules. The TCR complex is a disulfide-linked membrane-anchored heterodimeric protein normally consisting of the highly variable alpha (a) and beta (b) chains expressed as part of a complex with CD3 chain molecules. Many native TCRs exist in heterodimeric ab or gd forms. The complete endogenous TCR complex in heterodimeric ab form includes eight chains, namely an alpha chain (referred to herein as TCRa or TCR alpha), beta chain (referred to herein as ‘H¾b or TCR beta), delta chain, gamma chain, two epsilon chains and two zeta chains. In some instance, a TCR is generally referred to by reference to only the TCRa and TCRb chains, however, as the assembled TCR complex may associate with endogenous delta, gamma, epsilon and/or zeta chains an ordinary skilled artisan will readily understand that reference to a TCR as present in a cell membrane may include reference to the fully or partially assembled TCR complex as appropriate.

Recombinant or engineered individual TCR chains and TCR complexes have been developed. References to the use of a TCR in a therapeutic context may refer to individual recombinant TCR chains. As such, engineered TCRs may include individual modified TCRa or modified TCRb chains as well as single chain TCRs that include modified and/or unmodified TCRa and TCRP chains that are joined into a single polypeptide by way of a linking polypeptide.

The terms “synthetic Notch receptor,” “synNotch,” and “synNotch receptor,” used interchangeably herein, refer to recombinant chimeric binding-triggered transcriptional switches that include at least: an extracellular binding domain, a portion of a Notch receptor that includes at least one proteolytic cleavage site, and an intracellular domain that provides a signaling function. SynNotch polypeptides, the components thereof and methods of employing the same, are described in U.S. Pat. Nos. 9,834,608 and 9,670,281, as well as, Toda et al., Science (2018) 361 (6398): 156-16; Roybal & Lim, Annu Rev Immunol. (2017) 35:229-253; Lim & June Cell. (2017) 168 (4): 724-740; Roybal et al. Cell. (2016) 167 (2): 419-432.el6; Roybal et al. Cell. (2016) 164 (4): 770-9; and Morsut et al. Cell. (2016) 164 (4): 780-91; the disclosures of which are incorporated herein by reference in their entirety.

The terms “exogenous” and “external” are used interchangeably herein to refer to a stimulus that is initiated outside of a cell that is transduced or moves to the inside of a cell. Molecules that are added to the outside of a cell that are cross the plasma membrane to the inside of the cell are one type of stimulus. Another type of stimulus is a signal transduction event that crosses the plasma membrane. For example, binding of a receptor on the cell to a ligand or antigen on another cell is another type of stimulus, where the binding causes a signal to be transduced to the inside of the cell.

The term “split protein” refers to a protein that is split into two parts. When the parts are brought together i.e., “reconstituted” (e.g., via a dimerization domain), the protein has an activity that the parts don't have individually. For example, one part of a split transcription factor typically contains DNA-binding domain and whereas the other part has the activating domain. Together these parts activate transcription. Several other examples exist in the literature, e.g., enzymes, etc.

The term “designed heterodimer” refers to the helix-loop-helix bundles described in Chen et al (Nature 2019 565:106-111) and US20210355175A1.

Before the present invention is further described, it is to be understood that this invention is not limited to embodiments described, as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a circuit” includes a plurality of such circuits and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The disclosure relates to molecular circuits, cells comprising such molecular circuits and methods of using the cells in controlling cellular behaviors, for example, in controlling cellular behavior in cellular therapies.

In first embodiments, the cell may comprise a molecular switch comprising: (a) a first polypeptide comprising a first part of a split protein and a first monomer of a designed heterodimer; (b) a second polypeptide comprising a second part of a split protein and a second monomer of a designed heterodimer; and (c) a third polypeptide comprising a third monomer of a designed heterodimer, but neither parts of the split protein.

In some of these embodiments, (a) and (b) may bind to each other via a relatively low affinity interaction between their monomers to produce a reconstituted split protein that has an activity that is not provided by either (a) or (b) alone; and (c) may bind to (a) and/or (b) via a relatively high affinity between their monomers, thereby inactivating the reconstituted split protein. As noted below, the high affinity monomer displaces the low affinity monomer, thereby disassociating the (a) and (b). Because the third polypeptide of (c) does not contain either of the parts of the split protein, the split protein is inactivated.

In other of these embodiments, (a) and (c) bind to each other via a relatively low affinity interaction between their monomers; and (b) binds to (a) via a relatively high affinity between their monomers to produce a reconstituted split protein that has an activity is not provided by either (a) or (b) alone. In this case, the high affinity monomer displaces the low affinity monomer, thereby bringing together (a) and (b) to reconstitute the split protein.

In second embodiments, the cell may comprise a feedback circuit comprising: (a) a first polypeptide comprising a first part of a split protein and a monomer of a designed heterodimer; (b) a second polypeptide comprising a second part of a split protein and a monomer of a designed heterodimer; and (c) a third polypeptide comprising a monomer of a designed heterodimer, not containing the first or second parts of the split protein. In these embodiments, (a) and (b) may bind to each other via an interaction between their monomers to produce a reconstituted split protein that has an activity is not provided by either (a) or (b) alone, and (c) binds to (a) or (b) via an interaction between their monomers, thereby inactivating the reconstituted split protein (by displacing the polypeptide that it does not bind to). In these embodiments, expression of (c) is regulated by the activity of the reconstituted split protein, which makes it a feedback circuit in which the expression of (c) is regulated by its own expression.

In third embodiments, the cell may comprise a feedforward circuit comprising: (a) a first polypeptide comprising a first part of a split protein and a monomer of a designed heterodimer; (b) a second polypeptide comprising a second part of a split protein and a monomer of a designed heterodimer; (c) a third polypeptide comprising a monomer of a designed heterodimer, not linked to the first or second parts of the split protein, and (d) an actuating protein that, in response to an external stimulus, independently activates expression of (c) and at least one of (a) and (b) In these embodiments: (a) and (b) may bind to each other via an interaction between their monomers to produce a reconstituted split protein that has an activity is not provided by either (a) or (b) alone; and (c) may bind to (a) or (b) via an interaction between their monomers, thereby inactivating the reconstituted split protein (by displacing the polypeptide that it does not bind to).

In any of the second and third embodiments, (a) and (b) may bind to each other via a relatively low affinity interaction; and (c) binds to (a) or (b) via a relatively high affinity interaction.

In any embodiment, the difference between the affinities (i.e., the difference in the affinities of high and low affinity binding interactions, e.g., the differences in affinities between (c) and (a) or between (c) and (b) may be at least a 2×, at least a 5×, at least a 10×, or at least a 100× difference, as measured by any suitable assay (e.g., Biocore).

In some embodiments (e.g., the second and third embodiments), the monomer of (c) may be the same as the monomer of (a) or (b). However, in other embodiments the monomer of (c) may be different to the monomer of (a) or (b).

In some embodiments, expression of (a) and/or (b) may be activated by an external stimulus, e.g., an exogenously added drug (e.g., an estrogen or prostaglandin) or a binding event on the cell surface that is transduced to the inside of the cell, where the term “activated” is intended to mean that the expression of (a) and/or (b) is induced by the stimulus, or the protein undergoes a conformational change or binding event that activates it. For example, as shown in the examples section of this disclosure, expression of (a) and/or (b) can be under the control of an inducible promoter that can be induced by an exogenous compound or a signal transduction event.

In the third embodiments, the cell may further comprise (e) a controller protein that regulates the interaction between the (c) and (a) or (b), e.g., by binding to or inactivating one of the proteins, or blocking the interaction between two of the proteins. The controller protein can, itself, can contain a monomer of a designed heterodimer, thereby allowing it to interact with the other polypeptides. To add further control, the expression and/or activity of the controller protein may be modulated by a second exogenous stimulus.

In any embodiment, the third polypeptide may comprise a degron. In these embodiments, the third polypeptide may comprise (in addition to the monomer of the designed heterodimer) a ubiquitination-recruiting domain (e.g., a degron or an E3 ligase-recruiting domain that binds directly or indirectly (via an adapter protein) to an E3 ligase), where binding of the third polypeptide to another polypeptide via the monomer induces degradation of the other protein via the ubiquitination-mediated degradation. Examples of such proteins (which should be lysine-free) that could potentially be employed are described in PCT/US2021/47391 filed Aug. 24, 2021, and others. For example, in some embodiments, the third polypeptide may contain C-terminal degron sequence (e.g., RRRG (SEQ ID NO: 299); also referred to as the “Bonger” motif). This polypeptide may be lysine-free and targets other proteins for degradation in trans (i.e., by binding to them).

The monomers may have any suitable structure, as long as they heterodimerize orthogonally. In some embodiments, the monomers may contain a dimerization domain such as a heterospecific coiled-coil interaction domain, which domains may be referred to as synthetic leucine zipper domains (“synZIPs”) in certain publications (see, e.g., Potapov et al (PLOS Comput Biol 11 (2): e1004046) and Thompson et al. (ACS Synth Biol. 2012 1:118-129)). These dimerization domains have a coiled-coil interaction domain. In many embodiments, however, the monomers of the designed heterodimer may be selected from any of SEQ ID NOS: 1-298 (see also US20210355175), or a variant thereof that has at least 90% or at least 95% identity to any of those sequences. In some embodiments, a variant may be shorter (e.g., by up to 5 amino acids or up to 10 amino acids) relative to any of those sequence. Because these sequences are designed computationally (see, e.g., Chen et al (Nature 2019 565:106-111)), variants can be designed. In this listing, each pair of sequences (1 and 2, 3 and 4) etc., has a high affinity interaction. Based on the results below, many of monomers will have lower affinity interactions with other monomers, thereby facilitating some of the embodiments described herein. In any embodiment, the monomers may be selected from SEQ ID NOS: 291-298) or a variant thereof that has at least 90% or at least 95% identity to any of those sequences, data for which may be found in the experiment section of this patent application:

DHD37A:
(SEQ ID NO: 291)
DSDEHLKKLKTFLENLRRHLDRLDKHIKQLRDILSENPEDERVKDVIDL
SERSVRIVKTVIKIFEDSVRKKEGSEGSGSEGS
DHD37B:
(SEQ ID NO: 292)
GSDDKELDKLLDTLEKILQTATKIIDDANKLLEKLRRSERKDPKVVETY
VELLKRHEKAVKELLEIAKTHAKKVEGSEGSGSEGS
DHD13A:
(SEQ ID NO: 293)
GTKEDILERQRKIIERAQEIHRRQQEILEELERIIRKPGSSEEAMKRML
KLLEESLRLLKELLELSEESAQLLYEQRGSEGSGSEGS
DHD13B:
(SEQ ID NO: 294)
GTEKRLLEEAERAHREQKEIIKKAQELHRRLEEIVRQSGSSEEAKKEAK
KILEEIRELSKRSLELLREILYLSQEQKGSEGSGSEGS
DHD154A:
(SEQ ID NO: 295)
TAEELLEVHKKSDRVTKEHLRVSEEILKVVEVLTRGEVSSEVLKRVLRK
LEELTDKLRRVTEEQRRVVEKLN
DHD154B:
(SEQ ID NO: 296)
DLEDLLRRLRRLVDEQRRLVEELERVSRRLEKAVRDNEDERELARLSRE
HSDIQDKKDKLAREILEVLKRLLERTE
DHD155A:
(SEQ ID NO: 297)
PEDDVVRIIKEDLESNREVLREQKEIHRILELVTRGEVSEEAIDRVLKR
QEDLLKKQKESTDKARKVVEERR
DHD155B:
(SEQ ID NO: 298)
DEVRLITEWLKLSEESTRLLKELVELTRLLRNNVPNVEEILREHERISR
ELERLSRRLKDLADKLERTRR

In any embodiment, the reconstituted split protein may be a transcription factor, enzyme, kinase or a cell-surface receptor (e.g., a CAR), for example. In some embodiments, the split protein may a split transcription factor. In these embodiments, the cell may comprise an expression cassette comprising a promoter and a coding sequence that are operably linked, and the reconstituted transcription factor binds to the promoter and activates transcription of the coding sequence. Such a system could contain transcription factor could have the DNA binding domain of GAL4, a viral activation domain (e.g., the VP16 activation domain) and a UAS sequence, although several alternatives are possible. The coding sequence whose transcription is induced by the reconstituted transcription factor may encode in immune receptor, (e.g., a TCR or CAR), cytokine or enzyme. In some embodiments, the coding sequence may encode a therapeutic protein that, when expressed, may secreted by the cell or may be on the surface of the cell. In embodiments in which the therapeutic protein is secreted, the therapeutic protein may be, for example, an antibody (e.g., an antibody that binds to PD1, PD-L1, PD-L2, CTLA4, TIM3 or LAG3 or another immune checkpoint, for example), an enzyme (e.g., a superoxide dismutase for removing reactive oxygen species or a protease that can unmask a probody) or a bioactive peptide such as a cytokine (e.g., Il-1ra, IL-4, IL-6, IL-10, IL-11, IL-13, or TGF-β, among many others). In other embodiments, the coding sequence may encode an industrial enzyme, for example.

Suitable cells include stem cells, progenitor cells, as well as partially and fully differentiated cells. Suitable cells include, neurons, liver cells; kidney cells; immune cells; cardiac cells; skeletal muscle cells; smooth muscle cells; lung cells; and the like.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs).

In some instances, a cell is an immune cell. Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual. As another example, the cell is a stem cell or progenitor cell obtained from an individual.

As used herein, the term “immune cells” generally includes white blood cells (leukocytes) which are derived from hematopoietic stem cells (HSC) produced in the bone marrow. “Immune cells” includes, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses. “B cell” includes mature and immature cells of the B cell lineage including e.g., cells that express CD19 such as Pre B cells, Immature B cells, Mature B cells, Memory B cells and plasmablasts. Immune cells also include B cell progenitors such as Pro B cells and B cell lineage derivatives such as plasma cells.

Cells encoding a molecular circuit of the present disclosure may be generated by any convenient method. Nucleic acids encoding one or more components of a molecular circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, an immune cell, a stem cell, etc., is obtained from an individual; and the cell obtained from the individual is modified to express components of a circuit of the present disclosure. The modified cell can thus be modified with one or more signaling pathways of choice, as defined by the one or more molecular circuits present on the introduced nucleic acids. In some cases, the modified cell is modulated ex vivo. In other cases, the cell is introduced into (e.g., the individual from whom the cell was obtained) and/or already present in an individual; and the cell is modulated in vivo, e.g., by administering a nucleic acid or vector to the individual in vivo. For example, in some embodiments, nucleic acid encoding the current proteins (e.g., mRNA) can be delivered in vivo, e.g., using T cell-targeted lipid nanoparticles (LNPs).

In some instances, cells employing a molecular circuit of the present disclosure may be therapeutic cells useful in cellular therapy of a subject. For example, in an application such as cellular therapy employing immune cells, the immune cells are engineered to deliver a therapeutic payload of interest in the human body. If the output of these engineered cells is too high, toxic effects may occur (such as e.g., cytokine release syndrome (CRS) as observed in CAR-T cell therapies), but on the other hand an output that is too low then the therapy may be ineffective. Therapeutic cells can be fine-tuned to achieve a desired level of output (i.e., a setpoint) under well-controlled laboratory conditions. However, the dynamic environments in which engineered therapeutic cells function make guaranteeing that the output will remain constant over time difficult. Using the molecular circuits described herein for implementing feedback control, engineered cells can automatically correct against disturbances encountered the environment, including e.g., disturbances that cause the output to drift. In one aspect, self-regulating engineered cells are more robust in in vivo scenarios, thus improving existing cell therapy applications of synthetic biology.

In some instances, cellular therapeutics such as CAR-T cells or synthetic receptor (e.g., SynNotch) enabled T cells greatly benefit from control as a safety mechanism. A molecular circuit in a CAR-T cell may regulate the level of T cell activation and prevent toxic effects such as CRS which result from overstimulation of immune cells. Similarly, in other cases a molecular circuit may enable delivery of a precise concentration of a payload of interest regardless of any disturbances to the engineered cell that are present or introduced. In certain embodiments, the cells can be engineered to provide a self-limited therapeutic action in response to a stimulus (e.g., a pulse of activity).

Circuits and/or methods of the present disclosure may be used in conjunction with several different production techniques known in the art, such as the production of biological products using cells in a bioreactor (e.g., mammalian, yeast, bacteria, and/or insect cells), methods involving the use of transgenic animals (e.g. goats or chickens), methods involving the use of transgenic plants (e.g., tobacco, seeds or moss), and other methods known to those of skill in the art.

In some instances, molecular circuits are employed for metabolic engineering, where extended expression of an intermediate or constitutive expression of this intermediate without input is detrimental. It is common for intermediates or even final products of metabolic pathways to have at least some level of toxicity to the host cell. Therefore, optimization of their expression dynamics in pulses or only as certain other intermediate are at certain concentration levels is beneficial to maximizing the amount of product produced while maintaining effective cell growth.

Nucleic acids encoding the present system are also disclosed. Cells comprising nucleic acid encoding the molecular switch, feedback circuit or feedforward circuit are also provided. Because the genetic code is known, nucleic acids encoding the present system can be readily derived given the description of the proteins. In some instances, the subject circuits may make use of an encoding nucleic acid (e.g., a nucleic acid encoding a target protein) that is operably linked to a regulatory sequence such as a transcriptional control element (e.g., a promoter; an enhancer; etc.). In some cases, the transcriptional control element is inducible. In some cases, the transcriptional control element is constitutive. In some cases, the promoters are functional in eukaryotic cells. In some cases, the promoters are functional in prokaryotic cells. In some cases, the promoters are cell type-specific promoters. In some cases, the promoters are tissue-specific promoters Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

The present disclosure also provides a method for regulating gene expression that uses the cell described above, i.e., a cell that has been genetically modified with a molecular switch or circuit as described above. In some embodiments, the method may comprise exposing the cell to the external stimulus, thereby actuating the switch, feedback control loop or feedforward control loop. This method may be done in vivo, ex vivo, or in vitro.

For example, in some instances, a circuit of the present disclosure may be employed in a method to provide control of a signaling pathway in response to an exogenous stimulus. In some instances, molecular circuit may include a switch, feedback control or feedforward control, which may, among other aspects, e.g., prevent the pathway from remaining active when a pathway output is produced and/or produced at or above a threshold level. In some instances, molecular circuit may include positive feedback control or feedforward control, which may, among other aspects, e.g., provide for amplification of a pathway output. In some instances, a molecular circuit may provide for more stable output of a signaling pathway, including e.g., where the signaling output of the pathway is insulated from variables such as but not limited to e.g., environmental factors and inputs.

Cells of the methods of the present disclosure may vary and may include in vitro and/or ex vivo cells genetically modified with one or more nucleic acids encoding one or more components of one or more circuits as described herein. In some instances, cells are primary cells obtained from a subject. In some instances, cells are obtained from a cell culture.

Accordingly, methods of the present disclosure may include obtaining cells used in the method, including where such cells are unmodified or have already been genetically modified to include a molecular circuit of the present disclosure. In some instances, methods of the present disclosure may include performing the genetic modification. In some instances, methods of the present disclosure may include collecting cells, including where cells are collected before and/or after genetic modification. Methods of collecting cells may vary and may include e.g., collecting cells from a cell culture, collecting a cellular sample from a subject that includes the cells of interest, and the like.

Once the molecular circuit is initiated and/or a cell containing the molecular circuit is delivered, modulation of the signaling pathway in accordance with the molecular circuit may not necessitate further manipulation, i.e., regulation of the signaling pathway by the molecular circuit may be essentially automatic.

Accordingly, in certain methods employing cells that contain a molecular circuit of the present disclosure, the cells may be administered to the subject and no further manipulation of the molecular circuit need be performed. For example, where a subject is treated with cells that contain a molecular circuit of the present disclosure, the treatment may include administering the cells to the subject, including where such administration is the sole intervention to treat the subject.

In such methods, cells that may be administered may include, but are not limited to e.g., immune cells. In such methods, the molecular circuit may be configured, in some instances, to modulate signaling of a native or synthetic signaling pathway of the immune cell, such as but not limited to e.g., an immune activation pathway or an immune suppression pathway. Non-limiting examples of suitable immune activation pathways, whether regulated by native or synthetic means, include cytokine signaling pathways, B cell receptor signaling pathways, T cell receptor signaling pathways, and the like. Non-limiting examples of suitable immune suppression pathways, whether regulated by native or synthetic means, include inhibitory immune checkpoint pathways, and the like.

Methods of the present disclosure may include administering to a subject the cells that express a therapeutic agent. Such cells may include a molecular circuit of the present disclosure and may or may not be immune cells. For example, in some instances, a method may include administering to a subject a non-immune cell that produces a therapeutic agent, either endogenously or heterologously, where production of the therapeutic is controlled, in whole or in part, by the molecular circuit. In some instances, a method may include administering to a subject an immune cell that produces a therapeutic agent, either endogenously or heterologously, where production of the therapeutic is controlled, in whole or in part, by the molecular circuit. Non-limiting examples of suitable encoded therapeutic agents, include but are not limited to e.g., hormones or components of hormone production pathways, such as insulins or a component of an insulin production pathway, estrogen/progesterone or a component of an estrogen/progesterone production pathway, testosterone or a component of an androgen production pathway, growth hormone or component of a growth hormone production pathway, or the like.

Such methods may be employed, in some instances, to treat a subject for a condition, including e.g., where the condition is a deficiency in a metabolic or a hormone. In such instances, the molecular circuit may be configured such that the output of the molecular circuit controls, in whole or in part, production and/or secretion of a metabolic or a hormone.

Methods of the instant disclosure may further include culturing a cell genetically modified to encode a molecular circuit of the instant disclosure including but not limited to e.g., culturing the cell prior to administration, culturing the cell in vitro or ex vivo (e.g., the presence or absence of one or more antigens), etc. Any convenient method of cell culture may be employed whereas such methods will vary based on various factors including but not limited to e.g., the type of cell being cultured, the intended use of the cell (e.g., whether the cell is cultured for research or therapeutic purposes), etc. In some instances, methods of the instant disclosure may further include common processes of cell culture including but not limited to e.g., seeding cell cultures, feeding cell cultures, passaging cell cultures, splitting cell cultures, analyzing cell cultures, treating cell cultures with a drug, harvesting cell cultures, etc.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLES

Dynamic dimerization is a common regulatory interaction between biological molecules, underpinning many signaling functions. Because of its ubiquity, many biological engineering efforts have focused on building dimerizing proteins, such as the SYNZIPs and de novo Designed HeteroDimers. Using the Designed HeteroDimers as a model system, it has been shown that low-affinity protein interactions can be competitively displaced by a high-affinity “dominant negative” heterodimer. The utility of this signaling motif has been demonstrated using competitive displacement to implement negative feedback in a synthetic circuit. Competitive displacement could be extended to other heterodimer systems to expand the functionality of protein circuits and enable new biotechnological and therapeutic applications. In other words, it has been demonstrated that the binding of the dimerized partners can be reversed. Specifically, it has been shown that dominant negative heterodimers can competitively displace weaker interactions to implement negative regulation of split proteins. In this study, a subset of the DHD library was characterized using a titratable^{27.28} split transcription factor (TF) system. Using a selection of four DHD pairs, all possible combinations of interactions between individual DHD monomers were studied to identify identify several novel low-affinity, off-target interactions. It has been shown that these low-affinity interactions can be competitively displaced by introducing the on-target, high-affinity heterodimer, referred to here as the “dominant negative.” The properties of the dominant negative and dynamics of competitive displacement are characterized and this information was used to design a negative feedback circuit that utilizes a dominant negative to implement negative regulation. The application of competitive displacement to control other split proteins offers a promising strategy that could greatly expand the scope of protein circuits for a variety of biotechnological and therapeutic applications.

### Material and Methods

Media: Overnight yeast cultures were grown in YPD (1% w/v bacto-yeast extract; 2% w/v bacto-peptone; and 2% w/v dextrose). Yeast transformation cultures were diluted in YPD. Cultures for flow cytometry experiments were diluted in SDC (0.67% w/v Difco yeast nitrogen base without amino acids; 0.2% complete supplement mixture (MP Biomedicals); and 2% w/v dextrose). SDC agar plates with the appropriate nutrient removed (Teknova) were used for selection after transformation.

Plasmid and Strain Construction: All plasmids were constructed using the Yeast Toolkit standard for hierarchical Golden Gate assembly³⁸. The enzymes BsaI-HF v2 (NEB), T4 DNA ligase (NEB), and Esp3I FastDigest (Thermo Fisher Scientific) were used for these reactions. Sequences for the Designed HeteroDimers were provided by Zibo Chen and the Baker lab and ordered as gBlocks (IDT)²⁶. Sequences for the SynTF ZF43_8 were provided by the Khalil lab and PCR amplified using Q5 High-Fidelity 2× Master Mix (NEB)¹⁴. All DNA manipulations were performed with standard molecular biology techniques.

Flow Cytometry Experiments: Yeast strains were streaked out onto YPD plates from glycerol stocks, except for the all-by-all matrix experiment, where three transformation colonies were tested. Individual colonies were picked into 1 mL of YPD in a 2-mL V-bottom 96-well block (Corning/Costar) for overnight growth at 30° C. and 900 rpm in a Multitron shaker (Infors HT). Following overnight growth, strains were diluted 1:500 in SDC and 400 μL and then were aliquoted into a new 96-well block for a two hour outgrowth. For the all-by-all matrix, cultures were diluted 1:200 in SDC.

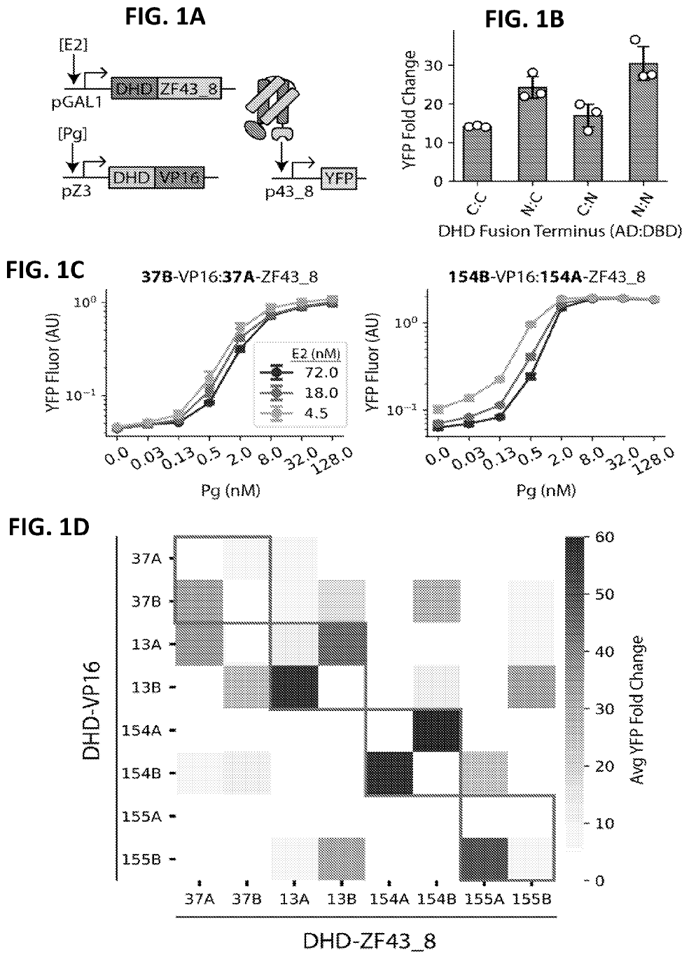

For the termini fusion experiment (FIG. 1B), dilutions were done in 12 mL SDC in an 8-row block and aliquoted into the rows of a 96-well block. For the 2D inductions of individual interactions (FIG. 1C), dilutions were done in 15 mL in a 50 mL trough (Corning) and aliquoted across the rows of the 96-well block. For the all-by-all matrix experiment (FIG. 1D, S2), dilutions were done in 400 uL in a new 96-well block.

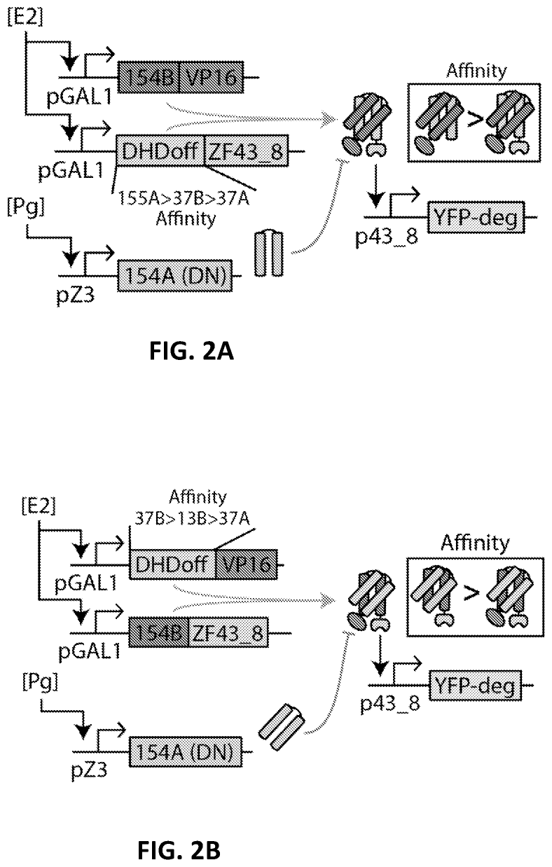

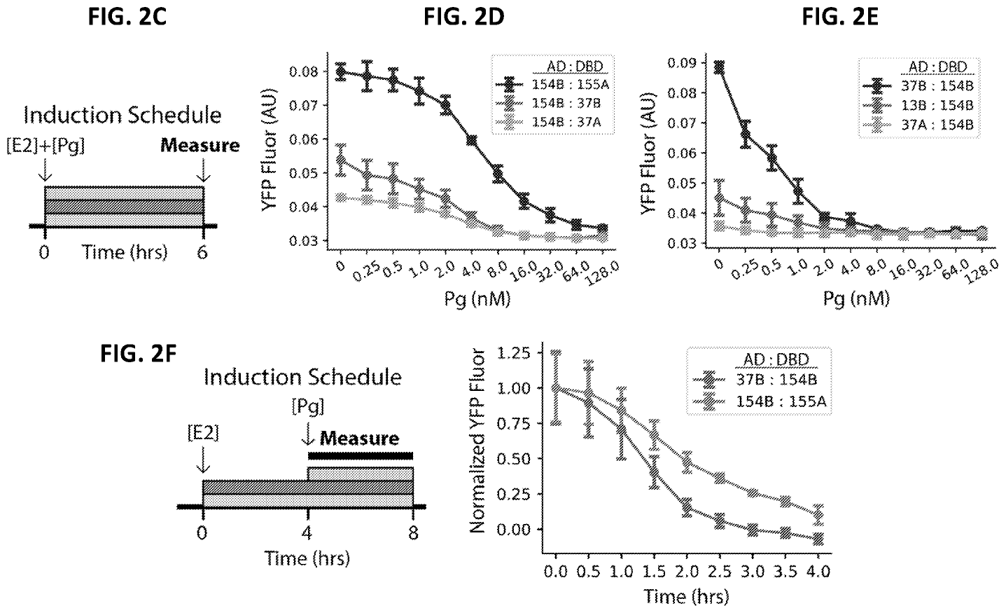

For the competition and reversibility assays (FIG. 2), dilutions were done in 12 mL in an 8-row block and aliquoted across the rows of the 96-well block.

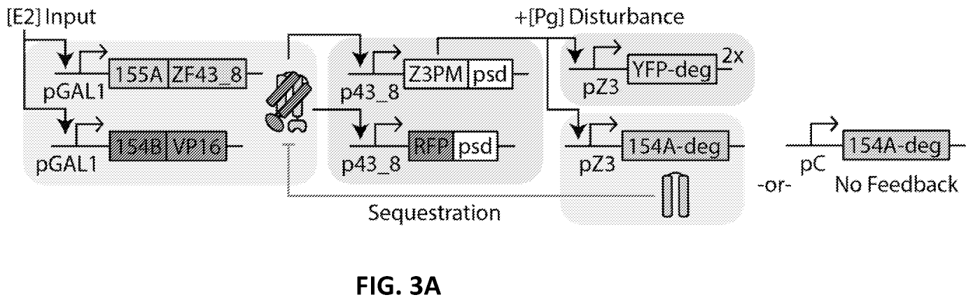

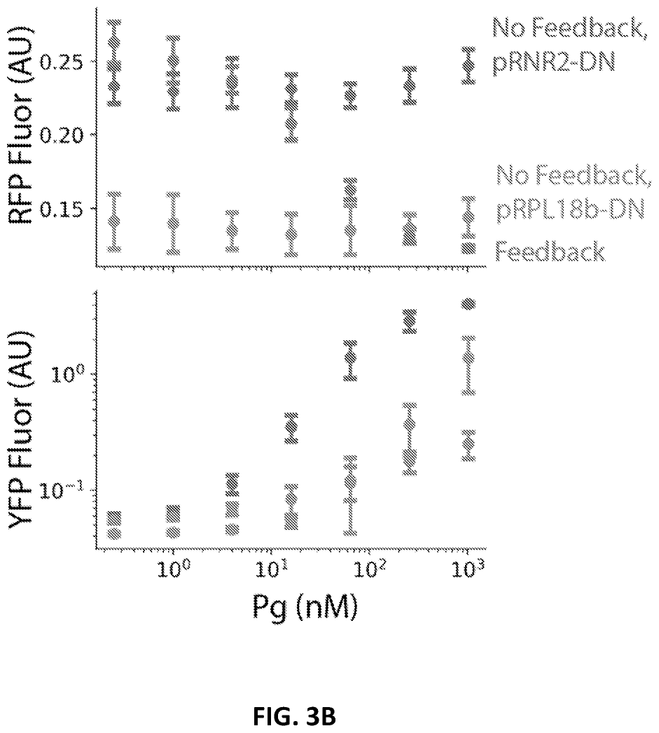

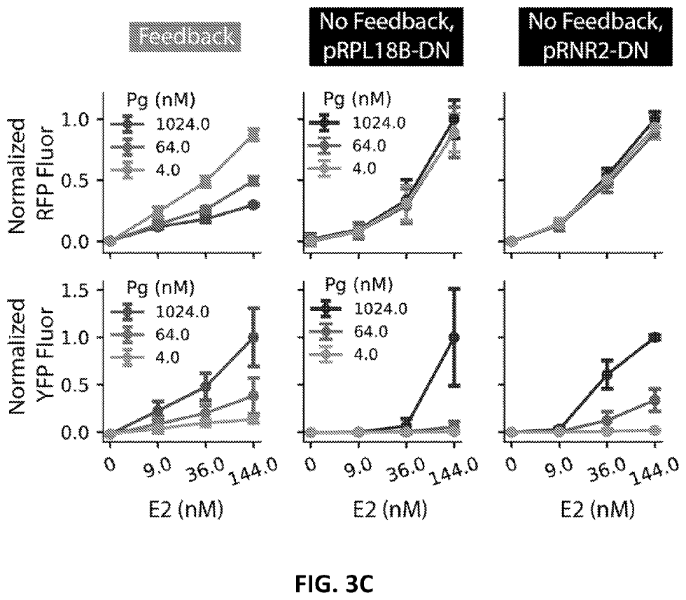

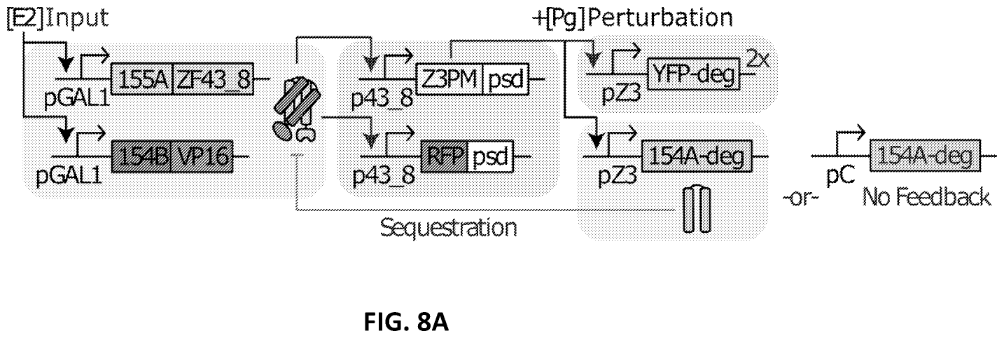

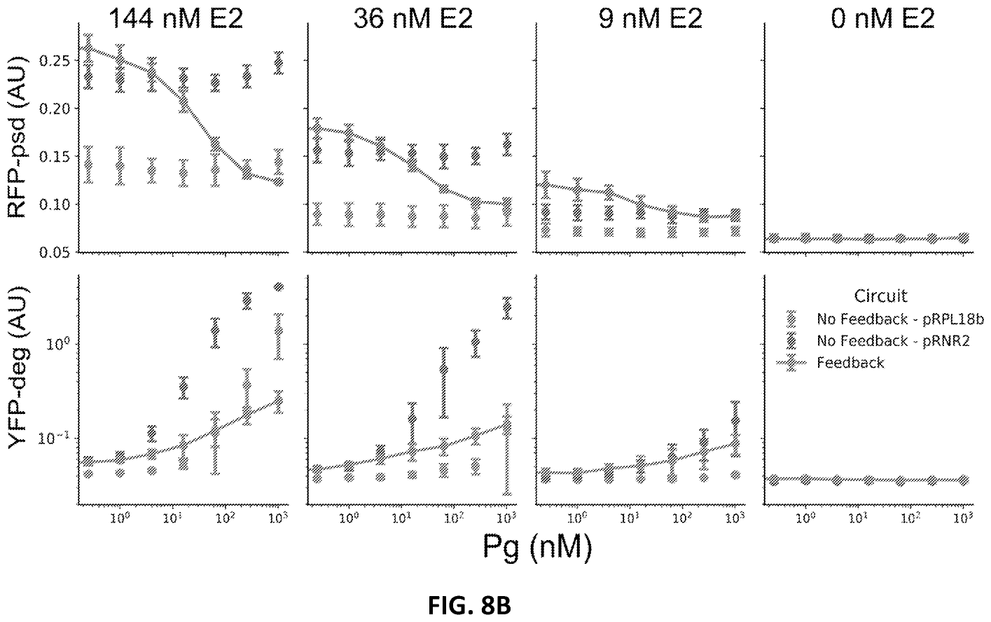

For the feedback experiments (FIGS. 3, 8), dilutions were done in 45 mL in a 50 mL trough and aliquoted across the rows of the 96-well block.

During the 2 hour outgrowth, estradiol (Sigma-Aldrich) and progesterone (Fisher Scientific) induction gradients were prepared. Ten-times concentrated solutions were made in fresh SDC from 36 micromolar (estradiol) and 32 micromolar (progesterone) stock solutions. Gradients were either one-to-one (FIG. 2) or one-to-three (FIG. 1C, 3) serial diluted from a maximum induction solution. After the 2 hour outgrowth, 50 microliters of the corresponding solution were added to the appropriate wells at the appropriate times. For all experiments except those in FIG. 2F, both solutions were added at the same time, and then blocks were returned to the shaker until measurement. For FIG. 2F, the E2 solutions were added after the outgrowth, and the blocks were returned to the shaker for a 4 hour activation period. Then, the Pg solutions were added to induce the dominant negative.

For the orientation experiment (FIG. 1), inductions were incubated for 6 hours before measurement. For the all-by-all matrix experiment, inductions were incubated for 6 hours. For experiments in FIG. 2D-E, inductions were incubated for 6 hours. For experiments in FIG. 2F, Pg inductions were incubated for 4 hours. For the dynamics in FIG. 2F, inductions were measured every 30 minutes for 4 hours beginning immediately after induction of the DN. For the feedback experiments, inductions were incubated for 7.5-8.5 hours.

Following the requisite induction times, the cultures were prepared for flow cytometry. One hundred microliters of culture were mixed with 100 microliters of fresh SDC in a 96-well U-bottom microplate (greiner bio-one). Samples were measured on a BD LSRFortessa X20 (BD Biosciences) using a high-throughput sampler. YFP/Venus fluorescence was measured using the FITC-H channel (voltage=473). RFP/mScarlet fluorescence was measured using the PE-CF594-H channel (voltage=709). Measurements were normalized by dividing by SSC-H (voltage=192). For the feedback circuit experiments, compensation was performed with the FACSDiva software using a YFP benchmark strain (yAHN184) and RFP benchmark strain (yAHN642).

Analysis was performed with Python 3.7 and custom scripts using the FlowCytometryTools package. All experiments were performed with biological triplicates. Reported values represent the mean and standard deviation of median normalized fluorescence values of the individual replicate populations. For the experiments where fluorescence values are normalized to an intra-experiment max (FIG. 2F, 3C), background fluorescence was subtracted out and the resultant values were divided by the indicated max of the experiment.

Growth Assay: Measurement of growth was performed in parallel with the steady state measurements of the feedback circuit. Immediately following induction, 200 uL aliquots of each circuit with the indicated inducers (144 nM E2 and/or 1024 nM Pg), along with the background (WT) strain, were transferred to a Costar polystyrene, tissue culture treated 96 well assay plate with a clear flat bottom (Corning 3904) and sealed with a Breathe-Easy sealing membrane. Cultures were placed in the middle of the plate to avoid edge effects. The plate was then placed in a Spark 10M plate reader and Optical Density 600 nm (OD600) was measured every 30 minutes for 24 hours (Spark Control v2.2). The cultures were maintained at 30 C with double orbital shaking.

Abbreviations: AD—Activation Domain; DBD—NA Binding Domain; DHD—Designed HeteroDimer; DN—Dominant Negative; E2—estradiol; GEM—Gal4 DBD, Estradiol ligand binding domain, Msn2 AD; Pg—progesterone; RFP—red fluorescent protein; TF—transcription factor; YFP—yellow fluorescent protein; Z3PM-Z3 DBD, Progesterone ligand binding domain, Msn2 AD

### Results

Previous characterization of the designed heterodimers (DHDs) was carried out using a yeast two-hybrid system. Canonical yeast two-hybrid systems detect protein-protein interactions via dimerization-dependent activation of an auxotrophic marker. Although this assay is useful for rapidly screening protein interaction pairs, colony formation or cell growth is typically binary and does not necessarily inform relative binding affinity. The rational design of protein-based circuits requires a quantitative understanding of relative protein affinities in a cellular context. To achieve this, \ a fluorescence-based split transcription factor (TF) assay was developed in S. cerevisiae. DHDs were fused to either a synthetic zinc-finger ZF43_8 DNA-binding domain (DHD-DBD or DHD-ZF43_8) or VP16 activation domain (DHD-AD or DHD-VP16).¹⁴ Interaction between the DHDs reconstitutes the ZF43_8-VP16 transcription factor and activates the cognate p43_8 promoter to drive expression of a yellow fluorescent protein (YFP) reporter (FIG. 1A). Two orthogonal drug-inducible systems (GEM and Z3PM)^{27,28} were utilized to express different levels of the DHD-DBD fusion and the DHD-AD fusion. The GEM synthetic transcription factor is induced by estradiol (E2) to activate the pGAL1 promoter and produce the DHD-DBD fusion. The Z3PM synthetic transcription factor is activated by the orthogonal drug progesterone (Pg) to induce the pZ3 promoter and produce the DHD-AD fusion.

The optimal orientation for fusing a DHD to either ZF43_8 or VP16 was investigated. The 37B monomer was fused to VP16 and the cognate 37A monomer to ZF43_8 and the YFP expression of all four combinations of N- and C-terminal fusions were measured. Expression of the DBD species was saturated and YFP expression was measured in the presence and absence of the AD species. The largest dynamic range was observed when fusing both DHDs to the N-termini of VP16 and ZF43_8 (FIG. 1B). Given the common structure of the DHDs, it is assumed that the N-terminal fusions would be optimal for all other DHDs and thus used this configuration for all other experiments.

To test the full dynamic range of the assay, a two-dimensional induction was performed where the expression of the DHD-ZF43_8 and DHD-VP16 fusions were independently titrated for both the 37 and 154 DHD on-target pairs (FIG. 1C). In the absence of both inducers, minimal YFP expression was observed. As expected, YFP expression increased with increasing amounts of Pg (DHD-VP16). Interestingly, for low to intermediate concentrations of Pg (DHD-VP16), YFP expression decreased with increasing amounts of E2 (DHD-ZF43_8). YFP expression was insensitive to increasing E2 at high concentrations of Pg. This paradoxical effect may be a result of excess, free DHD-ZF43_8 acting as a transcriptional repressor by competing for promoter occupancy with the bound DHD-ZF43_8: DHD-VP16 complex.

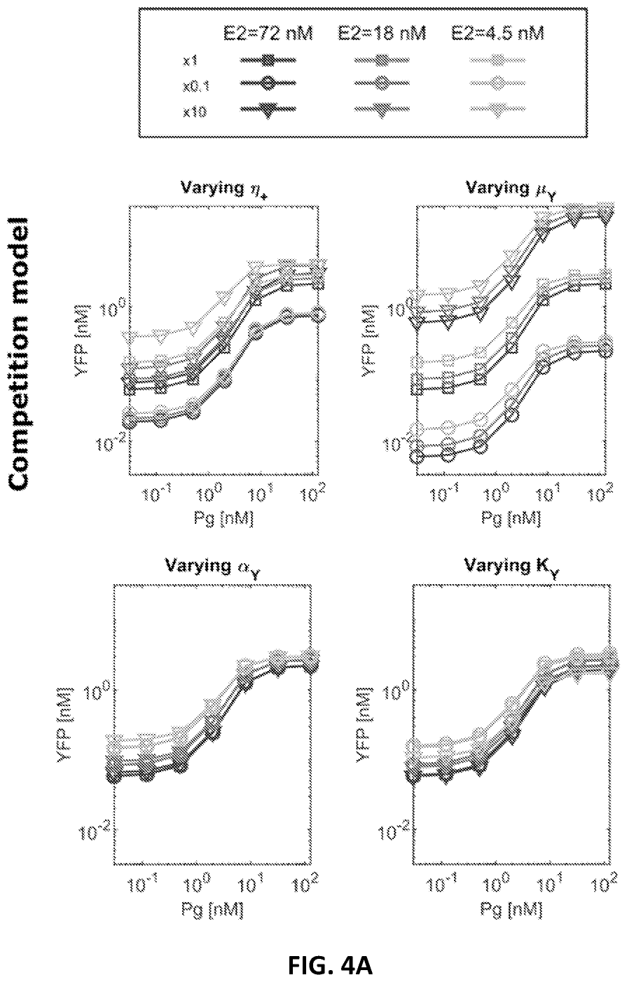

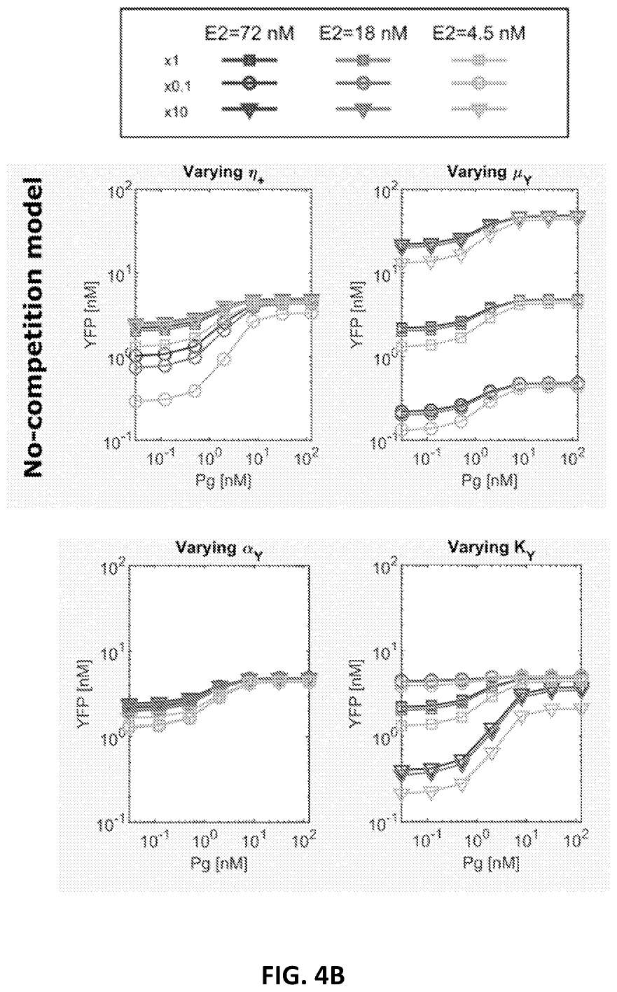

To explore this hypothesis, a simple promoter occupancy competition model was proposed based on the system in FIG. 1A. This model considers that the regulated promoter can exist in three states: unbound/free (with basal transcriptional activity inherent to the promoter), bound to the DHD TF (with full transcriptional activity), or bound to the free DBD species (without transcriptional activity). In the competition model, the amount of free DBD species effectively represses the transcriptional activity by competing for the promoter binding site (FIG. 4A). For comparison, a no-competition model was also built in which the effect of the free DBD species on promoter transcriptional activity is assumed to be negligible and is excluded from the model (i.e. only two promoter states, unbound/free and bound to the DHD TF, are considered; FIG. 4B). For a wide range of parameter values, the competition model displays the paradoxical effect observed in FIG. 1C: increasing the DBD part expression by increasing estradiol (E2) concentration reduces the YFP reporter expression for low or medium progesterone (Pg) concentrations (FIG. 4A). This behavior was never observed in the no-competition model; increasing E2 resulted in higher YFP expression for all explored parameter values (Fig. S1B). This result suggests that the most parsimonious model of promoter competition is sufficient to explain the data, and highlights the need to characterize synthetic parts over large dynamic ranges in order to be able to use them predictably for circuit construction.

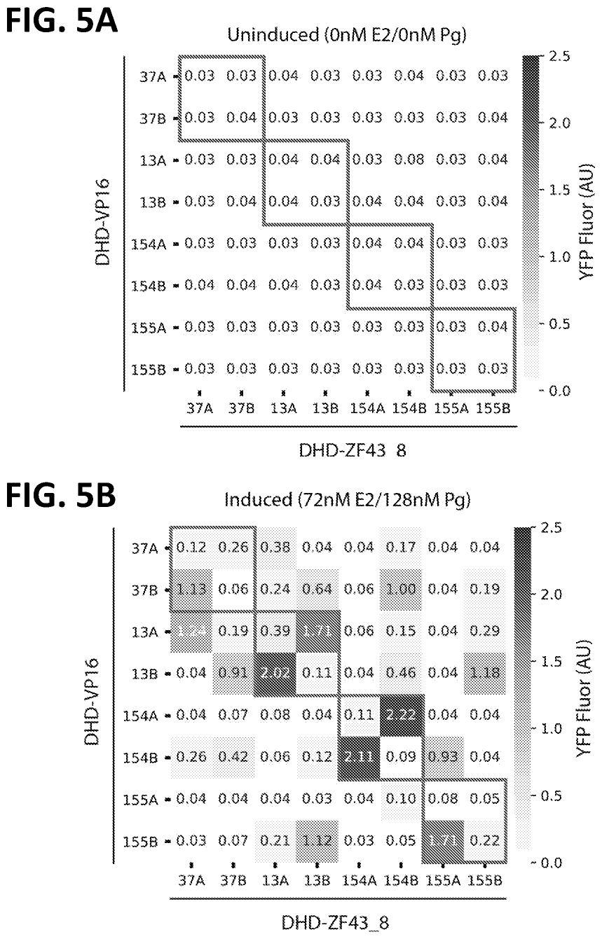

With this information at hand, the relative strengths of interactions between different DHD parts were determined, testing a matrix of on-target and off-target pairs. A subset of four on-target DHD pairs were selected (37A/B, 13A/B, 154A/B, and 155A/B) and strains were constructed for all possible 64 combinations of on- and off-target pairings in the split TF assay. YFP expression was then measured in the presence of saturating concentrations of both hormone inducers (FIG. 5B) and absence of both inducers (FIG. 5A). These two values were used to calculate the fold-change YFP expression upon induction of both DHD-VP16 and DHD-ZF43_8 (FIG. 1D). It was noticed that activation of the split TF was not symmetric for all DHD pairs. For both the 37 and 155 DHD on-target interactions, the split TF was only active when the DHD A monomer was fused to ZF43_8 and the B monomer was fused to VP16. The asymmetry could be a function of structure-function changes with fused proteins^{29,30}, and these observations helped inform downstream circuit design. In addition to the on-target interactions, it was observed that several interactions of varying strengths among off-target DHD pairs, of which only the 13B/37B interaction was previously identified via growth-based two-hybrid characterization.²⁶ The assay also revealed a range of binding activities for some DHDs to multiple off-target DHDs. For example, 154B-ZF43_8 interacted with 37A-VP16 weakly, 13B-VP16 moderately, and 37B-VP16 strongly. Nonetheless, the designed interaction partner always displayed the strongest split TF activation for a given DHD.

Given the full interaction matrix, it was hypothesized that a high-affinity, on-target interaction, which is referred to as the dominant negative (DN), might be able to inhibit and potentially reverse interactions between weaker DHD pairs, enabling new functionality in heterodimer-based circuits. To test this hypothesis, 154B was selected as a model DN because it exhibited a range of interaction strengths with other DHDs (FIG. 1D). A new circuit was built in which E2-induced pGAL1 drives expression of both 154B-VP16 and an off-target DHD (DHDoff): 155A-ZF43_8, 37B-ZF43_8 or 37A-ZF43_8 (FIG. 2A). The reconstituted split TF activates the p43_8 promoter to drive expression of a YFP-cODC degron fusion; the degron enables more rapid turnover of the circuit to more efficiently capture the displacement phenomenon.³¹ Concurrently, Pg-induced pZ3 drives expression of 154A, which acts as the high-affinity DN for 154B to potentially inhibit the split TF interaction. In a different permutation of the circuit designed to check for fusion effects, 154B was fused to ZF43_8 instead of VP16, and the various DHDoff were fused to VP16 (FIG. 2B). To test the ability of DN to compete with heterodimer formation, cells were simultaneously induced with a saturating dose of E2 and a range of Pg concentrations (FIG. 2C). YFP fluorescence was measured six hours later. Expression of the DN indeed reduced YFP expression by inhibiting the weaker-affinity, active TF through the formation of high-affinity, inactive complexes (FIG. 2D,E).

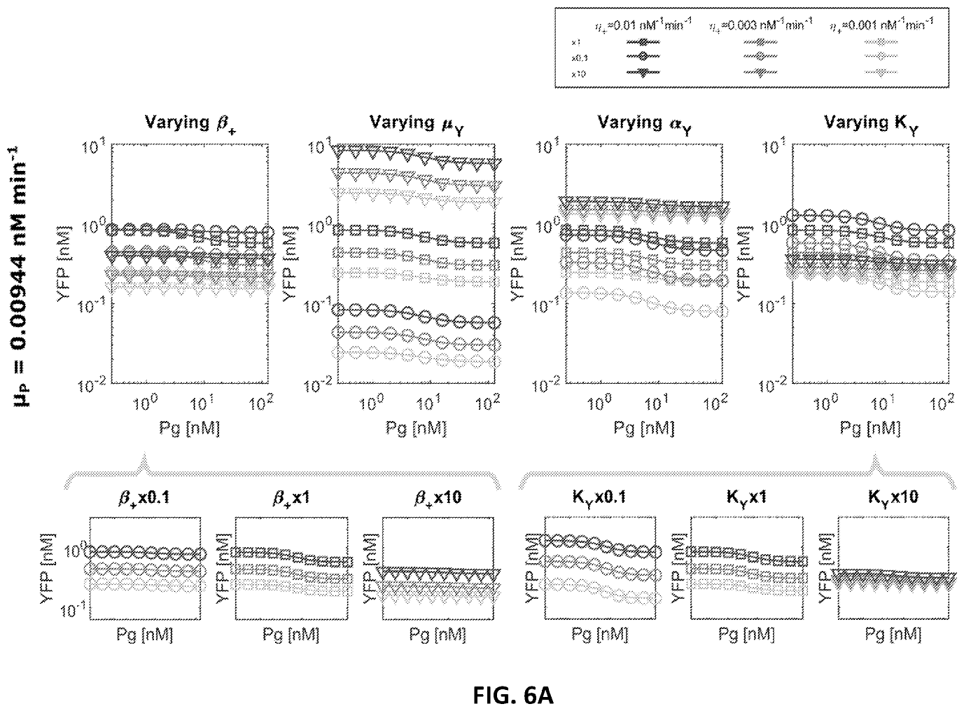

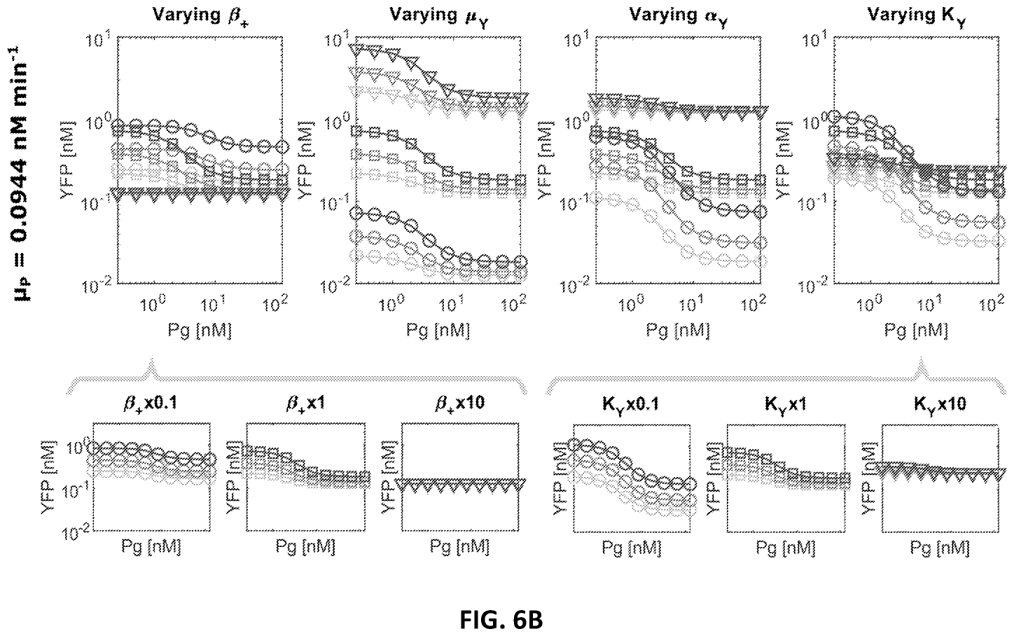

A higher concentration of the 154A DN was required to fully inhibit the split TF when 154B was fused to VP16 (FIG. 2D) than when 154B was fused to ZF43_8 (FIG. 2E). When the DN bound 154B-ZF43_8, YFP expression dropped off precipitously with small amounts of Pg (FIG. 2E). This difference cannot be explained simply by the promoter competition of inactive DBD parts (either free or bound to the DN part) as in the competition model proposed above, because the total concentration of the promoter binding (hence repressive) species in both circuit configurations is the same and determined by the E2 concentration (FIG. 2A-B). However, the model shows that varying the binding rates between on-target species (i.e. between DN and the sequestered part, either AD or DBD) can shift the dose response just as observed with the tested designs, suggesting that alternative fusion arrangements for 154B may alter its binding affinity to the DN (FIG. 6A-B; FIG. 2D-E). The experimental testing of DHD interactions in FIG. 1D supports this hypothesis; the same pair of DHDs could display different affinities depending on whether the monomer was fused to AD or DBD.

While this first assay tested the ability to form high-affinity complexes in the presence of weak-affinity binders, the application of the DN to dynamic signaling circuits requires the ability to displace existing complexes. Using the same circuits, whether the 154A DN would be able to competitively displace the weaker off-target DHD bound to 154B after the DHDoff and 154B pair reached binding equilibrium was tested. To do so, one circuit from each layout (FIGS. 2A and 2B) was exposed to a saturating dose of E2 for 4 hours to express both parts of the split TF, leaving ample time for the split TF to reach equilibrium and form active complexes.²⁷ After 4 hours of such E2 induction, cells were induced with a saturating dose of Pg to activate production of 154A DN and measured YFP fluorescence over another 4 hours (FIG. 2F). To enable comparison between the two circuit layouts, YFP fluorescence was normalized by subtracting the background fluorescence and dividing by the maximum fluorescence output of each circuit. A decrease in YFP fluorescence was observed for both circuits within an hour of DN induction (FIG. 2F). The rapid decay in transcriptional output suggests that the DN can inhibit the split TF even after the weaker-affinity DHDs have reached binding equilibrium. Moreover, the 154A DN deactivated the split TF faster for 154B-ZF43_8 than for 154B-VP16. This result is in agreement with the previous observation that 154A is a more potent DN when creating 154A: 154B-ZF43_8 complexes. These experiments demonstrate that dominant negative DHD interactions can be a powerful tool for reversible regulation of the activity of split proteins and outline several parameters for tuning the strength and dynamics of this inhibitory reaction.

To capitalize on the ability of the DHDs to dynamically assemble and interchange components, it was used a genetic circuit. Specifically, the interruption of the split TF through formation of a DN interaction was used used to build a modular negative feedback structure in a circuit simply by producing the DN part from the output of this circuit (FIG. 3A). To test this idea, a circuit was built in which the synthetic transcription factor GEM (induced by E2) activates the pGAL1 promoter to produce both parts of a split TF (154B-VP16 and 155A-ZF43_8 species). When the split TF heterodimer forms, it activates the p43_8 promoter to produce an intermediate reporter RFP, as well as the synthetic transcription factor Z3PM. Z3PM in turn activates the pZ3 promoter to drive expression of the dominant negative 154A, implementing negative feedback by binding to 154B-VP16 and inhibiting the formation of the split TF. Z3PM also binds to another copy of the pZ3 promoter to transcribe two copies of a YFP-cODC output. Z3PM and RFP are fused to a photosensitive degron (psd), which acts as a weak degron. With the cODC degron, the DN will have a similar turnover rate as the YFP to more closely approximate the output of the circuit.

At a given concentration of E2 and Pg, the system settles at a given steady-state. In the presence of feedback, increasing Pg disturbs the system from its steady-state. This increase should also increase DN expression, which can compensate for the Pg disturbance by inhibiting the split TF and decreasing the production rate of Z3PM and RFP. For comparison, two “No Feedback” controls that express the DN using two constitutive promoters (pC) with different strengths were constructed.

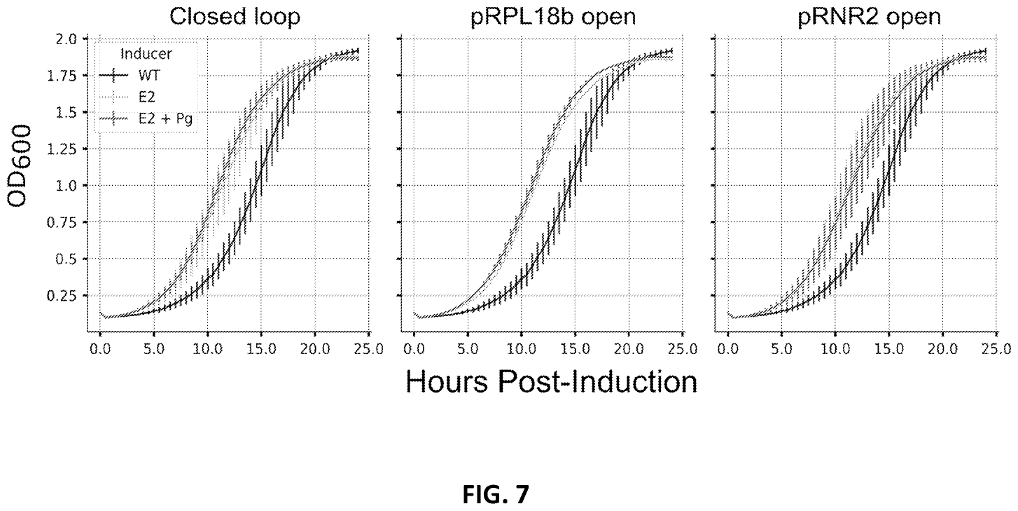

Whether the feedback circuit and its no-feedback control imposed a growth burden on yeast was tested. To do so, cells were induced with a saturating concentration of E2 and Pg, and compared the growth of cells harboring the circuit with the growth of the background stain over 24 hours (FIG. 7). There was no observable difference in growth at any inducer combination. Interestingly, cells harboring the circuits grew faster than the background strain, likely due to the prototrophy conferred by integrating the circuit components.

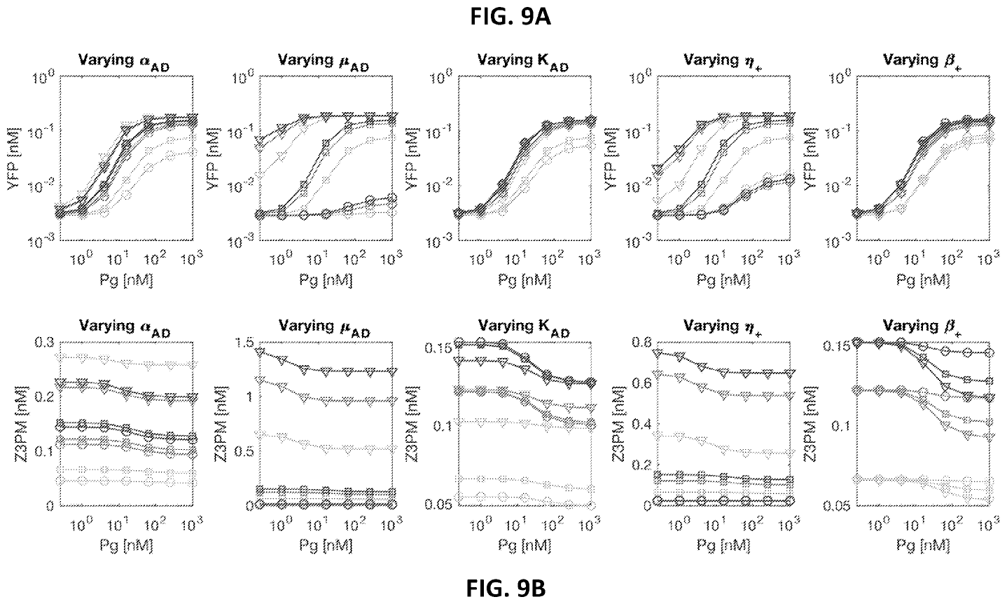

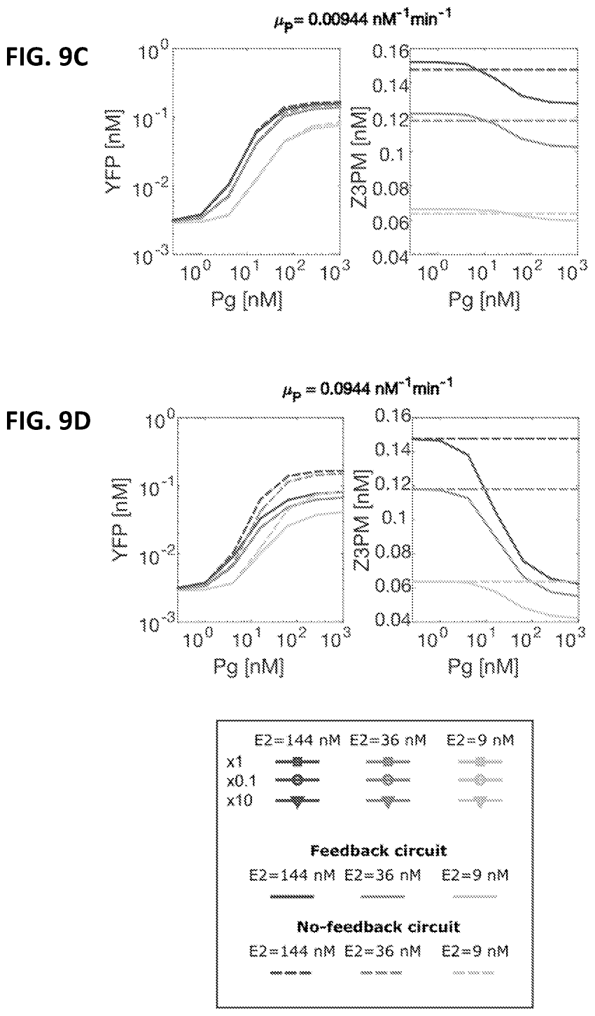

To assess the properties of the feedback circuit, the responses of the circuits with and without feedback as a function of Pg for a fixed concentration of E2 were compared (144 nM E2, FIG. 3B, FIG. 8). In both “No Feedback” circuits, the YFP output increases unabatedly as Pg increases. Additionally, the RFP remains constant, as the DN production is, by design, fixed and insensitive to Pg. By contrast, the feedback circuit increased production of the DN with increasing Pg. As expected, increasing DN inactivated more split TF, reducing activity of the p43_8 promoter, which was apparent as a decreasing RFP fluorescence (a proxy for decreasing Z3PM). The decreasing Z3PM concentration compensates for the higher Pg, thereby controlling the effect of this disturbance on the YFP output (FIG. 3B). Furthermore, different constitutive levels of expression of the DN in the different circuits were unable to recapitulate this behavior, highlighting the unique properties of the feedback circuit (FIG. 3B, FIG. 8). To test the general understanding of the strategy of the feedback circuit and its qualitative properties, a computational model was built that recapitulated the behavior of the experimental results using the same parameters used in the previous models (FIG. 9).

A feedback system would be less useful if its output cannot be tuned over some range by changing its input. To explore the ability to tune the output of the feedback circuit, cells were induced at several different E2 concentrations, while scanning the full range of Pg concentrations. The output fluorescence at steady-state was measured. The RFP and YFP fluorescence was normalized to the maximum fluorescence value observed (FIG. 3C) to enable comparison between the circuit variants (see FIG. 8 for non-normalized RFP and YFP outputs). In the presence of feedback, both RFP and YFP fluorescence display a clear dependence on E2. Increasing Pg decreased the response of the RFP/E2 relationship, whereas it increased the response of the YFP/E2 relationship (FIG. 3C). This suggests that E2 input can be used to specify the level of output in the circuit when feedback mitigates changes in Pg concentration. These results indicate that negative feedback implemented through competitive displacement is a powerful method for shaping the response of circuits in cells.

### Model Descriptions

### Promoter Occupancy Model

We want to explore if the competition for promoter binding between the split TF (i.e. the heterodimer, AD:DBD) and the free DBD part can explain the paradoxical behavior observed in FIG. 1C, where increasing DBD part expression (by increasing E2 concentration) reduces, instead of increasing, the output expression.

Let's consider that the output promoter can be in three forms: free (P), bound to the split TF—heterodimer AD:DBD—(P_{A}), or bound to the free DBD part (P_{D}). The total number of promoters, P_{T}=P+P_{D}+P_{A}, remains constant. Then, the transcription rate associated with the promoter can be expressed as

$${f}_{Y} = {μ}_{Y} ( {α}_{Y} \cdot \frac{P}{{P}_{T}} + \frac{{P}_{A}}{{P}_{T}} ) ,$$

where μ_{Y} is the transcription rate by the active promoter (i.e. bound to the split TF), and α_{Y}∈[0,1] is a factor rescaling the transcription rate to account for the basal activity of the free promoter. The free DBD part works as a repressor inhibiting transcription, such that Pp has zero transcription rate.

Assuming that the promoter occupancy is in quasi-equilibrium compared to the transcriptional step, and that the binding/unbinding rates of the heterodimer or the free DBD part to the promoter are the same, the following expression is obtained:

$$P = {P}_{T} - {P}_{A} - {P}_{D}$$

$${dP}_{D} / dt = {ω}_{+} P \cdot D - {ω}_{-} {P}_{D} = 0$$

$${dP}_{D} / dt = {ω}_{+} ( {P}_{T} - {P}_{A} - {P}_{D} ) \cdot D - {ω}_{-} {P}_{D} = 0$$

$$⇒ {P}_{D} = ( {P}_{T} - {P}_{A} ) \frac{{ω}_{+} D}{{ω}_{+} D + {ω}_{-}}$$

$${dP}_{A} / dt = {ω}_{+} P \cdot TF - {ω}_{-} {P}_{A} = 0$$

$${dP}_{A} / dt = {ω}_{+} ( {P}_{T} - {P}_{A} - ( {P}_{T} - {P}_{A} ) \frac{{ω}_{+} D}{{ω}_{+} D + {ω}_{-}} ) \cdot TF - {ω}_{-} {P}_{A} = 0$$

$$⇒ \frac{{P}_{A}}{{P}_{T}} = \frac{{ω}_{+} TF}{{ω}_{-} + {ω}_{+} TF + {ω}_{+} D}$$

$${f}_{Y} ( TF , D ) = {μ}_{Y} ( {α}_{Y} \cdot \frac{P}{{P}_{T}} + \frac{{P}_{A}}{{P}_{T}} ) = {μ}_{Y} \cdot \frac{{α}_{Y} {ω}_{-} + {ω}_{+} TF}{{ω}_{-} + {ω}_{+} TF + {ω}_{+} D} = {μ}_{Y} \cdot \frac{{α}_{Y} {K}_{Y} + TF}{{K}_{Y} + TF + D}$$

where

$${K}_{Y} = \frac{{ω}_{-}}{{ω}_{+}}$$

corresponds to the dissociation constant between the DBD (free, D, or where in the heterodimer complex AD:DBD, TF) to the promoter. Under the alternative model where the free DBD (D) competition for the promoter is negligible (i.e. assuming P_{T}=P+P_{A}), the contribution of D in the promoter is simply removed from the denominator of the function:

$${f}_{Y} ( TF ) = {μ}_{Y} ( {α}_{Y} \cdot \frac{P}{{P}_{T}} + \frac{{P}_{A}}{{P}_{T}} ) = {μ}_{Y} \cdot \frac{{α}_{Y} {K}_{Y} + TF}{{K}_{Y} + TF} .$$

Using the synthesis function and parameters reported in Gómez-Schiavon, Dods et al. (2020) for the individual parts synthesis rate (f_{P}(Z3PM_{Pg}, Z3PM), f_{E}(GEM_{E2}, GEM)) as the hormone concentrations vary, the heterodimer system shown in FIG. 1A is modeled as follows:

$$\frac{d}{dt} A = {f}_{P} ( Z 3 {PM}_{Pg} , Z 3 PM ) - γ A + {η}_{-} TF - {η}_{+} A \cdot D$$

$$\frac{d}{dt} D = {f}_{E} ( {GEM}_{E 2} , GEM ) - γ A + {η}_{-} TF - {η}_{+} A \cdot D$$

$$\frac{d}{dt} TF = - γ TF - {η}_{-} TF + {η}_{+} A \cdot D$$

$$\frac{d}{dt} Y = {f}_{Y} ( TF , D ) - γ Y$$

where A represents the concentration of the free AD part, D of the free DBD part, TF of the heterodimer of AD and DBD, and Y of the YFP reporter. Assuming that all molecules are lost by dilution (γ), and η₊, η₋ correspond to the binding and unbinding rates, respectively, of the heterodimer parts.

In FIG. 4, the qualitative behavior of this promoter competition model was evaluated for different parameter values. For all the cases explored, the promoter

$$competition model ( {f}_{Y} ( TF , D ) = {μ}_{Y} \frac{{α}_{Y} {K}_{Y} + TF}{{K}_{Y} + TF + D} )$$

displays the paradoxical effect observed in FIG. 1C: increasing the DBD part expression (D_{T}=D+TF) by increasing estradiol concentration (E2) reduces the YFP reporter expression (Y; FIG. 4A). On the other hand, using the alternative model where the competition by the free DBD part for the promoter is considered negligible

$$( no - competition model , {f}_{Y} ( TF ) = {μ}_{Y} \frac{{α}_{Y} {K}_{Y} + TF}{{K}_{Y} + TF} ) ,$$

increasing E2 resulted in higher

YFP expression for all explored parameter values (FIG. 4B). Therefore, the simple competition between the heterodimer—or active transcription factor, TF—and the free DBD part for the promoter occupancy is sufficient to explain the observed paradoxical effect. It was also observed that the inhibiting effect of higher estradiol (E2; i.e. the decrement of YFP expression as estradiol concentration increases) is more significant for lower progesterone concentrations (Pg; FIG. 4A). This behavior is also expected under the competition model: Lower progesterone results in lower AD part expression (A_{T}=A+TF), limiting the amount of heterodimer

$$( TF ; \frac{d}{dt} TF = 0 ⇔ TF = \frac{{η}_{+}}{{η}_{-} + γ} A \cdot D ) .$$

As increasing estradiol results in higher DBD part expression (D_{T}=D+TF), more free DBD part concentration, D, is expected relative to the concentration of the active transcription factor, TF. The free DBD part concentration competes for the promoter binding, increasing the effective repression.

### Dominant Negative Model

We now explore the addition of the dominant negative part (DN, N) to the model. This DN part can sequester either the AD part (A; FIG. 2A) or the DBD part (D; FIG. 2B). In the latter case, the DN: DBD (DD) complex can also compete for the YFP promoter, and it can be assumed this occurs with the same affinity as the free DBD part and the complex AD:DBD. Also, lacking the activation domain, the DN: DBD can work as a repressor just as the free DBD part in the competition model described above. Then, under the promoter competition model described above, it is easy to see that the YFP synthesis rate for any of these designs can be written as

$${f}_{Y} ( TF , {D}_{T} ) = {μ}_{Y} \frac{{α}_{Y} {K}_{Y} + TF}{{K}_{Y} + {D}_{T}} ,$$

where D_{T} is the total DBD part concentration, either in its free form (D), the split TF heterodimer (TF), or the dominant negative complex (DN: DBD, DD), if the DN part sequesters the DBD part (otherwise, DD=0).

Once again using the synthesis function and parameters reported in Gómez-Schiavon, Dods et al. (2020) for the individual parts synthesis rate (f_{P}(Z3PM_{Pg}, Z3PM), f_{E}(GEM_{E2}, GEM)) as the hormone concentrations vary, and the competition model described above

$$( {f}_{Y} ( TF , {D}_{T} ) = {μ}_{Y} \frac{{α}_{Y} {K}_{Y} + TF}{{K}_{Y} + {D}_{T}} ) ,$$

the dominant negative system shown in FIG. 2A is modeled as follows:

$$\frac{d}{dt} A = {f}_{E} ( {GEM}_{E 2} , GEM ) - γ A + {η}_{-} TF - {η}_{+} A \cdot D + {β}_{-} AN - {β}_{+} A \cdot N$$

$$\frac{d}{dt} D = {f}_{E} ( {GEM}_{E 2} , GEM ) - γ D + {η}_{-} TF - {η}_{+} A \cdot D$$

$$\frac{d}{dt} N = {f}_{P} ( Z 3 {PM}_{Pg} , Z 3 PM ) - γ N + {β}_{-} AN - {β}_{+} A \cdot N$$

$$\frac{d}{dt} TF = - γ TF - {η}_{-} TF + {η}_{+} A \cdot D$$

$$\frac{d}{dt} AN = - γ AN - {β}_{-} AN + {β}_{+} A \cdot N$$

$$\frac{d}{dt} Y = {f}_{Y} ( TF , {D}_{T} ) - γ Y$$

where A represents the concentration of the free AD part, D of the free DBD part, N of the free DN part, TF of the heterodimer split transcription factor, AN of the AD:DN complex, and Y of the YFP reporter. Assuming that all molecules are lost by dilution (γ), η₊, η₋ correspond to the binding and unbinding rates, respectively, of the AD, DBD parts, β₊, β₋ correspond to the binding and unbinding rates, respectively, of the AD, DN parts, and D_{T}=D+TF is the total DBD.

Similarly, the dominant negative system shown in FIG. 2B is modeled as follows:

$$\frac{d}{dt} A = {f}_{E} ( {GEM}_{E 2} , GEM ) - γ A + {η}_{-} TF - {η}_{+} A \cdot D$$

$$\frac{d}{dt} D = {f}_{E} ( {GEM}_{E 2} , GEM ) - γ D + {η}_{-} TF - {η}_{+} A \cdot D + {β}_{-} DD - {β}_{+} D \cdot N$$

$$\frac{d}{dt} N = {f}_{P} ( Z 3 {PM}_{Pg} , Z 3 PM ) - γ N + {β}_{-} DD - {β}_{+} D \cdot N$$

$$\frac{d}{dt} TF = - γ TF - {η}_{-} TF + {η}_{+} A \cdot D$$

$$\frac{d}{dt} DD = - γ DD - {β}_{-} DD + {β}_{+} D \cdot N$$

$$\frac{d}{dt} Y = {f}_{Y} ( TF , {D}_{T} ) - γ Y$$

where DD represents the concentration of the DN: DBD complex, β₊, β₋ correspond now to the binding and unbinding rates, respectively, of the DN, DBD parts, and D_{T}=D+DD+TF is the total DBD. All other definitions and assumptions are still as above

Comparing the model of both designs above, it can be observed that the total DBD concentration (D_{T}) is independent of the sequestration design chosen (DN part sequestering either the AD or DBD parts; in both systems, at steady state D_{T}=f_{E}(GEM_{E2}, GEM)/γ). Moreover, the effect of reducing either the amount of AD or DBD parts (e.g. through DN part sequestration) over the heterodimer (TF) steady state value is completely analogous, with

$$TF = \frac{{η}_{+}}{γ + {η}_{-}} A \cdot D .$$

Then, the repression effect and output of both sequestration designs is expected to be the same when AD and DBD, respectively, have the same affinity for the dominant negative in both systems, and are present in equivalent concentrations. Consequently, the differential behavior observed in FIG. 2A-B cannot be explained by the sequestration design alone.

We propose that the observed differential behavior between both sequestration designs (FIG. 2A-B) is a consequence of changes in the parts affinity as well as the individual parts expression levels. First, the affinity between heterodimer parts can clearly vary as their fusion part varies (either the activation domain, the DNA-binding domain, or none as in the dominant negative part), as suggested by the observed asymmetry in FIG. 1D. Second, the expression of the DN part can be expected to be more efficient than the AD and DBD parts as it lacks the corresponding fusions. The model suggests that the qualitative behavior of this system is highly sensitive to the relative concentration of the dominant negative part (N) compared to the sequestered part (either AD, A, or DBD, D; FIG. 6). For instance, using the maximum synthesis rate (μ_{P}) for the pZ3 promoter (under the regulation of Z3PM) estimated in Gómez-Schiavon, Dods et al. (2020), the proposed model fails to capture the qualitative behavior of the experimental system (FIG. 6A). The effect of increasing the value of μ_{P} on the qualitative behavior of the model was explored (FIG. 6B). Higher expression of the DN relative to what is expected under the same conditions for the AD or DBD parts (considering the parameters reported in Gómez-Schiavon, Dods et al., 2020 were estimated under analogous conditions for f_{P}(Z3PM_{Pg}, Z3PM), f_{E}(GEM_{E2}, GEM)) is enough for the model to recapitulate the qualitative behavior observed in FIG. 2D-E for a wide range of parameter values (e.g. μ_{P}=0.00944 nM min⁻¹, instead of μ_{P}=0.00944 nM min⁻¹ as reported in Gómez-Schiavon, Dods et al.). Moreover, the model predicts that changes in the affinity of the DN part for the sequestered part (either AD or DBD) can have a similar effect as the variation observed between FIG. 2D and FIG. 2E, supporting the hypothesis that the effect of the fusion is responsible for the differential behavior of both sequestering designs.

### Feedback Circuit Model

Finally, the behavior of the negative feedback circuit was explored (FIG. 3A) with computational modeling, based on the promoter competition model described above.

Once again using the synthesis function and parameters reported in Gómez-Schiavon, Dods et al. (2020) for the individual parts synthesis rate (f_{P}(Z3PM_{Pg}, Z3PM), f_{E}(GEM_{E2}, GEM)) as the hormone concentrations vary, and the competition model for the Z3PM synthesis under the heterodimer regulation

$$( {f}_{AD} ( TF , {D}_{T} ) = {μ}_{AD} \frac{{α}_{AD} {K}_{AD} + TF}{{K}_{AD} + {D}_{T}} ,$$

analogous to the f_{Y}(TF, D_{T}) described above), the feedback system shown in FIG. 3A is modeled as follows:

$$\frac{d}{dt} A = {f}_{E} ( {GEM}_{E 2} , GEM ) - γ A + {η}_{-} TF - {η}_{+} A \cdot D + {β}_{-} AN - {β}_{+} A \cdot N + {γ}_{N} \cdot AN$$

$$\frac{d}{dt} D = {f}_{E} ( {GEM}_{E 2} , GEM ) - γ D + {η}_{-} TF - {η}_{+} A \cdot D$$

$$\frac{d}{dt} N = {f}_{P} ( Z 3 {PM}_{Pg} , Z ) - ( γ + {γ}_{N} ) N + {β}_{-} AN - {β}_{+} A \cdot N$$

$$\frac{d}{dt} TF = - γ TF - {η}_{-} TF + {η}_{+} A \cdot D$$

$$\frac{d}{dt} AN = - ( γ + {γ}_{N} ) AN - {β}_{-} AN + {β}_{+} A \cdot N$$

$$\frac{d}{dt} Z = {f}_{AD} ( TF , {D}_{T} ) - ( γ + {γ}_{Z} ) Z$$

$$\frac{d}{dt} Y = {f}_{P} ( Z 3 {PM}_{Pg} , Z ) - ( γ + {γ}_{Y} ) Y$$

where A represents the concentration of the free AD part, D of the free DBD part, N of the free DN part, TF of the AD:DBD heterodimer, AN of the AD:DN complex, Z of the Z3PM transcription factor, and Y of the YFP reporter. Again, all molecules are assumed to be lost by dilution (γ), and the addition of degron tags to some parts is taken in account by including an extra degradation rate: γ_{Y} accounts for the degron added to YFP, γ_{Z} for Z3PM, and γ_{N} for the DN part. As in the models above, η₊, η₋ correspond to the binding and unbinding rates, respectively, of the AD,DBD parts; and, as the DN part sequesters the AD part, β₊, β₋ correspond to the binding and unbinding rates, respectively, of the AD,DN parts, and D_{T}=D+TF is the total DBD.

We explored the qualitative behavior of this feedback circuit model by tracking the YFP and Z3PM (analogous to RFP in the experimental settings) expression level with different parameter values (FIG. 9). The model suggests that the behavior of the system can be highly sensitive to the Z3PM maximum transcription rate (μ_{AD}) and the binding affinity (η₊) of activation domain to DNA binding domain, while the binding affinity (β₊) of the activation domain to the dominant negative has little effect on the system's qualitative response. Moreover, for low values μ_{AD} and η₊, the model recapitulates the qualitative behavior observed in the experimental setting (FIG. 3B).

We then tested if the model can capture the differences between the feedback and the no-feedback circuits observed in the experiment data. The no-feedback model is similar to the feedback model except that the synthesis of the DN part (N) is assumed constitutive, with a constant synthesis rate μ₀:

$$\frac{d}{dt} N = {μ}_{O} - ( γ + {γ}_{D} ) N + β\_AN - {β}_{+} A \cdot N$$

Using the same nominal parameter values as in FIG. 9A-B, the no-feedback circuit dose response to changes in progesterone concentration (Pg) was simulated for several concentrations of estradiol (E2), and compared the behavior of the feedback and the no-feedback circuits (FIG. 9C). As expected, the no-feedback circuit showed constant Z3PM expression regardless of Pg concentration, and the feedback circuit showed decreased Z3PM expression as Pg concentration increased. Nevertheless, using the parameter values in Gómez-Schiavon, Dods et al. (2020), the YFP expression level in the no-feedback circuit was only slightly higher than the feedback circuit. This difference is significantly smaller than what was observed in the experimental system. Inspired by the observation in FIG. 6, where the relative expression of the DN part was shown to qualitatively affect the dose response of the sequestration system, the effect of varying the DN part maximum synthesis rate (μ_{P}) on the feedback circuit dose response was explored. Interestingly, a higher DN part synthesis rate also allowed this model to recapitulate the qualitative behavior observed in the experiments (FIG. 3B). Using μ_{P}=0.0944 nM min⁻¹ (instead of μ_{P}=0.00944 nM min⁻¹ as reported in Gómez-Schiavon, Dods et al.; FIG. 9D), the Z3PM level of the feedback system is now further suppressed as the Pg concentration increases, resulting in larger differences of the YFP level between the feedback and the no-feedback system. Once again, higher μ_{P} values would be expected in the experimental system as the DN part is significantly smaller (and then potentially easier to express) than the AD and DBD parts, and both the dominant negative and feedback models suggest this is the case in the experimental system.

### Discussion

In this work, it has been demonstrated that de novo designed dimerizing systems can be used to build circuits in living cells. Specifically, on-target and off-target interactions of these systems were used to implement controllable reversibility, inducing and interrupting the activity of a TF at will. Once this basic module was established, and due to its modularity, networking it in a feedback circuit was possible. Aided by a titratable split TF system that allowed us to measure a range of DHD interaction strengths and identify several low-affinity off-target interactions, as well as a simple model to guide the understanding of the non-intuitive properties of the DHD parts, informed design choices could be made as more complex circuits were designed. This work showcases the importance of quantitatively characterizing titrable responses of designed proteins in order to facilitate informed design of complex circuits.

Previous work has leveraged autoinhibitory coiled-coils as a method for rapid sensing of proteolytic events.^{6.8} In this arrangement, weakly interacting coiled-coils are covalently fused with a linker containing a protease cleavage site. Proteolytic cleavage of the linker releases the weakly interacting coiled-coil and enables a higher affinity coiled-coil to interact with its binding partner and either activate or deactivate a split protein. While this system is a powerful method for coupling post-translational sensing to digital logic, the irreversible nature of proteolytic cleavage presents a major obstacle to using this motif in dynamic signaling circuits.

Others have demonstrated that competitive displacement can be used to construct dynamic signaling circuits. Bashor et al. constructed accelerator and delay circuits by inducibly expressing signaling modulators fused to high-affinity binders to outcompete a low-affinity binder, thus modifying the dynamics of the yeast mating pathway.¹⁶ The endogenous regulators used in this work are active even in the absence of the additional heterodimerization components, making it difficult to separate the effect of inducible expression of the regulators from the effect of the variable affinity heterodimerization domains. By contrast, the split TF system used in the work exhibits no activity in the absence of additional heterodimerization domains. Therefore, the DN negative feedback circuit is a more minimal demonstration that competitive displacement can be used to regulate dynamic behavior in a synthetic system.

The DN motif described in this work could be applied to control the activity of other split proteins beyond the split TF. Of note, multiple split Cas9 variants have been constructed which rely on chemically induced dimerization to rescue nuclease activity.^{32,33} DHDs could substitute for chemical dimerization domains to activate split Cas9, and competitive displacement could enable reversible control of gene editing. Furthermore, multiple therapeutically-relevant split kinases and phosphatases have been developed.^{34,35} With multiple sensors and orthogonal DNs, post-translational circuits could be developed to dynamically control phosphorylation and dephosphorylation events in a cell via competitive displacement.³⁶ Lastly, the modularity of the DN motif enables its use in other circuit topologies beyond negative feedback, such as incoherent feedforward loops, which require at least one inhibitory reaction.³⁷ Competitive displacement is a powerful tool for controlling split protein signaling and has many potential applications in building genetic and protein circuits for therapeutic and biotechnological applications.

### REFERENCES

1. Klemm, J. D., Schreiber, S. L. & Crabtree, G. R. DIMERIZATION AS A REGULATORY MECHANISM IN SIGNAL TRANSDUCTION. Annual Review of Immunology vol. 16 569-592 (1998).
2. Jones, S. An overview of the basic helix-loop-helix proteins. Genome Biol. 5, 1-6 (2004).
3. Benezra, R., Davis, R. L., Lockshon, D., Turner, D. L. & Weintraub, H. The protein Id: A negative regulator of helix-loop-helix DNA binding proteins. Cell vol. 61 49-59 (1990).
4. Sun, X. H., Copeland, N. G., Jenkins, N. A. & Baltimore, D. Id proteins Id1 and Id2 selectively inhibit DNA binding by one class of helix-loop-helix proteins. Mol. Cell. Biol. 11, 5603-5611 (1991).
5. Lebar, T., Lainšček, D., Merljak, E., Aupič, J. & Jerala, R. A tunable orthogonal coiled-coil interaction toolbox for engineering mammalian cells. Nat. Chem. Biol. 16, 513-519 (2020).
6. Shekhawat, S. S., Porter, J. R., Sriprasad, A. & Ghosh, I. An autoinhibited coiled-coil design strategy for split-protein protease sensors. J. Am. Chem. Soc. 131, 15284-15290 (2009).
7. Gao, X. J., Chong, L. S., Kim, M. S. & Elowitz, M. B. Programmable protein circuits in living cells. Science 361, 1252-1258 (2018).
8. Fink, T. et al. Design of fast proteolysis-based signaling and logic circuits in mammalian cells. Nat. Chem. Biol. 15, 115-122 (2019).
9. Lee, M. J. et al. Engineered synthetic scaffolds for organizing proteins within the bacterial cytoplasm. Nat. Chem. Biol. 14, 142-147 (2017).
10. Lee, M. J. et al. De novo targeting to the cytoplasmic and luminal side of bacterial microcompartments. Nat. Commun. 9, 1-11 (2018).
11. Dueber, J. E., Yeh, B. J., Chak, K. & Lim, W. A. Reprogramming control of an allosteric signaling switch through modular recombination. Science 301, 1904-1908 (2003).
12. Dueber, J. E., Mirsky, E. A. & Lim, W. A. Engineering synthetic signaling proteins with ultrasensitive input/output control. Nat. Biotechnol. 25, 660-662 (2007).
13. Buchler, N. E. & Cross, F. R. Protein sequestration generates a flexible ultrasensitive response in a genetic network. Molecular Systems Biology vol. 5 272 (2009).
14. Khalil, A. S. et al. A synthetic biology framework for programming eukaryotic transcription functions. Cell 150, 647-658 (2012).
15. Bashor, C. J. et al. Complex signal processing in synthetic gene circuits using cooperative regulatory assemblies. Science 364, 593-597 (2019).
16. Bashor, C. J., Helman, N. C., Yan, S. & Lim, W. A. Using engineered scaffold interactions to reshape MAP kinase pathway signaling dynamics. Science 319, 1539-1543 (2008).
17. Whitaker, W. R., Davis, S. A., Arkin, A. P. & Dueber, J. E. Engineering robust control of two-component system phosphotransfer using modular scaffolds. Proc. Natl. Acad. Sci. U.S.A 109, 18090-18095 (2012).
18. Ryu, J. & Park, S.-H. Simple synthetic protein scaffolds can create adjustable artificial MAPK circuits in yeast and mammalian cells. Sci. Signal. 8, ra66 (2015).
19. Groves, B., Khakhar, A., Nadel, C. M., Gardner, R. G. & Seelig, G. Rewiring MAP kinases in Saccharomyces cerevisiae to regulate novel targets through ubiquitination. Elife 5, (2016).
20. Dueber, J. E. et al. Synthetic protein scaffolds provide modular control over metabolic flux. Nat. Biotechnol. 27, 753-759 (2009).
21. Thomik, T., Wittig, I., Choe, J.-Y., Boles, E. & Oreb, M. An artificial transport metabolon facilitates improved substrate utilization in yeast. Nat. Chem. Biol. 13, 1158-1163 (2017).
22. Klaus, M., D'Souza, A. D., Nivina, A., Khosla, C. & Grininger, M. Engineering of Chimeric Polyketide Synthases Using SYNZIP Docking Domains. ACS Chem. Biol. 14, 426-433 (2019).
23. Cho, J. H., Collins, J. J. & Wong, W. W. Universal Chimeric Antigen Receptors for Multiplexed and Logical Control of T Cell Responses. Cell 173, 1426-1438.e11 (2018).
24. Chen, Z. et al. De novo design of protein logic gates. Science 368, 78-84 (2020).
25. Thompson, K. E., Bashor, C. J., Lim, W. A. & Keating, A. E. SYNZIP protein interaction toolbox: in vitro and in vivo specifications of heterospecific coiled-coil interaction domains. ACS Synth. Biol. 1, 118-129 (2012).
26. Chen, Z. et al. Programmable design of orthogonal protein heterodimers. Nature 565, 106-111 (2019).
27. Aranda-Díaz, A., Mace, K., Zuleta, I., Harrigan, P. & El-Samad, H. Robust Synthetic Circuits for Two-Dimensional Control of Gene Expression in Yeast. ACS Synth. Biol. 6, 545-554 (2017).
28. Gómez-Schiavon, M., Dods, G., El-Samad, H. & Ng, A. H. Multidimensional Characterization of Parts Enhances Modeling Accuracy in Genetic Circuits. ACS Synth. Biol. 9, 2917-2926 (2020).
29. Saiz-Baggetto, S., Méndez, E., Quilis, I., Carlos Igual, J. & Carmen Bañó, M. Chimeric proteins tagged with specific 3×HA cassettes may present instability and functional problems. PLOS One 12, e0183067 (2017).
30. Lee, S., Lim, W. A. & Thorn, K. S. Improved Blue, Green, and Red Fluorescent Protein Tagging Vectors for S. cerevisiae. PLOS One 8, e67902 (2013).
31. Ng, A. H. et al. Modular and tunable biological feedback control using a de novo protein switch. Nature 572, 265-269 (2019).
32. Wright, A. V. et al. Rational design of a split-Cas9 enzyme complex. Proc. Natl. Acad. Sci. U.S.A 112, 2984-2989 (2015).
33. Nguyen, D. P. et al. Ligand-binding domains of nuclear receptors facilitate tight control of split CRISPR activity. Nat. Commun. 7, 12009 (2016).
34. Camacho-Soto, K., Castillo-Montoya, J., Tye, B., Ogunleye, L. O. & Ghosh, I. Small molecule gated split-tyrosine phosphatases and orthogonal split-tyrosine kinases. J. Am. Chem. Soc. 136, 17078-17086 (2014).
35. Diaz, J. E. et al. A Split-Abl Kinase for Direct Activation in Cells. Cell Chem Biol 24, 1250-1258.e4 (2017).
36. Mishra, D. et al. An engineered protein-phosphorylation toggle network with implications for endogenous network discovery. Science 373, (2021).
37. Alon, U. Network motifs: theory and experimental approaches. Nat. Rev. Genet. 8, 450-461 (2007).
38. Lee, M. E., DeLoache, W. C., Cervantes, B. & Dueber, J. E. A Highly Characterized Yeast Toolkit for Modular, Multipart Assembly. ACS Synth. Biol. 4, 975-986 (2015).

We claim:

1. A cell comprising a molecular switch comprising:
(a) a first polypeptide comprising a first part of a split protein and a first monomer of a designed heterodimer;
(b) a second polypeptide comprising a second part of a split protein and a second monomer of a designed heterodimer; and
(c) a third polypeptide comprising a third monomer of a designed heterodimer;
wherein either:
(i): (a) and (b) bind to each other via a relatively low affinity interaction between their monomers to produce a reconstituted split protein that has an activity is not provided by either (a) or (b) alone; and
(c) binds to (a) or (b) via a relatively high affinity between their monomers, thereby inactivating the reconstituted split protein; or
(ii): (a) and (c) bind to each other via a relatively low affinity interaction between their monomers; and
(b) binds to (a) via a relatively high affinity between their monomers to produce a reconstituted split protein that has an activity is not provided by either (a) or (b) alone.

2. A cell comprising a feedback circuit comprising:
(a) a first polypeptide comprising a first part of a split protein and a monomer of a designed heterodimer;
(b) a second polypeptide comprising a second part of a split protein and a monomer of a designed heterodimer; and
(c) a third polypeptide comprising a monomer of a designed heterodimer, not containing the first or second parts of the split protein,
wherein:
(a) and (b) bind to each other via an interaction between their monomers to produce a reconstituted split protein that has an activity is not provided by either (a) or (b) alone; and
(c) binds to (a) or (b) via an interaction between their monomers, thereby inactivating the reconstituted split protein; and
expression of (c) is regulated by the activity of the reconstituted split protein.

3. A cell comprising a feedforward circuit comprising:
(a) a first polypeptide comprising a first part of a split protein and a monomer of a designed heterodimer;
(b) a second polypeptide comprising a second part of a split protein and a monomer of a designed heterodimer;
(c) a third polypeptide comprising a monomer of a designed heterodimer, not linked to the first or second parts of the split protein, and
(d) an actuating protein that, in response to an external stimulus, independently activates expression of (c) and at least one of (a) and (b);
wherein:
(a) and (b) bind to each other via an interaction between their monomers to produce a reconstituted split protein that has an activity is not provided by either (a) or (b) alone; and
(c) binds to (a) or (b) via an interaction between their monomers, thereby inactivating the reconstituted split protein

4. The cell of claim 2 or 3, wherein
(a) and (b) bind to each other via a relatively low affinity interaction; and
(c) binds to (a) or (b) via a relatively high affinity interaction.

5. The cell of claim 4, wherein the affinity between (a) and (b) is at least 10× the affinity between (c) and (a) or (b).

6. The cell of any of claims 2-5, wherein monomer of (c) is the same the same as the monomer of (a) or (b).

7. The cell of claim 1, 2, 4 or 5, wherein expression of (a) and/or (b) are activated by a external stimulus.

8. The cell of any of claims 3-7, wherein the cell further comprises:
(e) a controller protein, wherein the controller protein that regulates the interaction between the (c) and (a) or (b).

9. The cell of any prior claim, wherein the third polypeptide comprises a degron.

10. The cell of any prior claim, wherein the monomers are selected from SEQ ID NOS: 1-298.

11. The cell of any prior claim, wherein the reconstituted split protein is transcription factor, enzyme, kinase or chimeric antigen receptor.

12. The cell of prior claim, wherein the split protein is a split transcription factor, the cell comprises an expression cassette comprising a promoter and a coding sequence that are operably linked, and transcription of the coding sequence is activated by the split transcription factor.

13. The cell of claim 12, wherein the coding sequence encodes a CAR, cytokine or enzyme.

14. The cell of any prior claim, wherein the cell is an immune cell.

15. The cell of claim 14, wherein the cell is a T cell, Natural Killer cell or macrophage.

16. The cell of claim 15, wherein the cell is a stem cell.

17. A cell comprising nucleic acid encoding the molecular switch, feedback circuit or feedforward circuit of any of claims 1-16.

18. A method comprising:
exposing a cell of any prior claim to the external stimulus, thereby actuating the switch, feedback control or feedforward control.

19. The method of claim 18, wherein the method is done in vivo, ex vivo, or in vitro.


SEQUENCE LISTING (299 sequences)

SEQ ID NO: 1 - protein, 64 aa, synthetic construct
  REGION 1..64: note = Synthetic peptide
GSPKEEARELIRKQKELIKEQKKLIKEAKQKSDSRDAERIWKRSREINRESKKINKRIKELIKS

SEQ ID NO: 2 - protein, 62 aa, synthetic construct
  REGION 1..62: note = Synthetic peptide
PKKEAEELAEESEELHDRSEKLHERAEQSSNSEEARKILEDIERISERIEEISDRIERLLRS

SEQ ID NO: 3 - protein, 77 aa, synthetic construct
  REGION 1..77: note = Synthetic peptide
GTKEDILERQRKIIERAQEIHRRQQEILEELERIIRKPGSSEEAMKRMLKLLEESLRLLKELLELSEESAQLLYEQR

SEQ ID NO: 4 - protein, 83 aa, synthetic construct
  REGION 1..83: note = Synthetic peptide
GTEKRLLEEAERAHREQKEIIKKAQELHRRLEEIVRQSGSSEEAKKEAKKILEEIRELSKRSLELLREILYLSQEQKGSLVPR

SEQ ID NO: 5 - protein, 76 aa, synthetic construct
  REGION 1..76: note = Synthetic peptide
TKEDILERQRKIIERAQEIIRRQQEILEELERIIRKPGSSEEAMKRMLKLLEESLRLLKELLELLEESAQLLYEQR

SEQ ID NO: 6 - protein, 78 aa, synthetic construct
  REGION 1..78: note = Synthetic peptide
GSTEKRLLEEAERAHREAKEIIKKAQELHRRLEEIVRQSGSSEEAKKEAKKILEEIRELSKRLLELLREILYLSQEQK

SEQ ID NO: 7 - protein, 76 aa, synthetic construct
  REGION 1..76: note = Synthetic peptide
TKEDILERARKIIERAQEIHRRQQEILEELERIIRKPGSSEEAMKRMLKLLEESLRLLKELLELSEELAQLLYEQR

SEQ ID NO: 8 - protein, 78 aa, synthetic construct
  REGION 1..78: note = Synthetic peptide
GSTEKRLLEEAERAIREQKEIIKKAQELHRRLEEIVRQSGSSEEAKKEAKKILEEIRELSKRSLELLREILYLLQEQK

SEQ ID NO: 9 - protein, 35 aa, synthetic construct
  REGION 1..35: note = Synthetic peptide
TKEDILERQRKIIERAQEIHRRQQEILEELEYIIR

SEQ ID NO: 10 - protein, 121 aa, synthetic construct
  REGION 1..121: note = Synthetic peptide
MSEEAMKRMLKLLEESLRLLKELLELSEESAQLLYEQRKANNGSETEKRLLEEAERAHREQKEIIKKAQELHRRLEEIVRQSGSSEEAKKEAKKILEEIRELSKRSLELLREILYLSQEQK

SEQ ID NO: 11 - protein, 77 aa, synthetic construct
  REGION 1..77: note = Synthetic peptide
MTKEDILERQRKIIERAQEIHRRQQEILKEQEKIIRKPGSSEEAMKRSLKLIEESLRLLKELLELSEESAQLLYEQR

SEQ ID NO: 12 - protein, 77 aa, synthetic construct
  REGION 1..77: note = Synthetic peptide
GTEKRLLEEAERAHREQKEIIKKAQELHKELTKIHQQSGSSEEAKKRALKISQEIRELSKRSLELLREILYLSQEQK

SEQ ID NO: 13 - protein, 76 aa, synthetic construct
  REGION 1..76: note = Synthetic peptide
TKEDILERQRKIIERAQEIHRRQQEILKRSEEIIRKPGSSEEALETLRELQEESLRLLKELLELSEESAQLLYEQR

SEQ ID NO: 14 - protein, 78 aa, synthetic construct
  REGION 1..78: note = Synthetic peptide
GSTEKRLLEEAERAHREQKEIIKKAQELHRRTEEIIRQSGSSEEAKDELRRIQEEIRELSKRSLELLREILYLSQEQK

SEQ ID NO: 15 - protein, 36 aa, synthetic construct
  REGION 1..36: note = Synthetic peptide
TTKRYLEEAERAHREQKEIIKKAQELHRRLEEIVRQ

SEQ ID NO: 16 - protein, 116 aa, synthetic construct
  REGION 1..116: note = Synthetic peptide
GSSEEAKKEAKKILEEIRELSKRSLELLREILYLSQQVNDVDEKALERQRKIIERAQEIHRRQQEILEELERIIRKPGSSEEAMKRMLKLLEESLRLLKELLELSEESAQLLYEAR

SEQ ID NO: 17 - protein, 35 aa, synthetic construct
  REGION 1..35: note = Synthetic peptide
EAMKRMLKLLEESLRLLKELLELSEESAQLLYEAR

SEQ ID NO: 18 - protein, 116 aa, synthetic construct
  REGION 1..116: note = Synthetic peptide
TTKRYLEEAERAHREQKEIIKKAQELHRRLEEIVRQSGSSEEAKKEAKKILEEIRELSKRSLELLREILYLSQQVNDVDEKALERQRKIIERAQEIHRRQQEILEELERIIRKPGS

SEQ ID NO: 19 - protein, 77 aa, synthetic construct
  REGION 1..77: note = Synthetic peptide
TREELLRENIELAKEHIEIMREILELLQKMEELLEKARGADEDVAKTIKELLRRLKEIIERNQRIAKEHEYIARERS

SEQ ID NO: 20 - protein, 78 aa, synthetic construct
  REGION 1..78: note = Synthetic peptide
GTERKLLERSRRLQEESKRLLDEMAEIMRRIKKLLKKARGADEKVLDELRKIIERIRELLDRSRKIHERSEEIAYKEE

SEQ ID NO: 21 - protein, 76 aa, synthetic construct
  REGION 1..76: note = Synthetic peptide
GDRQELIRRNIELLKEHIKILEEISQLIEELSELLDKSSSEEVVKRYKKILERYKQLLRKSQEIHKESSEIAKKES

SEQ ID NO: 22 - protein, 82 aa, synthetic construct
  REGION 1..82: note = Synthetic peptide
GDEQKLIERSQRMQKESLELLKEIIKILDTIEKLLDKPDSEELLDTIKKLHDTLKKIHDRNKKLLKEHEEILRQRSGSLVPR

SEQ ID NO: 23 - protein, 73 aa, synthetic construct
  REGION 1..73: note = Synthetic peptide
DKEEEYKRLLDEIKEILKESKEVLKDSKRVLEDIKRKVPDDDLVKLLEKHVRLLEEHVKLLEQLIREAEKSSK

SEQ ID NO: 24 - protein, 72 aa, synthetic construct
  REGION 1..72: note = Synthetic peptide
QGSSAEELLKKIKESEKKIRDSLRKIKEIIKKSRKEGVDDKQLDLIRKVVESHRDLLRLHRDLLRLLREETS

SEQ ID NO: 25 - protein, 77 aa, synthetic construct
  REGION 1..77: note = Synthetic peptide
DIDESIKEVEKLLEEVEQSLQKLDDSLKKLLEKVNQDPDVDDSVRKIVKRHVEILKRHEEVLKRLIEVVKEHTKTVK

SEQ ID NO: 26 - protein, 72 aa, synthetic construct
  REGION 1..72: note = Synthetic peptide
GSDREEVHKEIVKLIREIIKIHKKILKIHEKIKNGEIDPSEILKLSEEIKKLTDTIIKIIEDLEQLTRDLRR

SEQ ID NO: 27 - protein, 71 aa, synthetic construct
  REGION 1..71: note = Synthetic peptide
DRKEIVKRHQKVVELLKESSKLLRESSKLLQRLLDKTGDENLQKAVDDQDKAIKRQETAIRKSQEASKKLD

SEQ ID NO: 28 - protein, 77 aa, synthetic construct
  REGION 1..77: note = Synthetic peptide
DNSEEIKKVAKTSREVAEYSERVAKENDKVVKTLEEGKIDESELLRLLEESIKIFDTALKLHEEAYKLHQDLVRKVS

SEQ ID NO: 29 - protein, 72 aa, synthetic construct
  REGION 1..72: note = Synthetic peptide
DESEAASVAIESVQILVESVKLLEESVRILLDAVKKNGVEDLLRVAQRWEKLVDEWLKVVKRWLDNVRDIQR

SEQ ID NO: 30 - protein, 76 aa, synthetic construct
  REGION 1..76: note = Synthetic peptide
GSDKAEEVEKSVRKIEESIKKIRKSIKKAEDAVQLLKEGKIDAKDFLRIVREDLEVVKEDVEIVKEDVENVREFSS

SEQ ID NO: 31 - protein, 69 aa, synthetic construct
  REGION 1..69: note = Synthetic peptide
SDKEVSDKLLKASKKLLKVSEELLEVVRRLLKALKDDELIKKIADLLRKIIDKDKKFIRTSEEIVKESR

SEQ ID NO: 32 - protein, 78 aa, synthetic construct
  REGION 1..78: note = Synthetic peptide
GSDLKEVLKTVEEAVKEIIKSSEELLQISRKILEISRVGVDEHEYISAIREYLKALEKHIQILKKFIEILKELIRAVS

SEQ ID NO: 33 - protein, 81 aa, synthetic construct
  REGION 1..81: note = Synthetic peptide
SKEEIDKIVKKHKKKIEEHKKKVDELKKLVEEHDKRVSQDKDDKVKKLSEEVKKIIKRLEEVSKRLEEVSKKLLKVISDKR

SEQ ID NO: 34 - protein, 78 aa, synthetic construct
  REGION 1..78: note = Synthetic peptide
GSNDEELKKILETLDRILKKLDKILTRLIEVLKKSEDPNLDDKDYTELVKQFIELIKKYEEVVKEYEEVVRQLIRLFS

SEQ ID NO: 35 - protein, 81 aa, synthetic construct
  REGION 1..81: note = Synthetic peptide
SKEEIDKIVKKHKKKIEELKKLVDELKKLVEEHDKRVSQDKDDKVKKLSEEVKKIIKRVEEVAKRLEEVSKKLLKVISDKR

SEQ ID NO: 36 - protein, 78 aa, synthetic construct
  REGION 1..78: note = Synthetic peptide
GSNDEELKKILETLDRILKKLEKILTRLIEVLKKSEDPNLDDKDYTELVKQFIELIKKFEEVIKEYEEVVRQLIRLFS

SEQ ID NO: 37 - protein, 81 aa, synthetic construct
  REGION 1..81: note = Synthetic peptide
SKEEIDKIVKKHKKKIEEHKKKVDEHKKLVEEHDKRVSQDKDDKVKKLSEELKKISKRLEEVSKRLEEVSKKLLKVISDKR

SEQ ID NO: 38 - protein, 78 aa, synthetic construct
  REGION 1..78: note = Synthetic peptide
GSNDEELKKILETLDRILKKLDKILTRLDEVLKKSEDPNLDDKDYTELVKQYIELVKKYEEVVKEYEEVVRQLIRLFS

SEQ ID NO: 39 - protein, 69 aa, synthetic construct
  REGION 1..69: note = Synthetic peptide
DHSRKLKEILDRLRKHVKRLKEHLDELRDLVRQVPEDKLLEHVVKLSDKILQISERAVREFTKSVDKDS

SEQ ID NO: 40 - protein, 76 aa, synthetic construct
  REGION 1..76: note = Synthetic peptide
GSDKKDELERILDEIRRLIERLDEILSRLNKLLELLKHGVPNAKEVVKDYIRLLKEYLELVKEFLKLVKRHADLVS

SEQ ID NO: 41 - protein, 72 aa, synthetic construct
  REGION 1..72: note = Synthetic peptide
DSDEHLKKLKTFLENLRRHLDRLDKHIKQLRDILSENPEDERVKDVIDLSERSVRIVKTVIKIFEDSVRKKE

SEQ ID NO: 42 - protein, 75 aa, synthetic construct
  REGION 1..75: note = Synthetic peptide
GSDDKELDKLLDTLEKILQTATKIIDDANKLLEKLRRSERKDPKVVETYVELLKRHEKAVKELLEIAKTHAKKVE

SEQ ID NO: 43 - protein, 73 aa, synthetic construct
  REGION 1..73: note = Synthetic peptide
MDEEDHLKKLKTHLEKLERHLKLLEDHAKKLEDILKERPEDSAVKESIDELRRSIELVRESIEIFRQSVEEEE

SEQ ID NO: 44 - protein, 74 aa, synthetic construct
  REGION 1..74: note = Synthetic peptide
GDVKELTKILDTLTKILETATKVIKDATKLLEEHRKSDKPDPRLIETHKKLVEEHETLVRQHKELAEEHLKRTR

SEQ ID NO: 45 - protein, 72 aa, synthetic construct
  REGION 1..72: note = Synthetic peptide
DSDEHLKKLKTFLENLRRHLDRLDKLLKELRDILSENPEDERVKDVIDELERVIRIVKTVIKIFEDSVRKKE

SEQ ID NO: 46 - protein, 75 aa, synthetic construct
  REGION 1..75: note = Synthetic peptide
GSDDKELDKLLDTLEKILQTATKIIDDLNKVLEKLRRSERKDPKVIETVVELLKRHEKAVKELLEIAKTHAKKVE

SEQ ID NO: 47 - protein, 72 aa, synthetic construct
  REGION 1..72: note = Synthetic peptide
DSDEHLKKLKTFLENLRRLEDLLDKHIKQLRDILSENPEDERVKDVIDLSERVVRTVKTVIKIFEDSVRKKE

SEQ ID NO: 48 - protein, 75 aa, synthetic construct
  REGION 1..75: note = Synthetic peptide
GSDDKELDKLLDTLEKILQTATKVVDDANKLLEKLRRSERKDPKVVETYVELLKRLEKLIKELLEIAKTHAKKVE

SEQ ID NO: 49 - protein, 37 aa, synthetic construct
  REGION 1..37: note = Synthetic peptide
DSDEHLKKLKTFLENLRRHLDRLDKHIKQLRDILSEN

SEQ ID NO: 50 - protein, 114 aa, synthetic construct
  REGION 1..114: note = Synthetic peptide
EDERVKDVIDLSERSVRIVKTVIKIFEDSVRKLEKTKPDSKTAKELDKLLDTLEKILQTATKIIDDANKLLEKLRRSERKDPKVVETYVELLKRHEKAVKELLEIAKTHAKKVE

SEQ ID NO: 51 - protein, 114 aa, synthetic construct
  REGION 1..114: note = Synthetic peptide
DSDEHLYKLKTFLENLRRHLDRLDKHIKQLRDILSENPEDERVKDAIDLSERSVRIVKTVIKIFEDSVRKKEKRPIDKRDDKELDKLLDTLEKILQTATKIIDDANKLLEYLRR

SEQ ID NO: 52 - protein, 35 aa, synthetic construct
  REGION 1..35: note = Synthetic peptide
GDPKVVETYVELLKRHEKAVKELLEIAKTHAKKVE

SEQ ID NO: 53 - protein, 72 aa, synthetic construct
  REGION 1..72: note = Synthetic peptide
DSDEHLDRLDKHLKKLKTFLENLRRHIKQLRDILSENPEDERVKDVIDLSKTVIKIFEDSVRKKERSVRIVE

SEQ ID NO: 54 - protein, 75 aa, synthetic construct
  REGION 1..75: note = Synthetic peptide
GSDDKEATKIIDDLDKLLDTLEKILQTANKLLEKLRRSERKDPKVVETYVKAVKELLEIAKTHAELLKRHEKKVE

SEQ ID NO: 55 - protein, 72 aa, synthetic construct
  REGION 1..72: note = Synthetic peptide
DSDEHIKQLRDHLDRLDKHLKKLKTFLENLRRILSENPEDERVKTVIKIFEDSVRKKERSVRIVKDVIDLSE

SEQ ID NO: 56 - protein, 75 aa, synthetic construct
  REGION 1..75: note = Synthetic peptide
GSDDKEANKLLEKATKIIDDLDKLLDTLEKILQTLRRSERKDPKAVKELLEIAKTHAELLKRHEKVVETYVKKVE

SEQ ID NO: 57 - protein, 104 aa, synthetic construct
  REGION 1..104: note = Synthetic peptide
DHSRKLEEILDRLRKHVKRLLEHLRELLSLVKENPEDKDLVEVLELSLAILRRSLEAVEAFLKSVTKKDPDDEDLRRKADEIRKEVEEIKKSLAEVEKEIYKLK

SEQ ID NO: 58 - protein, 116 aa, synthetic construct
  REGION 1..116: note = Synthetic peptide
GSSADDVLEDILKIIRELIEILDQILSLLNQLLKLLRHGVPNAKKVVEKYKEILELYLQLVSLFLKIVKTHADAVSGKIDKKAEEEIKKEEEKIKEKLRQAKDILKKLQEEIDKTR

SEQ ID NO: 59 - protein, 107 aa, synthetic construct
  REGION 1..107: note = Synthetic peptide
DRDAHLYKLLTFLEQLVRHLDRLVKHITQLRDIVKKDPEDERAVDVIRQSVRSLEIVITVLKIFVDSVSDAARSKEAEKIVRKIRKEIDEIRQKLREIDKEVKKTTS

SEQ ID NO: 60 - protein, 114 aa, synthetic construct
  REGION 1..114: note = Synthetic peptide
GSNDKVLDKILDILDRILRLATRVIDLANKLLQVKKKSTHKDPRIVETYKELLKIHETAVRLLLELADLHRRLKSKDEEANKRVETELDRIRKKVKDIEDKVRKLEDKVRKTAS

SEQ ID NO: 61 - protein, 68 aa, synthetic construct
  REGION 1..68: note = Synthetic peptide
NDLSKEVLKKLEKSVEELLRRVQKSVKEAQKRGLLSDELVDRHLKILNQLVKRHLELLQEVIKRSDKK

SEQ ID NO: 62 - protein, 78 aa, synthetic construct
  REGION 1..78: note = Synthetic peptide
GSDEAVKRVVEKSLKILDEVIKKSLDILRELIELQIRHAKDDESVIRASKSALKDAIEALKKSLDEIKKALKRSADEG

SEQ ID NO: 63 - protein, 76 aa, synthetic construct
  REGION 1..76: note = Synthetic peptide
SSEEVVKVHEKVVKLHKEILELLKKIIKIHETAARDPDDKDSIKKLSDEIKKIVKRIEDISDQAKRESSDAQRKQS

SEQ ID NO: 64 - protein, 74 aa, synthetic construct
  REGION 1..74: note = Synthetic peptide
DKEEESKELLKKLKEILKRSEELLEESKELLKLAKNGEIDESELADADRKLNKKHEKLVQDIQDLLREHERQDR

SEQ ID NO: 65 - protein, 73 aa, synthetic construct
  REGION 1..73: note = Synthetic peptide
DEKKKIDKIVKETEDLLQKSEKLLQQSKEAVKRIRSQVKENEIVDRLLRISEELLKISRRLVEISRRIASTLS

SEQ ID NO: 66 - protein, 78 aa, synthetic construct
  REGION 1..78: note = Synthetic peptide
GSSKEEVIRLLKENVRLIKENLELLTRNLKLITDLVRGSNGSEEKIKTLKELLKEYRELLKRYRKLVEDYKRLVDKHD

SEQ ID NO: 67 - protein, 81 aa, synthetic construct
  REGION 1..81: note = Synthetic peptide
EIQELIKSSRRIIEESKELIKESEEVLRRIKEILDRIRNGVDNQEDLLREILKLLTKNLKIIQRNLKLLQDNAEILKRLVS

SEQ ID NO: 68 - protein, 75 aa, synthetic construct
  REGION 1..75: note = Synthetic peptide
GSYIEDVIKKILDVSRELIKLSRTIIKISEEINKQLQQGRDTKDLVKKYDEIIKKYTRIVQHYTELIKELQKLLS

SEQ ID NO: 69 - protein, 75 aa, synthetic construct
  REGION 1..75: note = Synthetic peptide
SPTEEAIQLSQRVIELSKRVIELSKEILKLLKRVLDLLPDLDKNEEKRLDDYDKELKEYDKELKKYEKRLKDLAS

SEQ ID NO: 70 - protein, 75 aa, synthetic construct
  REGION 1..75: note = Synthetic peptide
GSEEEEILKIQKELLRIQSEILDKQKKILDTLRSNGAVTEEVRSILEKVERLSEEAKELSKEAKELTKEVSKLIS

SEQ ID NO: 71 - protein, 77 aa, synthetic construct
  REGION 1..77: note = Synthetic peptide
SPLKELNNQLLRLLRELVKVSKKIVDLSKTIIEVLKHTDLDPRLLDSLEKSQQELDKSQKELDKVVKELTKVNKKLQ

SEQ ID NO: 72 - protein, 78 aa, synthetic construct
  REGION 1..78: note = Synthetic peptide
GSPLEDLVRKYDELVKTYEKLVEEFKKAVDKYDKAVKKAPVSKEATDSLDLIRKVLELLDRNLKLIKENAKLIKELLK

SEQ ID NO: 73 - protein, 69 aa, synthetic construct
  REGION 1..69: note = Synthetic peptide
SPTRENEKVIKENEKVISDNERVLEEVVKVVETATDRKEIQDAVDEVRKSVDKLRDSVRKLEESVRTLD

SEQ ID NO: 74 - protein, 72 aa, synthetic construct
  REGION 1..72: note = Synthetic peptide
GSPIKDISKRLLEISKRLVEISDRIVELLQRIADSKDPNKDLQKEVKDVLEEYKRLVREYREVVKEYEKVVS

SEQ ID NO: 75 - protein, 75 aa, synthetic construct
  REGION 1..75: note = Synthetic peptide
DEDEHVKQLIKNADLLRKHAELLKELVKLFQEIASQIPDDRVAKKVTDVVDRIDKILKQTEKLVRRTKQILDYSR

SEQ ID NO: 76 - protein, 71 aa, synthetic construct
  REGION 1..71: note = Synthetic peptide
GSNLEELVKLLKEVLEMHERLLRIHEDLVEAHKSNASDKESERKLKKSDKDIKESLKKIKSIIDQVRYIQS

SEQ ID NO: 77 - protein, 70 aa, synthetic construct
  REGION 1..70: note = Synthetic peptide
PVEDIIEESLRLLEESLKLLNRILKLLEDSLRKLPRSEEWRQRLDEFRKKLEDWKEELERWIEDVRYKKT

SEQ ID NO: 78 - protein, 78 aa, synthetic construct
  REGION 1..78: note = Synthetic peptide
GSDEDYESREIIDEIRKLLDRSKKIVHRSQRLVERVKSTPLSEDQEDLIRRHEETINRHRELVKELEKVLEDHERHIR

SEQ ID NO: 79 - protein, 78 aa, synthetic construct
  REGION 1..78: note = Synthetic peptide
PEEDSRRVLERFVRVSREVLKVLEEFLRVSEELLREADRDRDRRLEEYERQVDELREEIRRYKEEVDKFDKEVKYYKK

SEQ ID NO: 80 - protein, 79 aa, synthetic construct
  REGION 1..79: note = Synthetic peptide
GSPEKDENRKLLDKVRKLVEKSRRLVEELRKLVDQSTKNGLIDEKALRKQQEVLRKVEEVLEKQERVLRELEEISYRVI

SEQ ID NO: 81 - protein, 39 aa, synthetic construct
  REGION 1..39: note = Synthetic peptide
GSPERDENRKLLDKVRKLVEKSRRLVEELRKLVDQSTKN

SEQ ID NO: 82 - protein, 124 aa, synthetic construct
  REGION 1..124: note = Synthetic peptide
GSDEKALRKQQEVLRKVEEVLEKQERVLRELEEISYRVITRGEDHKAEEDSRRVLERFVRVSREVLKVLEEFLRVSEELLREADRDRDRRLEEYERQVDELREEIRRYKEEVDKFDKEVKYYKK

SEQ ID NO: 83 - protein, 39 aa, synthetic construct
  REGION 1..39: note = Synthetic peptide
GSDRRLEEYERQVDELREEIRRYKEEVDKFDKEVKYYKK

SEQ ID NO: 84 - protein, 125 aa, synthetic construct
  REGION 1..125: note = Synthetic peptide
GSPERDENRKLLDKVRKLVEKSRRLVEELRKLVDQSTKNGLIDEKALRKQQEVLRKVEEVLEKQERVLRELEEISYRVITRGEDHKAEEDSRRVLERFVRVSREVLKVLEEFLRVSEELLREADR

SEQ ID NO: 85 - protein, 75 aa, synthetic construct
  REGION 1..75: note = Synthetic peptide
DLSEESKKFVEKVKKLEKESRELEKQVKKIEEDSRSVENDVQKEFLELLKRLLDIQKKVVEVLREVVKVQQYVDS

SEQ ID NO: 86 - protein, 80 aa, synthetic construct
  REGION 1..80: note = Synthetic peptide
GSDSEYESRQVLRELDTVLKDSHTVLEALRQVIRDSQDVVSKSDEESRRVIDDLEKVIQDSKKVLDDIKRLIDKSKSIKS

SEQ ID NO: 87 - protein, 75 aa, synthetic construct
  REGION 1..75: note = Synthetic peptide
NEDELLKLLTENLKLLDENLKLLRENLSLLRQANNITDKNRIREIVKQSKEIVKQSREILKQSKEIVERIKYIVS

SEQ ID NO: 88 - protein, 78 aa, synthetic construct
  REGION 1..78: note = Synthetic peptide
GSSLYELTQRYEKLVQQYEELVKDYRRLVKKLEKLKRDNKPDKRLLKEIVDVIKKSVEIIDRSLKLLEESIKILEETD

SEQ ID NO: 89 - protein, 73 aa, synthetic construct
  REGION 1..73: note = Synthetic peptide
SQERSLEILKRILDVLKESLEILKESLSILRQLASRIKNPNRKIEEILKESDKIIKESDKVLKEIEEVIRYSS

SEQ ID NO: 90 - protein, 78 aa, synthetic construct
  REGION 1..78: note = Synthetic peptide
GSDIEYESKEILELIKELLKLSRELLKESRRALELVRKSRDDSIVEEVIQVHKKVLDIHKEVLKIVRKVVEVHRRVKS

SEQ ID NO: 91 - protein, 76 aa, synthetic construct
  REGION 1..76: note = Synthetic peptide
SKKDESTKLERLAEKIDEITKRIEELVKDVKRKSSEGVDKDQQQKIDEVFQKLLDLQREILEILDRILKVQQYILD

SEQ ID NO: 92 - protein, 78 aa, synthetic construct
  REGION 1..78: note = Synthetic peptide
GSDLEYLNRRLLQLIKTLIDLNRHLLKLIDKLKKLNSREGDEEKIKEESKQIQEQFKEIVERSKEIIKQIKEIIKRSQ

SEQ ID NO: 93 - protein, 79 aa, synthetic construct
  REGION 1..79: note = Synthetic peptide
DFERSSRRLEKVVEDLRRSSDRLREVIDELRKSADEKDEDEDLRRARKEHRDLIEELKRALEKQEEIIKHLQELVYRQL

SEQ ID NO: 94 - protein, 78 aa, synthetic construct
  REGION 1..78: note = Synthetic peptide
GSEESEEVRKVVERIKKISRELEEVVKELDRVSKEFDRHGETDEIVREHERIVEKLEEIVKKHTKIVEELAEIVYKQQ

SEQ ID NO: 95 - protein, 71 aa, synthetic construct
  REGION 1..71: note = Synthetic peptide
SDDDSVRVLDEIVKILDESVKLLKESLKLLDDFLRTKPDDHLKEVVKESKKVVEQSKKVLDRIKKIIYESK

SEQ ID NO: 96 - protein, 72 aa, synthetic construct
  REGION 1..72: note = Synthetic peptide
GSDLLYLSKELLKLVRELLKLSRELVELSRRLVNSTHKSPELVKKYDKLVKKYQDLLKKLADVADEYLRQRS

SEQ ID NO: 97 - protein, 76 aa, synthetic construct
  REGION 1..76: note = Synthetic peptide
DEKDYHRRLIEHLEDLVRRHEELIKRQKKVVEELERRGLDERLRRVVDRFRRSSERWEEVIERFRQVVDKLRKSVE

SEQ ID NO: 98 - protein, 78 aa, synthetic construct
  REGION 1..78: note = Synthetic peptide
GSDAYDLDRIVKEHRRLVEEQRELVEELEKLVRRQEDHRVDKKESHEILERLERIIRRSTRILTELEKLTDEFERRTR

SEQ ID NO: 99 - protein, 77 aa, synthetic construct
  REGION 1..77: note = Synthetic peptide
DERYRAREHIRRVEEHTKRLRHILKRLREHEEKLRRELKPGDEITESVDRFKKIVDQFEESIKKFETVSEELRKSDS

SEQ ID NO: 100 - protein, 71 aa, synthetic construct
  REGION 1..71: note = Synthetic peptide
GSDRQRILDRLDKILEKLDDILKKLKDILETLSKDDVSDRRHKDLVEKFRELVDTHHKLVERYRELVYQNR

SEQ ID NO: 101 - protein, 38 aa, synthetic construct
  REGION 1..38: note = Synthetic peptide
GSDEITESVDRFKKIVDQFEESIKKFETVSEELRKSIS

SEQ ID NO: 102 - protein, 107 aa, synthetic construct
  REGION 1..107: note = Synthetic peptide
GSDPQRAADRLDKILEKLDDILKKLKDILETLSKDDVKDRRAKDLVEKFRELVDTHHKLVERYRELVYTATAGSDLARELIRRVEEHTKRLRHILKRLREHEEKLRR

SEQ ID NO: 103 - protein, 73 aa, synthetic construct
  REGION 1..73: note = Synthetic peptide
NADDQLATSIKKLEDSIDQLIKIVRKFEESVKKLQKHGVDQHHVEILRKIVEIFRQHIEKLKKHLEKLRYTSS

SEQ ID NO: 104 - protein, 70 aa, synthetic construct
  REGION 1..70: note = Synthetic peptide
GSDKEYLVTEHEKLVREHEKIVSEIEKLVKKHEAGVDESELEEILKKVEKLLRKLDEILEQLTQLLRKTE

SEQ ID NO: 105 - protein, 36 aa, synthetic construct
  REGION 1..36: note = Synthetic peptide
GSDQHVVEILRKIVEIFRQHIEKLKKHLEKLRYTSS

SEQ ID NO: 106 - protein, 114 aa, synthetic construct
  REGION 1..114: note = Synthetic peptide
GSDAEYLVTEHEKLVREHEKIVSEIEKLVKKHEKGVDESELEEILKKVEKLLRKLDEILEQLTQLLRKAEKHIDKHSKAADQLATSIKKLEDSIDQLIKIVRKFEESVKKLQKH

SEQ ID NO: 107 - protein, 74 aa, synthetic construct
  REGION 1..74: note = Synthetic peptide
DEDDDIRRVLDESRRVLEHSRRVLKRSEEVLEKASRKKEKDTEEIEKHLKRLREHAKKLEKHRRELDDFLYKEI

SEQ ID NO: 108 - protein, 74 aa, synthetic construct
  REGION 1..74: note = Synthetic peptide
GSRDKYLLERLNDILKKLDEIVDKLSDILKRLKDVRHDDRLQELVERYKEIVKEYKRIVEEYEKLVREFEEQQR

SEQ ID NO: 109 - protein, 73 aa, synthetic construct
  REGION 1..73: note = Synthetic peptide
DRDYEDKEFKKIIKELEDVQEELKKLQEKIKRFSSELEEPNELLKEQLKVNEEQLEVNKKILKILRDQLKQNE

SEQ ID NO: 110 - protein, 76 aa, synthetic construct
  REGION 1..76: note = Synthetic peptide
GSDAEYKVRESVKRSKESVKHSEDVVDKLNKSVKLSESGHSDAEKASRELVKLVREVVELSREVIKLSEKVLRVIS

SEQ ID NO: 111 - protein, 79 aa, synthetic construct
  REGION 1..79: note = Synthetic peptide
DLQYKQEKLIRHFDRVVREWDKLVRKFSKVLEKQKHESKDKELEEASRRVDELIKRLREQLKRSKEILRRLKELSRKSS

SEQ ID NO: 112 - protein, 79 aa, synthetic construct
  REGION 1..79: note = Synthetic peptide
GSDWEELLRRLEKVLQEYEEIVKELIDLIERLIKVSEDKSKDASEYKKLVTELEKLISKLEEISKKLEELVKEYEYKTE

SEQ ID NO: 113 - protein, 76 aa, synthetic construct
  REGION 1..76: note = Synthetic peptide
DAKDELEKSLQEIEESLKELKKLLEELDKSLRELTSQGRNKKLEEHIKKVQKFIELVKKYIKAVQDYLKEVRYDNS

SEQ ID NO: 114 - protein, 75 aa, synthetic construct
  REGION 1..75: note = Synthetic peptide
GSDKERAARATEEMVKLTKKLLKAVEDLVRDVRRLLKEGLISEKHARIAETILEVFKKHAKIIKKHVDIVKYDES

SEQ ID NO: 115 - protein, 78 aa, synthetic construct
  REGION 1..78: note = Synthetic peptide
GSPLKERLLEIQRDLDRVLEEVVERLLRIQERLDSVVERKPPDVHEEYKYIVDEIREIVERVVREYEEIVKRIDEEVR

SEQ ID NO: 116 - protein, 83 aa, synthetic construct
  REGION 1..83: note = Synthetic peptide
GSEEDERIRYDLDRIRKDVRRKLEEIRQRVRELEKKLRDAGHRRDEKELLRELIETSKDILRLVEELLKKIIDKSEDLLRKTE

SEQ ID NO: 117 - protein, 73 aa, synthetic construct
  REGION 1..73: note = Synthetic peptide
GSDEEDYINENVEKDVRDIEDDVRRINERIRELLEKIRTEEVLQRVLEEHHELVERVLRKLVEILRKHEEENR

SEQ ID NO: 118 - protein, 77 aa, synthetic construct
  REGION 1..77: note = Synthetic peptide
GSDEEEYYKEKLHKLLREIEELLKHYRELVRRLEELVKRGELDKDTAAHILERLSELLERIIRRVAHTLRRLSEERR

SEQ ID NO: 119 - protein, 84 aa, synthetic construct
  REGION 1..84: note = Synthetic peptide
GSDEDEISYDSKRRVEEIVRQAREKSEKSRKDIEDVAEVLRKGDVSEKEVVDELVKVLEEQVKVLREAVERLREVLKKQVDDVR

SEQ ID NO: 120 - protein, 82 aa, synthetic construct
  REGION 1..82: note = Synthetic peptide
GSDIVELVDHLLKRSLKLLEELAELVRRLLEKSTELLKRRTEEHKEEVVEESEYMVRELEERLRRVVDESEKLVRDADKHIR

SEQ ID NO: 121 - protein, 83 aa, synthetic construct
  REGION 1..83: note = Synthetic peptide
GSKEKDIVKTLVDLLRENLETLERLIEEVVRLLKENVDVRDEGRDDKDSERILRDIKRRIDEAAKESREIIERIEKEVEYRSR

SEQ ID NO: 122 - protein, 80 aa, synthetic construct
  REGION 1..80: note = Synthetic peptide
GSPEVDVLRRIVREILKASEELLRLLRKLIDEALKLSERKRDSQEYREVVDRVKKELERLLDEYRKLVEELKEKLRYDTR

SEQ ID NO: 123 - protein, 84 aa, synthetic construct
  REGION 1..84: note = Synthetic peptide
GSDKRYESEKLKRRLDEAVEKVREVVERVERESDRVLEEVRRRRESKEVVDKVIEDNDKALEDVLRVVDEVAKVVRDVVRENTR

SEQ ID NO: 124 - protein, 77 aa, synthetic construct
  REGION 1..77: note = Synthetic peptide
GSPREYHSKDILRKVDEILERIRRHADRVKKKSERLKRENVDVNEHSKDVKRVIRELLELVKELLRLAKKHSDDQQE

SEQ ID NO: 125 - protein, 79 aa, synthetic construct
  REGION 1..79: note = Synthetic peptide
GSDEDEILYHSERLLQKLKKELDDLKEKSRELLEELKKEDPDDRLIERIIRLHDEVLKDLDEVLKNILEVHREVLERLR

SEQ ID NO: 126 - protein, 75 aa, synthetic construct
  REGION 1..75: note = Synthetic peptide
DKLDRLLKIHEEALRRAEELIKRLLDIHRRALDLARRGELDDYLLKESERELREIIRRAREELKESRDRLEEISR

SEQ ID NO: 127 - protein, 80 aa, synthetic construct
  REGION 1..80: note = Synthetic peptide
GSPKEELIRRVLEEVKRLNEKLLEIIRRAAELVKRANDELPETEKLREIDRELEKKLKEIEDELRRIDKELDDALYEIED

SEQ ID NO: 128 - protein, 80 aa, synthetic construct
  REGION 1..80: note = Synthetic peptide
GSPKLDKLRELLERNLEKLREILEEVLKILRTNLERVREDIRDEDVLQEYERLIRKAEEDLRRVLKEYDDLLKKLVYELR

SEQ ID NO: 129 - protein, 75 aa, synthetic construct
  REGION 1..75: note = Synthetic peptide
GSKEDESVKRAEEIVRTLLKLLEDSLREAERSLRDIKNGEDEHNLRRISEKLEELSKRITETIERLLRELQYTSR

SEQ ID NO: 130 - protein, 84 aa, synthetic construct
  REGION 1..84: note = Synthetic peptide
GSPNQELLDRVRKILEDLLRLNEELVRLNKELLKRALEMRRKNRDSEEVLERLAEEYRKRLEEYRRELEKLLEELEETIYRYKR

SEQ ID NO: 131 - protein, 84 aa, synthetic construct
  REGION 1..84: note = Synthetic peptide
GSDESEEAQHEVEKVLDDIRRLSEHLQKRLEEVLEEVYELRREGSDRTEVVELLKEVIREIVRVNREALERLLRVVEEAVKRNE

SEQ ID NO: 132 - protein, 83 aa, synthetic construct
  REGION 1..83: note = Synthetic peptide
GSDEEELVETVKRIQKEILDRLTELAKLLVEIQREIKKLKDEGEDDKELKRLSDELEEKVRQVVEEIKRLSDELEETVEYVSR

SEQ ID NO: 133 - protein, 80 aa, synthetic construct
  REGION 1..80: note = Synthetic peptide
GSDEEEEVVRRAEELVKEHEELIERVIRTHEELVYKLEDQGADKKLVDVLKRVVEESERVAREIVKVSRELIRLLEEASR

SEQ ID NO: 134 - protein, 80 aa, synthetic construct
  REGION 1..80: note = Synthetic peptide
GSSKEEILKELEDLQRRLIEELKKLQERVVELLEELIKRLRDRGRDDKHLKRLVKEVRRLSEEVLRSIKEVSDRVRYQLR

SEQ ID NO: 135 - protein, 81 aa, synthetic construct
  REGION 1..81: note = Synthetic peptide
GSDKEEESEYLLRDLVRLLEKVKEKIEEVNREVEKLLKKVKDGRLDRREVLREILRLNRELAEIIKEVVDRIRHVVERSER

SEQ ID NO: 136 - protein, 76 aa, synthetic construct
  REGION 1..76: note = Synthetic peptide
GSDLHEVVYETKELLKRIEEVVEELRKKSEDIIRKAERGEISEDELKRLQEEIAREAKKLLDEIKRVLERHLEQTL

SEQ ID NO: 137 - protein, 81 aa, synthetic construct
  REGION 1..81: note = Synthetic peptide
GSPVEEIIKEVVKRVIEVQEKVLRIISHAVKRVVEVQKKYDPGSEESNRVVEEVKKTIEDAIRESDEVVDEVVKRIQYTVR

SEQ ID NO: 138 - protein, 80 aa, synthetic construct
  REGION 1..80: note = Synthetic peptide
GSPEQEIADRILTEIRESQKELERLARKILKLLDESQEKAKRGRLSEEESDELLERIKKELDELLERSKELLKKIEYELR

SEQ ID NO: 139 - protein, 80 aa, synthetic construct
  REGION 1..80: note = Synthetic peptide
GSDEDKEANRVLDEVLKTVRDLLETANEVLKEVLYRLKRTDDQEKVVRTLTEVLKEHLKLVEEIVRILDKVLKEHLETEK

SEQ ID NO: 140 - protein, 76 aa, synthetic construct
  REGION 1..76: note = Synthetic peptide
GSPEDDVLRRLEEVSEKILRVAEDVARQLREVSEKITQGKVDRKEWEEDIKRLKRELEELLREWKEEIERLTYELR

SEQ ID NO: 141 - protein, 78 aa, synthetic construct
  REGION 1..78: note = Synthetic peptide
GSRREEVVKRIRELLKRNKELIDRIRELLEENEYLDKDARDKDVLRRSVELLEELVRILEESVELAKEIIKLLREVVE

SEQ ID NO: 142 - protein, 75 aa, synthetic construct
  REGION 1..75: note = Synthetic peptide
GSDEKEDNRRLQHKIERILEKNEDLQRKLEEILELLERGEADEEKIDRLRKAVEDYRRVVEEIKEDVKRHKYTVR

SEQ ID NO: 143 - protein, 77 aa, synthetic construct
  REGION 1..77: note = Synthetic peptide
GSDEKEEAKKASEESVRTVERILEELLKASEESVELLRRGEDAKDVVERSKEALKRVKELLDEVVKRSDEILKYIHN

SEQ ID NO: 144 - protein, 84 aa, synthetic construct
  REGION 1..84: note = Synthetic peptide
GSDEKKLINEVVETQKRLIKEAAKRLSEVVRHQTELIRELREKNVDDKDVEKLLKESLDLAEEIVRRIKELLDESKKLVEYVSN

SEQ ID NO: 145 - protein, 76 aa, synthetic construct
  REGION 1..76: note = Synthetic peptide
GSPDMDEVKRVLDELIEIQEEILREIKRVLEKLIKIQEDNGSEYESREVVREIVEIARKLVERSRRVVKKITETLQ

SEQ ID NO: 146 - protein, 80 aa, synthetic construct
  REGION 1..80: note = Synthetic peptide
GSDERYATREIVERIERIAREILKRTEEIVREVREVLSRDVDQEEVVRRLADLLRESVELVQHLVRRVEELLQESVERKK

SEQ ID NO: 147 - protein, 79 aa, synthetic construct
  REGION 1..79: note = Synthetic peptide
GSPEREALREVLEDLKRVTDRLRELVERVLEELKKVTDHVDSERILRESRRVLKELKDIIEEILRESEKVLEKLKYTED

SEQ ID NO: 148 - protein, 80 aa, synthetic construct
  REGION 1..80: note = Synthetic peptide
GSPAREILEEVVKKHLEVVEDAARILEEIIREHEKAVREDRDKKELEEISRDLLRKAREALKKVKDISDDLSREIEYVAS

SEQ ID NO: 149 - protein, 75 aa, synthetic construct
  REGION 1..75: note = Synthetic peptide
GSPVEEAIKKVIDDLRDVQRKIRELVEELIRLLEEVQRDNDKRESEYVVERVEEILRRITETSREVVRKAVEDLS

SEQ ID NO: 150 - protein, 81 aa, synthetic construct
  REGION 1..81: note = Synthetic peptide
GSDSDEKAEYLLKEMERVVRESDEVVKKILRDLEEVLERLRRGEISEDDVTEILKELAERHIRAIEELVRRLRELLERHKR

SEQ ID NO: 151 - protein, 79 aa, synthetic construct
  REGION 1..79: note = Synthetic peptide
GSPVEEVLKELSEVNERVRDIAREIIERLSEVNEEVKETDDEDELKKISKKVVDEVEDLLRKILEVSEEVVRRVEYHDR

SEQ ID NO: 152 - protein, 76 aa, synthetic construct
  REGION 1..76: note = Synthetic peptide
GSPKEDILREVLRRHKEIVREIVRLVREAVETHLELVKRNSDDRDAQDVIRKLEEDLERLVRHAQEVIEEIFYRLH

SEQ ID NO: 153 - protein, 75 aa, synthetic construct
  REGION 1..75: note = Synthetic peptide
GSPRSYLLKELADLSQHLVRLLERLVRESERVVEVLERGEVDEEELKRLEDLHRELEKAVREVRETHREIRERSR

SEQ ID NO: 154 - protein, 76 aa, synthetic construct
  REGION 1..76: note = Synthetic peptide
GSDREYIIKDILDSQEHLLRLIEELLETQKELLEILKRRPDSVERVRELVRRSKEIADEIRRQSDRNVRLLEEVSK

SEQ ID NO: 155 - protein, 75 aa, synthetic construct
  REGION 1..75: note = Synthetic peptide
GSDEKDEIRHVIESVERLIEDIKRLLKTLRELAHDDSDKKTVKEVLDRVKEMIERHRRELEEHRKELERAEYEVR

SEQ ID NO: 156 - protein, 76 aa, synthetic construct
  REGION 1..76: note = Synthetic peptide
GSESEDRIKELLKRHIELVERHEELLHEIKKLIDLEEKDDKDREEAVKRIDDAIKESEEMLEESKEILEEIEYLNR

SEQ ID NO: 157 - protein, 77 aa, synthetic construct
  REGION 1..77: note = Synthetic peptide
GSSLEDSVRLNDEVVKVVERVVRLNQEVVRLIKHATDVEDEETVKYVLERVREVLDESREVLKRVHELLEESERRLE

SEQ ID NO: 158 - protein, 82 aa, synthetic construct
  REGION 1..82: note = Synthetic peptide
GSHEKDIVYKVEDLVRKSDRIAERAREIVKRSRDIMREIRKDKDNKKLSDDLLKVTRDLQRVVDELEELSRELLRVAEESRK

SEQ ID NO: 159 - protein, 76 aa, synthetic construct
  REGION 1..76: note = Synthetic peptide
GSPELDEVKKLIDELKKSVERLEESIREVKESIKKLRKGDIDAEENIKLLKENIKIVRENIKIIKEIIDVVQYVLR

SEQ ID NO: 160 - protein, 82 aa, synthetic construct
  REGION 1..82: note = Synthetic peptide
GSDEEEIEELLRELEKLLKKSEEALEESKKLIDESEELLRRDRLDKEKHVRASEEHVKLSEEHLRISREIVKILEKAVYSTR

SEQ ID NO: 161 - protein, 79 aa, synthetic construct
  REGION 1..79: note = Synthetic peptide
GSDESDRIRKIVEESDEIVKESRKLAERARELIKESEDKRVSEERNERLLEELLRILDENAELLKRNLELLKEVLYRTR

SEQ ID NO: 162 - protein, 79 aa, synthetic construct
  REGION 1..79: note = Synthetic peptide
GSDEDDELERLLREYHRVLREYEKLLEELRRLYEEYKRGEVSEEESDRILREIKEILDKSERLWDLSEEVWRTLLYQAE

SEQ ID NO: 163 - protein, 78 aa, synthetic construct
  REGION 1..78: note = Synthetic peptide
GSDKKDASRRAIRVLHEFVRVSEEVLEVLRKSVESLKRLDVDEKIKRTHDRIEEELRRWKRELEELIERLREWEYHQD

SEQ ID NO: 164 - protein, 75 aa, synthetic construct
  REGION 1..75: note = Synthetic peptide
GSDDEEEDKRLLEEVKRSLDTDERILEKLRHSLERQLEDVDKDEDSRRVLRELDEITKRSREVVKRLRKLAYESK

SEQ ID NO: 165 - protein, 79 aa, synthetic construct
  REGION 1..79: note = Synthetic peptide
GSDKEYKLDRILRRLDELIKQLSRILEEIERLVDELEREPLDDKEVQDVIERIVELIDEHLELLKEYIKLLEEYIKTTK

SEQ ID NO: 166 - protein, 79 aa, synthetic construct
  REGION 1..79: note = Synthetic peptide
GSPSKEYQEKSAERQKELLHEYEKLVRHLRELVEKLQRRELDKEEVLRRLVEILERLKDLHKKIEDAHRKNEEAHKENK

SEQ ID NO: 167 - protein, 78 aa, synthetic construct
  REGION 1..78: note = Synthetic peptide
GSRDRKISEELIKALEDHIRMLEELIRAIEEHIKLAERGVDEKELRESLEELKKIVDELEKSLEELRKLAERYKYETR

SEQ ID NO: 168 - protein, 77 aa, synthetic construct
  REGION 1..77: note = Synthetic peptide
GSPKEESVEELKRVIDKHEEILRELKRVLEEHERVSHDEDENELRRSLERLKHILDRLHESLKELHELLKKNEYTER

SEQ ID NO: 169 - protein, 77 aa, synthetic construct
  REGION 1..77: note = Synthetic peptide
GSDHEYWVKIVERILRVMEKHAEIVKKHLEIVERVVREGPSEDLRRKLKESLREIEESLRELKELLDELDELSEKTR

SEQ ID NO: 170 - protein, 72 aa, synthetic construct
  REGION 1..72: note = Synthetic peptide
GSDEEYVTRSQRRLKRLLEEYIKVVEEHARLVERNERDDKELKRSIDELDKLTKELLELVKRYKELVDKTET

SEQ ID NO: 171 - protein, 76 aa, synthetic construct
  REGION 1..76: note = Synthetic peptide
GSDKEEIVKLQDEVIKTLERHLDILRKHIDLLEKLKDHLSEELKERVDRSIKKLEESIKRLERIIEELQELAEYSL

SEQ ID NO: 172 - protein, 75 aa, synthetic construct
  REGION 1..75: note = Synthetic peptide
GSREEELKESAEELERSVRELKKEADKYKEEVDRLHYRGKVDKDWVRVVEKLIKLVEEHLELIREHLELLKEERR

SEQ ID NO: 173 - protein, 75 aa, synthetic construct
  REGION 1..75: note = Synthetic peptide
GSDMEYELKKSAEELRKSLEELKRILDELHKSLRELRRHGDDEEYVQTVEELRKELEEHAKKLEEHLKELERVAT

SEQ ID NO: 174 - protein, 70 aa, synthetic construct
  REGION 1..70: note = Synthetic peptide
PEYELKKSVDDLKRDVDRLVEEVEEVFELSKERLREDRKHLELVEEMVRLIEKHLELIKEHLKLADDHVR

SEQ ID NO: 175 - protein, 78 aa, synthetic construct
  REGION 1..78: note = Synthetic peptide
GSREKDESKELNDEYKKLLEEYERLLRRSEELVKRAKGPRDEKELKRILEENEDILRRTKEILERTKEISEEQKYRRR

SEQ ID NO: 176 - protein, 78 aa, synthetic construct
  REGION 1..78: note = Synthetic peptide
GSDKDERQERLNEESDKSNEESERSNRESEELNRRARGPNDEKELQEILDRHLELLERNQRLLDENKEILRESQYLND

SEQ ID NO: 177 - protein, 76 aa, synthetic construct
  REGION 1..76: note = Synthetic peptide
GSENKYILKEILKLLRENLKLLHDILRLLDENLEELEKHGAKDLDDYRRKIEEIRKKVEDYREKIEEIEKKVERDR

SEQ ID NO: 178 - protein, 75 aa, synthetic construct
  REGION 1..75: note = Synthetic peptide
GSESEYTQEEILELLKESIKLLREILRLLEESEELWRRENTKSERSEEIKERAKEAIKRSEEILERVKRLSDHSR

SEQ ID NO: 179 - protein, 74 aa, synthetic construct
  REGION 1..74: note = Synthetic peptide
GSDEEEANYVSDKAVKIAEDVQELLKELLELSEVVRRGEVDEDEYDRVLRKLQEVMKEYEEVLKEYEEVSRKHE

SEQ ID NO: 180 - protein, 80 aa, synthetic construct
  REGION 1..80: note = Synthetic peptide
GSPEKYLIKTQEELLRRHAEILEDLIRKVERQVDLRRKVDERDEDLKRELERSLRELERLVRESSRLVEEIRELSKEIKR

SEQ ID NO: 181 - protein, 78 aa, synthetic construct
  REGION 1..78: note = Synthetic peptide
GSDEEYELERISRESKELLERYKRLLREYQELLKELRHVKDLDRAVKIIHELMRVSKELVEISHRLLELHERLVRRRK

SEQ ID NO: 182 - protein, 79 aa, synthetic construct
  REGION 1..79: note = Synthetic peptide
GSEKEYIEKLSRKIEEDIRRSEERAKDSERLVRRLEELAKRKRLDLDDVLRVAEENLEILEDNLRILEEILKEQDKSNR

SEQ ID NO: 183 - protein, 75 aa, synthetic construct
  REGION 1..75: note = Synthetic peptide
GSPHEEVVELHERVMEISERAVELIQRIIDIIRRIREDDKDIEKLVKTIRDLVREYEELHRELEEIDEEIYKKSE

SEQ ID NO: 184 - protein, 82 aa, synthetic construct
  REGION 1..82: note = Synthetic peptide
GSDHEDVVRLHEDLVRKQEDARRVLEEIVRLAEEIVEVIKKDEKDKDRVTRLVEEIEKLVEEYKKKVDEMRKISDEIKYRSR

SEQ ID NO: 185 - protein, 79 aa, synthetic construct
  REGION 1..79: note = Synthetic peptide
GSRAREVVKRAKRIIEEWQKILEEWRRILEEWRRLLEDERVDDRDNERIIRENERVIRENEKIIRDVIRLLEELLYERR

SEQ ID NO: 186 - protein, 79 aa, synthetic construct
  REGION 1..79: note = Synthetic peptide
GSREDEELEEEIDRIRQMVEEYEELVKEYEELTEKYKQGKVDKEESKKIIEKSERLLDLSQDAVRKVKEIIRRILYTNR

SEQ ID NO: 187 - protein, 77 aa, synthetic construct
  REGION 1..77: note = Synthetic peptide
GSPKEEIVKLHDESAELHRRSVEVADEILKMHERSKDVDDERESRELSKEIERLIREVEEVSKRIKRLSEEVEYLVR

SEQ ID NO: 188 - protein, 77 aa, synthetic construct
  REGION 1..77: note = Synthetic peptide
GSPLEEILKIQRRINKIQDDINKILHEILRMQEKLNRSSDKDEVEESLRRIRELIKRIKDLSKEIEDLSREVKYRTT

SEQ ID NO: 189 - protein, 81 aa, synthetic construct
  REGION 1..81: note = Synthetic peptide
GSPEDEHVYVVREIYEVLREHAEVLEENREVIERLLEAKKRGDKSEELVKELKKSIDKLKEISRKLEEIVKELEKVSEKLK

SEQ ID NO: 190 - protein, 79 aa, synthetic construct
  REGION 1..79: note = Synthetic peptide
GSDEDETSYRILELLREIVRASRELIRLSEELLEVARRDDKDETVLETLIREYKELLDRYRRLIEELTRLVEEYEERSR

SEQ ID NO: 191 - protein, 74 aa, synthetic construct
  REGION 1..74: note = Synthetic peptide
GSTQEEINRIQHEVLRIQEEIDEILRDIVEKLKAISRGELDHEVVKDVEDKVREALEKSEELLDKSRKVEYKSE

SEQ ID NO: 192 - protein, 82 aa, synthetic construct
  REGION 1..82: note = Synthetic peptide
GSDEEELNRELLEKSKRLVDINRDIIRTAQELIEMLKDSKDGRVDEDTKRELRDKLRKLEEKLERVREELRKYEELLRYVQR

SEQ ID NO: 193 - protein, 77 aa, synthetic construct
  REGION 1..77: note = Synthetic peptide
GSDEKDRVYEILKEVQRLVKEYRDISKEIEDLVKHYEHITDDEAQEVSKELIDKSLRASEIVRELIRLIKELLDELE

SEQ ID NO: 194 - protein, 79 aa, synthetic construct
  REGION 1..79: note = Synthetic peptide
GSDEEDVLYHLRELLEELKRVSDDYERLVREIKETSERKDRDTKENKDMLDELVKAHREQEKLLERLVRLLEELFERKR

SEQ ID NO: 195 - protein, 54 aa, synthetic construct
  REGION 1..54: note = Synthetic peptide
PREQAIRISEEIIRISKKIIEILERTRSSTAREAMKWAKDSIRLAEESKYLLDK

SEQ ID NO: 196 - protein, 54 aa, synthetic construct
  REGION 1..54: note = Synthetic peptide
IEDDVKKIQDSTKKAQKETIEALERSTSSTARKQMEEQKEQIRLQKEAMYLLKK

SEQ ID NO: 197 - protein, 76 aa, synthetic construct
  REGION 1..76: note = Synthetic peptide
SREEIAKLQEEVIKLQRRVIELQKEVIELQRRAKELTSSYTKEILEIQRRIEEIQREIEEIQKRIEEIQEEIQRRT

SEQ ID NO: 198 - protein, 76 aa, synthetic construct
  REGION 1..76: note = Synthetic peptide
SDEEIKRLSEEVIQLSRRVIKMSREAIKLSREVQKLTPSYQKRIKEIADRSIELARESIEIAKRSEKIAEESQRRT

SEQ ID NO: 199 - protein, 56 aa, synthetic construct
  REGION 1..56: note = Synthetic peptide
PAKDEALKMANESLELAKKSARLIQESSSKEILERIEKIQRRIAELQDRIAYLIKK

SEQ ID NO: 200 - protein, 56 aa, synthetic construct
  REGION 1..56: note = Synthetic peptide
PAKDEALRMIDESRELIKKSNELIQRSSSKEILERILEIQRKIAELQKRIQYLLKS

SEQ ID NO: 201 - protein, 75 aa, synthetic construct
  REGION 1..75: note = Synthetic peptide
TDEARYRSERIVKEAKRLLDEARRRSEKIVREAKQRSNSEDAKRIMEENLRESEEAARRLREIIRRNLEESRETG

SEQ ID NO: 202 - protein, 75 aa, synthetic construct
  REGION 1..75: note = Synthetic peptide
TREALEYQRKMAEEIEDLLREALRRQEEMVREAKQRSLSEEFKRIMERILEEQERVMRLAKEALERILEEQKRTG

SEQ ID NO: 203 - protein, 76 aa, synthetic construct
  REGION 1..76: note = Synthetic peptide
SERTKREAKRSQEEILREAKEAMRRAKESQDHRQNRDGSNSEDLERLSQEQKRELEEVERRLKELAREQKYKLEDS

SEQ ID NO: 204 - protein, 76 aa, synthetic construct
  REGION 1..76: note = Synthetic peptide
SEDLKRILKEITERELKLMQDLMEILKKITEDENNLDSNNSEDLKRSIEKARRILDEALRKLEESARRAKYIQEDN

SEQ ID NO: 205 - protein, 76 aa, synthetic construct
  REGION 1..76: note = Synthetic peptide
TEDEIRESLKWLDEVLQELREIARESNEVLERNRQKSRSDKLREDIERYKKRMEEARKKLDDQLNKYKKRMDENRS

SEQ ID NO: 206 - protein, 76 aa, synthetic construct
  REGION 1..76: note = Synthetic peptide
TEEELKESKKFAEDLARSARRALKESKRVLEEISQASRSKKLEEIVRRYKEQVKRWQDEWDERAREYRKRMKENRS

SEQ ID NO: 207 - protein, 76 aa, synthetic construct
  REGION 1..76: note = Synthetic peptide
TKTEEIERLAREIKKLSEKVERLAQEIEELSRRVKEENSTDRELKEANREIERAIREIEKANKRMEEALRRMKYNG

SEQ ID NO: 208 - protein, 76 aa, synthetic construct
  REGION 1..76: note = Synthetic peptide
TKTEEHERLAREISKLADEHRKLAKIIEELARRIKEENLTDDELREAIRKIEDALRKNKEALKIMKEAAERNRYNT

SEQ ID NO: 209 - protein, 76 aa, synthetic construct
  REGION 1..76: note = Synthetic peptide
TKKEESRELARESEELARESEKLARKSLELARRAESSGSEEEKRRIIDENRKIIERNREIIERNKEIIEYNKELIS

SEQ ID NO: 210 - protein, 76 aa, synthetic construct
  REGION 1..76: note = Synthetic peptide
TKDEESLELNRESEELNRKSEELNRKSKELNDRAESSNSEEEEKEILREHKEILREHLEILRRHKEILRRHKYLTS

SEQ ID NO: 211 - protein, 75 aa, synthetic construct
  REGION 1..75: note = Synthetic peptide
TREELLRENIELAKEHIEIMREILELLQKMEELLERQSSEDILEELRKIIERIRELLDRSRKIHERSEEIAYKEE

SEQ ID NO: 212 - protein, 79 aa, synthetic construct
  REGION 1..79: note = Synthetic peptide
SEDIAREIKELLRRLKEIIERNQRIAKEHEYIARERKKLDPSNEKERKLLERSRRLQEESKRLLDEMAEIMRRIKKLLD

SEQ ID NO: 213 - protein, 75 aa, synthetic construct
  REGION 1..75: note = Synthetic peptide
DRQKLIEENIKLLDKHIKILEEILRLLKKDIDLLKKSSSEEVLEELKKIHRRIDKLLDESKKIHKRSSEIVKKRS

SEQ ID NO: 214 - protein, 75 aa, synthetic construct
  REGION 1..75: note = Synthetic peptide
DEQKLIETSQRLQEKSERLLEKFEQILREASDLYRKPDSEELLRRVEKLLRELEKLIRENQDLARKHEKILRDQS

SEQ ID NO: 215 - protein, 75 aa, synthetic construct
  REGION 1..75: note = Synthetic peptide
DRQELIRENIELLKKHIKIVKEIQKLIETFIELLKKSSSEEILRRLKKILKRIEKLYRESQEIHKRSEEIAKKRQ

SEQ ID NO: 216 - protein, 75 aa, synthetic construct
  REGION 1..75: note = Synthetic peptide
DEERLIDKSRELQKESEELLKELLKIFKRIEELLEKPDSEELIREIKKLLETLSEIHKRNEKLARTHEEILRQQS

SEQ ID NO: 217 - protein, 74 aa, synthetic construct
  REGION 1..74: note = Synthetic peptide
STRDVQREIAKAFKKMADVQKKLAEEIKRHVKNVEKKNKDNDEYRKIATELLKKATESQKKLKELLDRIRKSDS

SEQ ID NO: 218 - protein, 74 aa, synthetic construct
  REGION 1..74: note = Synthetic peptide
DKDDRSTSLLKRVEKLIDESDRIIDKFTTLIELSRNGKIDDDQYKKELKEILELLKKYDKHVKEVEELLKRLNS

SEQ ID NO: 219 - protein, 70 aa, synthetic construct
  REGION 1..70: note = Synthetic peptide
SKRKALEVSERVVRISEKVVRVLDESSDLLKKSYDDSDKFAELIDRHEEKIKKWKKLIKEWLEIIQRHKS

SEQ ID NO: 220 - protein, 68 aa, synthetic construct
  REGION 1..68: note = Synthetic peptide
SAEEFVKLSEEAVKRSKEILDIVRKQVKLVKAGVDKHEITDSLRKSEKLIEEHKELIKTHRDLLRREN

SEQ ID NO: 221 - protein, 74 aa, synthetic construct
  REGION 1..74: note = Synthetic peptide
SSTEILKRFKRALRESEKIVKHSRRVLKIIREVLKQKPTQAVHDLVRIIETQVKALEEQLKVLKRIVEALERQS

SEQ ID NO: 222 - protein, 73 aa, synthetic construct
  REGION 1..73: note = Synthetic peptide
DKQKEIKDILEKTRRIAEESRKIAEKFDEIIKRSTEGKIDESLTKELEELVKEVIKLSEDDARTSDDLVRKES

SEQ ID NO: 223 - protein, 74 aa, synthetic construct
  REGION 1..74: note = Synthetic peptide
DEDESIKLTRKSIEETRKSLKIIKEVVELIREVLKHIKDLDKEIFERIDKILDKYKKQVDTYDEILKEYEKKQR

SEQ ID NO: 224 - protein, 70 aa, synthetic construct
  REGION 1..70: note = Synthetic peptide
SELDEQKELIKKQEKLIEEQQRLLSKIRRMFKERVKDQELLREIQKVLKRSQEIVETSKKILDRSDKTTE

SEQ ID NO: 225 - protein, 76 aa, synthetic construct
  REGION 1..76: note = Synthetic peptide
DQKEINTRIVEKLERIFKKSKEIVRQSERVISTIEKKTEDERELDLLRRHVKIVREHLKLLEELLKIIKEVQKESE

SEQ ID NO: 226 - protein, 71 aa, synthetic construct
  REGION 1..71: note = Synthetic peptide
DTEELVKRLNELLKELSKLVKEFIKILETYRKDQTKDTSKISERVDRILKTYEDLLQKYKEILEKIEKQLS

SEQ ID NO: 227 - protein, 68 aa, synthetic construct
  REGION 1..68: note = Synthetic peptide
DYARLIDQAVEVTRKVVEVNVTVARVNDKFAKHLGDEELRRVSEHLKEVSKDLQEVAKKSKDAARQVK

SEQ ID NO: 228 - protein, 77 aa, synthetic construct
  REGION 1..77: note = Synthetic peptide
DVSKVAEEYLQISKTLVDISRTLLEISERLVRLVRTVADDRSEVKKAIEDSIEVLKTSEEVVRQIKRASDKLVKAIS

SEQ ID NO: 229 - protein, 76 aa, synthetic construct
  REGION 1..76: note = Synthetic peptide
DAKEIQRRVVEIQTEVVKLQKKAVDIIRKIIEAFNNSNIDQSLLEAAKEIVKEIDKLEKLTESLLEESKKLLKRSS

SEQ ID NO: 230 - protein, 76 aa, synthetic construct
  REGION 1..76: note = Synthetic peptide
SAEEVVKLAKIFLELLRESIKLLKRSVDLLRKSSDPSLDKSEAEKVSREIEKVSDTSLKLSKKALDVVKRALKVAS

SEQ ID NO: 231 - protein, 81 aa, synthetic construct
  REGION 1..81: note = Synthetic peptide
DEKDAARKARKVSEEAKEASKKIEKALEESKRILNTLKQKKDEQEVKVIKEHEDVLRQIEKIQKQVLEIQKEVAKLLESLD

SEQ ID NO: 232 - protein, 71 aa, synthetic construct
  REGION 1..71: note = Synthetic peptide
SADDVARASEKVLRVARESAKAADKSLEVFKEVVKRGDKEAFLQVVKINEEVVKINITVIRILIEVSKTAT

SEQ ID NO: 233 - protein, 112 aa, synthetic construct
  REGION 1..112: note = Synthetic peptide
DEYVKETLKQLREALASLREADKRITELVKEARKKPLSEAARKFAEAIVTHVKVVVEHVEVVLRHVEVLVEAKKNGVIDKSILDNALRIIENVIRLLSNVIRVVDEVLQDLD

SEQ ID NO: 234 - protein, 113 aa, synthetic construct
  REGION 1..113: note = Synthetic peptide
DASDVIRRIHELFEEVHRLIEAVHRAIEDVAKAAQKKGLDESAVEILAELSKELAKLSRRLAEISREIQKVVTDPDDKEAVERLKEIIKEIKKQLDELRDRLRKLQDLLYKLK

SEQ ID NO: 235 - protein, 77 aa, synthetic construct
  REGION 1..77: note = Synthetic peptide
SEDKAHHDIVRVLEELIKIHDELMKISEEILKATSDSTATDETKEELKRRSKEAQKKSDTLVKIVKELEKESRKAQS

SEQ ID NO: 236 - protein, 73 aa, synthetic construct
  REGION 1..73: note = Synthetic peptide
DDEEKYRQIIREAQEISKTAKRILRDAQEISKRIRHQGVDRSEHQRLVDLLRELIKEHHKLLRRQQEADTRND

SEQ ID NO: 237 - protein, 74 aa, synthetic construct
  REGION 1..74: note = Synthetic peptide
DRKDKARKASEKLEEVIQRWKTVADKWKKMVDLVSNGKLSQEEVARVTEELLKIQTELAKLLEEHAKVLQESAS

SEQ ID NO: 238 - protein, 77 aa, synthetic construct
  REGION 1..77: note = Synthetic peptide
SDEESIKTQSELIKTSEELLKDVKRIDEELQKLRDDPTLDESELKKRVKEWSDRVRKAKEISRKIQEIVKESKKRSS

SEQ ID NO: 239 - protein, 72 aa, synthetic construct
  REGION 1..72: note = Synthetic peptide
DKDEELRKVIEKYREMVKEYRKVIREYEEVIKSSKTIDKSSLISLSRKMVELSQRVIDVSDEVAKVLSRKQS

SEQ ID NO: 240 - protein, 69 aa, synthetic construct
  REGION 1..69: note = Synthetic peptide
TDEERLKKQTKELKEQTKQLEKQKDLLEKISNGEISKDEIQEIIKESKKIAKESQKALDSSRKALEEVS

SEQ ID NO: 241 - protein, 75 aa, synthetic construct
  REGION 1..75: note = Synthetic peptide
DEKEVSKEIIKVLKDIAKVQQKVIEVSQRLASVLRADDDNVVKRALEEYEKILEELRELNKEIEKLTDKYRKVTS

SEQ ID NO: 242 - protein, 77 aa, synthetic construct
  REGION 1..77: note = Synthetic peptide
DSDEQTKELEKLTELHKRHVEKLKKQTKESREVDSNKLWKSKDVKDKLSESEKELQKLSDQDKKAKDALESSRRKND

SEQ ID NO: 243 - protein, 76 aa, synthetic construct
  REGION 1..76: note = Synthetic peptide
DAEEQLKLLTKLLRHQQRLLQLIKESLKLIEKIDQSSQENQDEIRKWREVTKKLRELIKTSEKLVRELEKSYKKSS

SEQ ID NO: 244 - protein, 70 aa, synthetic construct
  REGION 1..70: note = Synthetic peptide
SLRDVVRRYQELVRRYDELIKTLTEILKKYQKKGAEDKDASTELVKAVRTSLKLSKELLKLNSELLKEDS

SEQ ID NO: 245 - protein, 69 aa, synthetic construct
  REGION 1..69: note = Synthetic peptide
SKEELKRKLDELKKRSDTLKELSKKLKEISERNPDDKSVHRTIIRIHREFVKNHKEIVRVIEEIVSDKS

SEQ ID NO: 246 - protein, 71 aa, synthetic construct
  REGION 1..71: note = Synthetic peptide
SKQDEHDRLLKIHDKLVKQHDELLKLLTKLSRAGDSVTKKKLEEILRKLQEVSKQLEESLKDADKVSKDIN

SEQ ID NO: 247 - protein, 69 aa, synthetic construct
  REGION 1..69: note = Synthetic peptide
TVQSLLEQHVKIVKRSIEILERHTQILQDIARSQGVSKELEDVERQVKEYRKEVKKLEEDLRQLSRNSK

SEQ ID NO: 248 - protein, 76 aa, synthetic construct
  REGION 1..76: note = Synthetic peptide
SDSDRIEKLIRESTELLKEQQKLAKRSRELAETVESLPLTEEYLKQQREHQKKIEKLLKDSEKHLEELKRLVKSEK

SEQ ID NO: 249 - protein, 70 aa, synthetic construct
  REGION 1..70: note = Synthetic peptide
DSEKRIEDILRTDLELAKRDAELVKEHIKLVKRIDLSEELKKQVEDVEKESKKLEDSSEKLVQKVRKRSS

SEQ ID NO: 250 - protein, 80 aa, synthetic construct
  REGION 1..80: note = Synthetic peptide
DEEERAKDLRKYLEEQTQYYRTVTEHLRNLEKVVEELERRGKPSSELQQILERSQRIYKETTEIYDTSKKLIEELDKHHR

SEQ ID NO: 251 - protein, 77 aa, synthetic construct
  REGION 1..77: note = Synthetic peptide
PLEDILKRHLDKVRELVRLSEEVNKLAKEVLDILKDKRVDEKELDKVLKELEKVVEEYERAVKESRDLLRELRETTR

SEQ ID NO: 252 - protein, 75 aa, synthetic construct
  REGION 1..75: note = Synthetic peptide
DKERLLEIHERIQKLLDRNLEIIERLLRLLREARDIKDDDKLDKVIKRLKELSEESKDILDKIKELLKESEKELT

SEQ ID NO: 253 - protein, 75 aa, synthetic construct
  REGION 1..75: note = Synthetic peptide
PEDEVIRVIEELLRIAAEVDEVHRRNVEVQEEASRVTDRERLERLNRESEELIKRSRELIEEQRKLIERLERLAT

SEQ ID NO: 254 - protein, 74 aa, synthetic construct
  REGION 1..74: note = Synthetic peptide
DLEELIKEYAEVVRRHHKAVRDLERLVRELANAKHASEEELKRIATEILRIVKELIRVQERLIKLSEDSNEESR

SEQ ID NO: 255 - protein, 75 aa, synthetic construct
  REGION 1..75: note = Synthetic peptide
PTDEVIEVLKELLRIHRENLRVNEEIVEVNERASRVTDREELERLLRRSNELIKRSRELNEESKKLIEKLERLAT

SEQ ID NO: 256 - protein, 74 aa, synthetic construct
  REGION 1..74: note = Synthetic peptide
DNEEIIKEARRVVEEYKKAVDRLEELVRRAENAKHASEKELKDIVREILRISKELNKVSERLIELWERSQERAR

SEQ ID NO: 257 - protein, 72 aa, synthetic construct
  REGION 1..72: note = Synthetic peptide
PKEDIDRVSRELVRVHKELLEVLRKSTEIVEAVARNEKDERTIEEVLEEQERAVRKLEEVSKKHKEAVKRLK

SEQ ID NO: 258 - protein, 77 aa, synthetic construct
  REGION 1..77: note = Synthetic peptide
ELERLSEEIQKLSDRLIELIRRHSKVLEEIVRLLKHKDNDEREVRRLLKLLRDLTRRYEEVLRKVEEIVKRQEDESR

SEQ ID NO: 259 - protein, 72 aa, synthetic construct
  REGION 1..72: note = Synthetic peptide
PEEDILRLLRKLVEVDKELLEVVRESTEVVRLVARNEKDVETVERVLRKQEEVVRKYERVSRELEEAVRRLK

SEQ ID NO: 260 - protein, 77 aa, synthetic construct
  REGION 1..77: note = Synthetic peptide
ELKDLVEEIVKLSKENLKLWEDHSRVLEEIVRLLKHKDNDEREVRRLLKLLEDLTRRAEETSRRIEEIVKEAEDRAR

SEQ ID NO: 261 - protein, 74 aa, synthetic construct
  REGION 1..74: note = Synthetic peptide
DEERELREVLRKHHRVVREWTKVVEELKRVVELLKRGETSEEDLLRVLKKLLEMDKRILEVNREVLRVLEKRLT

SEQ ID NO: 262 - protein, 70 aa, synthetic construct
  REGION 1..70: note = Synthetic peptide
SLEEIIEELVELVRRSVEIAKESDEVARRIVESEDKKKELIDTLRDLHREWQEVTKRAEELVREAEKEVR

SEQ ID NO: 263 - protein, 72 aa, synthetic construct
  REGION 1..72: note = Synthetic peptide
TAEELLEVHKKSDRVTKEHLRVSEEILKVVEVLTRGEVSSEVLKRVLRKLEELTDKLRRVTEEQRRVVEKLN

SEQ ID NO: 264 - protein, 76 aa, synthetic construct
  REGION 1..76: note = Synthetic peptide
DLEDLLRRLRRLVDEQRRLVEELERVSRRLEKAVRDNEDERELARLSREHSDIQDKHDKLAREILEVLKRLLERTE

SEQ ID NO: 265 - protein, 72 aa, synthetic construct
  REGION 1..72: note = Synthetic peptide
PEDDVVRIIKEDLESNREVLREQKEIHRILELVTRGEVSEEAIDRVLKRQEDLLKKQKESTDKARKVVEERR

SEQ ID NO: 266 - protein, 70 aa, synthetic construct
  REGION 1..70: note = Synthetic peptide
DEVRLITEWLKLSEESTRLLKELVELTRLLRNNVPNVEEILREHERISRELERLSRRLKDLADKLERTRR

SEQ ID NO: 267 - protein, 70 aa, synthetic construct
  REGION 1..70: note = Synthetic peptide
DEDEVVKVHEEHVKSHEEIHRSHEEVVRAAEEDKRDSRELRTLMEEHRKLLEENEKSIEEVKKIHERVKR

SEQ ID NO: 268 - protein, 80 aa, synthetic construct
  REGION 1..80: note = Synthetic peptide
KKEELIDISKEVLDLDDEINKISKEILELIKKLLRLKEEGREDKDKAREVKRRIRELHRRIQELNKRLRELHKRVQETKR

SEQ ID NO: 269 - protein, 77 aa, synthetic construct
  REGION 1..77: note = Synthetic peptide
PEEDIARRVEDLLRKSEELIKESEKILKESKRLLDRNDSDKRVLETNLRLIDKHTKLLERNLELLEELLKLAEDVAK

SEQ ID NO: 270 - protein, 74 aa, synthetic construct
  REGION 1..74: note = Synthetic peptide
RFKDLSREYIEVVKRLLELSREALEVLREIKDTDKTDKKRIKELIDRLRKLIEEYKRIIDRLRKLSKDLEEEHR

SEQ ID NO: 271 - protein, 73 aa, synthetic construct
  REGION 1..73: note = Synthetic peptide
DEEELVKILKELQRLSEESLEINKRLVEILRLLRRGEVPKEEVEKKLREIKKEQEKLDREHEKIKKRIEEITK

SEQ ID NO: 272 - protein, 74 aa, synthetic construct
  REGION 1..74: note = Synthetic peptide
SLKEKILEIIERNMKLVELSNRSVEIVARILKGEKDDEETLERLLREWDKITRDYEEIIKESRKLVKELEEEAK

SEQ ID NO: 273 - protein, 74 aa, synthetic construct
  REGION 1..74: note = Synthetic peptide
SKTEILRKALEIHKEQIDIVRKLIELSEEVLKLVEESKEKNLEKLKRIDEETDRLLERLDELHKRLTELAERLK

SEQ ID NO: 274 - protein, 80 aa, synthetic construct
  REGION 1..80: note = Synthetic peptide
SDDEARKQLEEMKRRLREVEKKSKRVEERVRELERLVRENREDEDRVLKTLEDLLRENEKLVRTIERHVREQRELSKEVK

SEQ ID NO: 275 - protein, 73 aa, synthetic construct
  REGION 1..73: note = Synthetic peptide
SEEELEKKADELRKLSEEWRKLQEEDKRLSEMVEKGELDLQEVDEHSLRVLERATEVHRTVDKVIEEILRTTN

SEQ ID NO: 276 - protein, 77 aa, synthetic construct
  REGION 1..77: note = Synthetic peptide
SEKERHRESQETQEEIRRTHEEIIRKLEEILRRAKAGELPEETLDRLRRIMERLKELSERLDDLVRKLRDDHRREQK

SEQ ID NO: 277 - protein, 76 aa, synthetic construct
  REGION 1..76: note = Synthetic peptide
SEKEILEELKRILKRVKDISDRLEELDKRTEEIARREPTKELVDELVKIHRDWLRLHEEILKLVDDALKKVEDATK

SEQ ID NO: 278 - protein, 73 aa, synthetic construct
  REGION 1..73: note = Synthetic peptide
DLRELLELQREASRLHRELVKLLTELVKKLELIAKGEDIREEDLKRIKERLEEIKKRSKRIKEESDEIDKKTK

SEQ ID NO: 279 - protein, 72 aa, synthetic construct
  REGION 1..72: note = Synthetic peptide
SERELQRELNKIVRRILEIHREVSELHQRAVKLIRENDNSEELEEISRRIEELSKELEKLVREHDEIVKTIE

SEQ ID NO: 280 - protein, 77 aa, synthetic construct
  REGION 1..77: note = Synthetic peptide
SEREKLDRNDEELKEINKRVEEIKERSDRITEAIEKNERSEEEIRRLSREQNEALQRLLELHKKLVKLHRELLEDTR

SEQ ID NO: 281 - protein, 76 aa, synthetic construct
  REGION 1..76: note = Synthetic peptide
DKEDVIRVHDEQHKLIEEQLELTRRIAELVREIAKNTASEEEIKEMLKEIKRLDDRSREIQDRLQKLLEEIRRKTK

SEQ ID NO: 282 - protein, 73 aa, synthetic construct
  REGION 1..73: note = Synthetic peptide
TEEEIVELNKDIQRKSKEHIDLQNELVKKIERAIRENNITEELLEELERLLRESEKIVEEIRRITDKIRKDAK

SEQ ID NO: 283 - protein, 72 aa, synthetic construct
  REGION 1..72: note = Synthetic peptide
SEKEILERLLRLSKEQNEISEEIHRLTERLVELKRRKDDDERLKRILDRQKRLVERAREISKEYEDLLRKLE

SEQ ID NO: 284 - protein, 69 aa, synthetic construct
  REGION 1..69: note = Synthetic peptide
SMEELLRKNARLSRKQLKIIDEHLELSTKLTRGEAGDETLEEIERRSREMLEEQRRVDEESKRIREKLK

SEQ ID NO: 285 - protein, 76 aa, synthetic construct
  REGION 1..76: note = Synthetic peptide
SEEEIRDIVEKLLRTHEEVLKEIKKLLDDSERVRRRELDKKDLDRIQKEQRDIQEENKEKAKRFDELVKELKKAAK

SEQ ID NO: 286 - protein, 70 aa, synthetic construct
  REGION 1..70: note = Synthetic peptide
SEEEHRRTMEKVEKEVRDIKRRSEEVKKKVKANTLSEEDLVRLLERLVEDHKRLQDLSQEIIERDEKATK

SEQ ID NO: 287 - protein, 72 aa, synthetic construct
  REGION 1..72: note = Synthetic peptide
DEDELAKEIEDVQRRNKESQEEHDKSVKKLEAAERGEIDEDSLLRVLEEDIKVLEKDIEVLERSIEVIEKAE

SEQ ID NO: 288 - protein, 73 aa, synthetic construct
  REGION 1..73: note = Synthetic peptide
SEKELIRRLLEQQRQHLRLSERLIELSRRLVEVVRKGKDNRDLLRELKKLSEEHKKHSKDDHEKVREIREREK

SEQ ID NO: 289 - protein, 75 aa, synthetic construct
  REGION 1..75: note = Synthetic peptide
DRKDLLKRNIKLLDRHLKILDTILKLLEKLSELLKKSSSEEVVKEYKKILDEIRKLLEESKEIHKESKEILERES

SEQ ID NO: 290 - protein, 75 aa, synthetic construct
  REGION 1..75: note = Synthetic peptide
DEEKLIERSKRLQEESEQLLEKFEQILRELTELLEKPDSEELARKIKKLHDELRKIIKRNQELIREHEEILRKRD

SEQ ID NO: 291 - protein, 82 aa, synthetic construct
  REGION 1..82: note = synthetic peptide
DSDEHLKKLKTFLENLRRHLDRLDKHIKQLRDILSENPEDERVKDVIDLSERSVRIVKTVIKIFEDSVRKKEGSEGSGSEGS

SEQ ID NO: 292 - protein, 85 aa, synthetic construct
  REGION 1..85: note = synthetic peptide
GSDDKELDKLLDTLEKILQTATKIIDDANKLLEKLRRSERKDPKVVETYVELLKRHEKAVKELLEIAKTHAKKVEGSEGSGSEGS

SEQ ID NO: 293 - protein, 87 aa, synthetic construct
  REGION 1..87: note = synthetic peptide
GTKEDILERQRKIIERAQEIHRRQQEILEELERIIRKPGSSEEAMKRMLKLLEESLRLLKELLELSEESAQLLYEQRGSEGSGSEGS

SEQ ID NO: 294 - protein, 87 aa, synthetic construct
  REGION 1..87: note = synthetic peptide
GTEKRLLEEAERAHREQKEIIKKAQELHRRLEEIVRQSGSSEEAKKEAKKILEEIRELSKRSLELLREILYLSQEQKGSEGSGSEGS

SEQ ID NO: 295 - protein, 72 aa, synthetic construct; identical to SEQ ID NO: 263
  REGION 1..72: note = synthetic peptide

SEQ ID NO: 296 - protein, 76 aa, synthetic construct
  REGION 1..76: note = synthetic peptide
DLEDLLRRLRRLVDEQRRLVEELERVSRRLEKAVRDNEDERELARLSREHSDIQDKKDKLAREILEVLKRLLERTE

SEQ ID NO: 297 - protein, 72 aa, synthetic construct; identical to SEQ ID NO: 265
  REGION 1..72: note = synthetic peptide

SEQ ID NO: 298 - protein, 70 aa, synthetic construct; identical to SEQ ID NO: 266
  REGION 1..70: note = synthetic peptide

SEQ ID NO: 299 - protein, 4 aa, synthetic construct
  REGION 1..4: note = synthetic polypeptide
RRRG